(19) United States
(12) Patent Application Publication
(10) Pub. No.: US 2022/0228105 A1
(43) Pub. Date: Jul. 21, 2022

(54) BACTERIUM COMPRISING MYO-INOSITOL TO PROPIONATE PATHWAY

(71) Applicant: WAGENINGEN UNIVERSITEIT, WAGENINGEN (NL)

(72) Inventors: Willem Meindert DE VOS, AMSTERDAM (NL); Thi Phuong Nam BUI, BENNEKOM (NL)

(21) Appl. No.: 17/615,705

(22) PCT Filed: Aug. 14, 2020
(86) PCT No.: PCT/EP2020/072925

§ 371 Date: Dec. 1, 2021

(30) Foreign Application Priority Data
Aug. 14, 2019 (NL) 2023645
Dec. 24, 2019 (NL) 2024569

(51) Int. Cl. C12N 1/20; A61P 3/10; A61K 35/742

(52) U.S. Cl. CPC C12N 1/205; C12R 2001/01; A61K 35/742; A61P 3/10

(57) ABSTRACT

The invention is concerned with a bacterium comprising a inositol to propionic acid pathway gene set that allows said bacterium to convert inositol to propionic acid or a salt or ester thereof. The bacterium can be used as a probiotic or supplement to promote production of propionic acid in the GI tract, thereby preventing and/or treating conditions or diseases that benefit from the production of propionic acid, for example metabolic disease, metabolic syndrome, insulin resistance or insulin resistance-related conditions, particularly dyslipidemia, type 2 diabetes mellitus and insulin-resistance in endocrine diseases.

### TECHNICAL FIELD

The present invention relates to the field of preventing and/or treating metabolic diseases, such as metabolic syndrome and insulin resistance or insulin resistance-related conditions, including dyslipidemia and type 2 diabetes mellitus as well as insulin-resistance in endocrine diseases (e.g., obese subjects with type 1 diabetes mellitus, Cushing's disease or lipodystrophy syndromes).

### BACKGROUND OF THE DISCLOSURE

Insulin resistance is a medical condition wherein cells are resistant to the insulin, leading to a high blood sugar level. Patients with type 2 diabetes mellitus usually have passed through an earlier stage of insulin resistance, although this earlier stage often goes undiagnosed.

Dietary supplementation of myo-inositol has been found to be efficient in lowering post-prandial plasma glucose in several animal models of diabetes or insulin resistance (Ortmeyer et al 1993 Endocrinology 132: 646-651; Ortmeyer et al 1995 Obesity Research 3: 605S-608S; Croze and Soulage 2013 Biochimie 95: 1811-1827).

Subjects with type 2 diabetes mellitus have been shown to display decreased inositol phosphoglycan levels in muscle biopsies as compared to healthy controls, and myo-inositol has been proposed as putative downstream second messenger of the insulin signalling pathway (Kennington et al 1990 New England Journal of Medicine 323: 373-378).

Dietary myo-inositol can be derived from phospholipids or phytic acid (a plant component present in beans, seed and nuts), from which it is liberated by phytases. Moreover, the human body can produce myo-inositol from glucose.

However, although it is known that dietary myo-inositol supplementation can reduce insulin resistance, the mechanism through which this occurs is unknown.

It is an objective of the present disclosure to provide a new or improved strategy for preventing and/or treating metabolic diseases, in particular metabolic syndrome and insulin resistance or insulin resistance-related conditions, including dyslipidemia and type 2 diabetes mellitus as well as insulin-resistance in endocrine diseases (e.g., obese subjects with type 1 diabetes mellitus, Cushing's disease or lipodystrophy syndromes).

### SUMMARY OF THE DISCLOSURE

The present inventors found that myo-inositol can serve as carbon and energy source for a butyrogenic bacterium Anaerostipes rhamnosivorans that was isolated from stool of a healthy infant (Bui et al 2014 International Journal of Systematic and Evolutionary Microbiology 64: 787-793). This bacterium is able to ferment efficiently myo-inositol to propionic acid and a complete inositol fermentation pathway has been found in its closed genome, and which optionally can be introduced in any other bacterium. See FIGS. 2A and 2B.

It is known that dietary myo-inositol supplementation can reduce insulin resistance. However, the mechanism through which this occurs was unknown before the present invention was made.

The present inventors considered that myo-inositol may have an indirect beneficial effect, and that it is the metabolic conversion of myo-inositol that is beneficial to the subject, rather than the myo-inositol itself. Hence, it is of interest to note that the production of butyrate and, notably, propionate by A. rhamnosivorans is certainly beneficial for the subject (see e.g. Hamer et al 2008 Pharmacology & Therapeutics 27: 104-119; Hosseini et al 2011 Nutrition Reviews 69: 245-258).

The present inventors envisage supplementation of a subject with a bacterium according to the present disclosure, or alternatively both myo-inositol and a bacterium according to the present disclosure, to provide for a direct or synergistic effect leading to improved insulin resistance via in situ propionic acid and optionally butyric acid production.

### DETAILED DESCRIPTION OF THE DISCLOSURE

The present disclosure relates to a bacterium comprising a gene set encoding an inositol to propionic acid pathway that allows said bacterium to convert inositol to propionic acid or a salt or ester thereof, for use in preventing and/or treating metabolic syndrome, insulin resistance and/or insulin resistance related condition.

Specifically, the bacterium is capable of converting inositol into propionic acid or a derivative thereof via inositol metabolism and propionic acid synthesis. The inositol may be chiro-inositol or myo-inositol, preferably myo-inositol. In a preferred embodiment, the bacterium additionally is a butyric acid and/or butyrate-producing (butyrogenic) bacterium (i.e. also comprises a butyric acid/butyrate synthesis pathway gene set).

Without wishing to be bound by any theories, it is believed that the bacterium according to the present disclosure (or strains derived therefrom), when administered to a human being or when ingested by a human being in an adequate amount, is able to survive and at least transiently colonize the GI tract of said human being. This colonization enables greater in situ production of propionic acid/propionate (and/or butyric acid/butyrate). Increased in situ production of propionic acid is believed to underlie the beneficial effects as taught herein, e.g. preventing and/or treating conditions or diseases such as metabolic diseases, such as metabolic syndrome and insulin resistance or insulin resistance-related complications, such as dyslipidemia and type 2 diabetes mellitus as well as insulin-resistance in endocrine diseases (e.g., obese subjects with type 1 diabetes mellitus, Cushing's disease or lipodystrophy syndromes).

Bacterium

In a first aspect, the present disclosure relates to a bacterium comprising a gene set encoding an inositol to propionic acid pathway that allows said bacterium to convert inositol to propionic acid or a salt or ester thereof, in particular but not necessarily under anaerobic conditions, such as wherein the bacterium and/or its medium is not in contact with gas comprising more than 1, 2, 3, 4, 5, 10 vol % oxygen. The bacterial strain is preferably, but not necessarily, an isolate, e.g. a human intestinal isolate.

The inositol to propionic acid pathway gene set may comprise one or more (at least two or three, or all) of the genes encoding the proteins: myo-inositol 2-dehydrogenase, inosose dehydratase, epi-inositol hydrolase, 5-deoxy-glucuronate isomerase, 5-keto-2-deoxygluconokinase, 5-keto-2-deoxy-D-gluconate-6 phosphate aldolase, glyceraldehyde-3-phosphate keto-isomerase, glyceraldehyde-3-phosphate dehydrogenase, phosphoglycerate kinase, phosphoglycerate mutase, enolase, pyruvate kinase, pyruvate-flavodoxin oxidoreductase, 3-oxoacid CoA transferase. acyl-CoA dehydrogenase, enoyl-CoA hydratase, and acryloyl-CoA dehydrogenase/Etf. Additionally, the inositol to propionic acid pathway gene set may comprise the gene encoding the protein inosose isomerase and/or another copy of the enzyme myo-inositol 2-dehydrogenase since it allows for conversion of D-chiro-inositol to scyllo-inosose.

Additionally or alternatively, the bacterium according to the present disclosure may comprise a gene encoding lactate dehydrogenase, a gene encoding acetaldehyde dehydrogenase and/or a gene encoding alcohol dehydrogenase.

In a preferred embodiment, at least one (or at least two or three or all) of the proteins: myo-inositol 2-dehydrogenase, inosose dehydratase, epi-inositol hydrolase, 5-deoxy-glucuronate isomerase, 5-keto-2-deoxygluconokinase, 5-keto-2-deoxy-D-gluconate-6 phosphate aldolase, and glyceraldehyde-3-phosphate keto-isomerase, glyceraldehyde-3-phosphate dehydrogenase, phosphoglycerate kinase, phosphoglycerate mutase, enolase, pyruvate kinase, pyruvate-flavodoxin oxidoreductase, 3-oxoacid CoA transferase, acyl-CoA dehydrogenase, enoyl-CoA hydratase, acryloyl-CoA dehydrogenase/Etf and inosose isomerase, may be overproduced (or the genes encoding therefor overexpressed) when the bacterium is grown in the presence of (myo) inositol as compared to when the bacterium is grown in the absence of (myo) inositol.

In the context of the present disclosure, the amount of (myo) inositol used to determine whether genes are upregulated or proteins are overexpressed in a bacterium as compared to when said bacterium is grown in absence of (myo) inositol may be in the range of from about 5 mM to about 100 mM, preferably from about 10 mM to about 50 mM, more preferably from about 15 mM to about 25 mM, and is most preferably about 20 mM.

In an embodiment, the bacterium according to the present disclosure may be an isolated intestinal bacterial strain, or strain derived therefrom, and/or may be an intestinal bacterium isolated from a human being, which naturally comprises an inositol to propionic acid pathway gene set as taught herein and which is capable of converting (myo and/or chiro) inositol to propionic acid or a derivative thereof.

In a further aspect, the bacterium according to the disclosure may be the bacterium Anaerostipes rhamnosivorans 1y2 deposited by Wageningen University on Jun. 26, 2015 at the Centraalbureau voor Schimmelcultures (now named Westerdijk Fungal Biodiversity Institute) located at Uppsalalaan 8, 3584CT Utrecht, the Netherlands, assigned the deposit number CBS 140182. Also encompassed is any bacterial strain derived from the deposited bacterium.

Alternatively, the bacterium according to the present disclosure may be a bacterium which has been transfected with the inositol to propionic acid pathway gene set as taught herein, and which is capable of converting (myo and/or chiro) inositol to propionic acid or a derivative thereof. The skilled person is well-acquainted with methods for transforming bacteria with a desired genetic construct (i.e. pathway gene set).

In an embodiment, the bacterium according to the present disclosure belongs to the phylum Firmicutes, preferably to the class Clostridia (and/or order Clostridiales), more preferably to the family Lachnospiraceae, more preferably to the genus Anaerostipes, even more preferably to the species Anaerostipes rhamnosivorans.

In an embodiment, the bacterium according to the present disclosure is not Anaerostipes hadrus. Alternatively, in case the bacterium according to the present disclosure is Anaerostipes hadrus, the use is for preventing and/or treating insulin resistance or insulin resistance related complication as taught herein. Preferably the Anaerostipes hadrus concerns type strain DSM3319 or 108065. Alternatively, the bacterium according to the present disclosure is Anaerostipes butyraticus, preferably type strain DSM22094.

As set out herein before, the inositol to propionic acid pathway gene set may comprise one or more of the genes encoding the proteins: myo-inositol 2-dehydrogenase, inosose dehydratase, epi-inositol hydrolase, 5-deoxy-glucuronate isomerase, 5-keto-2-deoxygluconokinase, 5-keto-2-deoxy-D-gluconate-6 phosphate aldolase, glyceraldehyde-3-phosphate keto-isomerase, glyceraldehyde-3-phosphate dehydrogenase, phosphoglycerate kinase, phosphoglycerate mutase, enolase, pyruvate kinase, pyruvate-flavodoxin oxidoreductase, 3-oxoacid CoA transferase, acyl-CoA dehydrogenase, enoyl-CoA hydratase, and acryloyl-CoA dehydrogenase/Etf. Additionally, the inositol to propionic acid pathway gene set may comprise the gene encoding the protein inosose isomerase and/or another copy of the enzyme myo-inositol 2-dehydrogenase since each will allow for conversion of D-chiro-inositol to scyllo-inosose.

The gene encoding myo-inositol 2-dehydrogenase may have at least 70, 80, 90, 95, 99, 100% sequence identity with SEQ ID NO:1. Additionally or alternatively the gene encoding myo-inositol 2-dehydrogenase may concern a myo-inositol 2-dehydrogenase that has at least 70, 80, 90, 95, 99, 100% sequence identity with SEQ ID NO:2. The function of the gene myo-inositol 2-dehydrogenase can be seen in the conversion of myo-inositol to scyllo-inosose.

A further or alternative copy of the gene encoding myo-inositol 2-dehydrogenase may have at least 70, 80, 90, 95, 99, 100% sequence identity with SEQ ID NO:3. Additionally or alternatively, the further or alternative copy of the gene encoding myo-inositol 2-dehydrogenase may concern a myo-inositol 2-dehydrogenase that has at least 70, 80, 90, 95, 99, 100% sequence identity with SEQ ID NO:4. The function of this gene can be seen in a conversion from D-chiro-Inositol to 1-keto-D-chiro-Inositol.

The gene encoding inosose dehydratase may have at least 70, 80, 90, 95, 99, 100% sequence identity with SEQ ID NO:5. Additionally or alternatively the gene encoding inosose dehydratase may concern an inosose dehydratase that has at least 70, 80, 90, 95, 99, 100% sequence identity with SEQ ID NO: 6. The function of the gene inosose dehydratase can be seen in the conversion of scyllo-inosose to 3,5/4-trihydroxycyclohexa-1,2-dione.

The gene encoding epi-inositol hydrolase may have at least 70, 80, 90, 95, 99, 100% sequence identity with SEQ ID NO:7. Additionally or alternatively the gene encoding epi-inositol hydrolase may concern an epi-inositol hydrolase that has at least 70, 80, 90, 95, 99, 100% sequence identity with SEQ ID NO:8. The function of the gene epi-inositol hydrolase can be seen in the conversion of 3,5/4-trihydroxycyclohexa-1,2-dione to 5-deoxy-D-glucuronate.

The gene encoding 5-deoxy-glucuronate isomerase may have at least 70, 80, 90, 95, 99, 100% sequence identity with SEQ ID NO:9. Additionally or alternatively the gene encoding 5-deoxy-glucuronate isomerase may concern a 5-deoxy-glucuronate isomerase that has at least 70, 80, 90, 95, 99, 100% sequence identity with SEQ ID NO:10. The function of the gene 5-deoxy-glucuronate isomerase can be seen in the conversion of 5-deoxy-D-glucuronate to 2-deoxy-5-keto-D-gluconate.

The gene encoding 5-keto-2-deoxygluconokinase may have at least 70, 80, 90, 95, 99, 100% sequence identity with SEQ ID NO:11. Additionally or alternatively the gene encoding 5-keto-2-deoxygluconokinase may concern a 5-keto-2-deoxygluconokinase that has at least 70, 80, 90, 95, 99, 100% sequence identity with SEQ ID NO:12. The function of the gene 5-keto-2-deoxygluconokinase can be seen in the conversion of 2-deoxy-5-keto-D-gluconate to 2-deoxy-5-keto-D-gluconate-6P.

The gene encoding 5-keto-2-deoxy-D-gluconate-6 phosphate aldolase may have at least 70, 80, 90, 95, 99, 100% sequence identity with SEQ ID NO:13. Additionally or alternatively the gene encoding 5-keto-2-deoxy-D-gluconate-6 phosphate aldolase may concern a 5-keto-2-deoxy-D-gluconate-6 phosphate aldolase that has at least 70, 80, 90, 95, 99, 100% sequence identity with SEQ ID NO:14. The function of the gene 5-keto-2-deoxy-D-gluconate-6 phosphate aldolase can be seen in the conversion of 2-deoxy-5-keto-D-gluconate-6P to 3-oxopropionate and glycerone phosphate.

The gene encoding glyceraldehyde-3-phosphate keto-isomerase may have at least 70, 80, 90, 95, 99, 100% sequence identity with SEQ ID NO:15. Additionally or alternatively the gene encoding glyceraldehyde-3-phosphate keto-isomerase may concern a glyceraldehyde-3-phosphate keto-isomerase that has at least 70, 80, 90, 95, 99, 100% sequence identity with SEQ ID NO:16. The function of the gene glyceraldehyde-3-phosphate keto-isomerase can be seen in the conversion of glycerone phosphate to glyceraldehyde-3P.

The gene encoding glyceraldehyde-3-phosphate dehydrogenase may have at least 70, 80, 90, 95, 99, 100% sequence identity with SEQ ID NO:17. Additionally or alternatively the gene encoding glyceraldehyde-3-phosphate dehydrogenase may concern a glyceraldehyde-3-phosphate dehydrogenase that has at least 70, 80, 90, 95, 99, 100% sequence identity with SEQ ID NO:18. The function of the gene glyceraldehyde-3-phosphate dehydrogenase can be seen in the conversion of glyceraldehyde-3P to 1,3-biphosphoglycerate.

The gene encoding phosphoglycerate kinase may have at least 70, 80, 90, 95, 99, 100% sequence identity with SEQ ID NO:19. Additionally or alternatively the gene encoding phosphoglycerate kinase may concern a phosphoglycerate kinase that has at least 70, 80, 90, 95, 99, 100% sequence identity with SEQ ID NO:20. The function of the gene phosphoglycerate kinase can be seen in the conversion of 1,3-biphosphoglycerate to 3-phosphoglycerate.

The gene encoding phosphoglycerate mutase may have at least 70, 80, 90, 95, 99, 100% sequence identity with SEQ ID NO:21. Additionally or alternatively the gene encoding phosphoglycerate mutase may concern a phosphoglycerate mutase that has at least 70, 80, 90, 95, 99, 100% sequence identity with SEQ ID NO:22. The function of the gene phosphoglycerate mutase can be seen in the conversion of 3-phosphoglycerate to 2-phosphoenol pyruvate.

The gene encoding enolase may have at least 70, 80, 90, 95, 99, 100% sequence identity with SEQ ID NO:23. Additionally or alternatively the gene encoding enolase may concern a enolase that has at least 70, 80, 90, 95, 99, 100% sequence identity with SEQ ID NO:24. The function of the gene enolase can be seen in the conversion of 3-phosphoglycerate to 2-phosphoenolpyruvate.

The gene encoding pyruvate kinase may have at least 70, 80, 90, 95, 99, 100% sequence identity with SEQ ID NO:47. Additionally or alternatively the gene encoding pyruvate kinase may concern a pyruvate kinase that has at least 70, 80, 90, 95, 99, 100% sequence identity with SEQ ID NO:48. The function of the gene pyruvate kinase can be seen in the conversion of phosphoenolpyruvate to pyruvate.

The gene encoding pyruvate-flavodoxin oxidoreductase may have at least 70, 80, 90, 95, 99, 100% sequence identity with SEQ ID NO:25. Additionally or alternatively the gene encoding pyruvate-flavodoxin oxidoreductase may concern a pyruvate-flavodoxin oxidoreductase that has at least 70, 80, 90, 95, 99, 100% sequence identity with SEQ ID NO:26. The function of the gene pyruvate-flavodoxin oxidoreductase can be seen in the conversion of pyruvate to acetyl-CoA and CO₂/formic acid.

The gene encoding 3-oxoacid CoA transferase may have at least 70, 80, 90, 95, 99, 100% sequence identity with SEQ ID NO:27. Additionally or alternatively the gene encoding 3-oxoacid CoA transferase may concern a 3-oxoacid CoA transferase that has at least 70, 80, 90, 95, 99, 100% sequence identity with SEQ ID NO:28. The function of the gene 3-oxoacid CoA transferase can be seen in the conversion of 3-oxopropionate to 3-oxopropionyl-CoA and/or in the conversion of propionyl-CoA to propionate.

The gene encoding acyl-CoA dehydrogenase may have at least 70, 80, 90, 95, 99, 100% sequence identity with SEQ ID NO:29. Additionally or alternatively the gene encoding acyl-CoA dehydrogenase may concern a acyl-CoA dehydrogenase that has at least 70, 80, 90, 95, 99, 100% sequence identity with SEQ ID NO:30. The function of the gene acyl-CoA dehydrogenase can be seen in the conversion of 3-oxopropionyl-CoA to 3-hydroxy propionyl-CoA.

The gene encoding enoyl-CoA hydratase may have at least 70, 80, 90, 95, 99, 100% sequence identity with SEQ ID NO:31. Additionally or alternatively the gene encoding enoyl-CoA hydratase may concern a enoyl-CoA hydratase that has at least 70, 80, 90, 95, 99, 100% sequence identity with SEQ ID NO:32. The function of the gene enoyl-CoA hydratase can be seen in the conversion of 3-hydroxy propionyl-CoA to acryloyl-CoA.

The gene encoding acryloyl-CoA dehydrogenase/Etf may have at least 70, 80, 90, 95, 99, 100% sequence identity with SEQ ID NO:33. A further (second) or alternative copy of the gene encoding acryloyl-CoA dehydrogenase/Etf may have at least 70, 80, 90, 95, 99, 100% sequence identity with SEQ ID NO:35. A further (third) or alternative copy of the gene encoding acryloyl-CoA dehydrogenase/Etf may have at least 70, 80, 90, 95, 99, 100% sequence identity with SEQ ID NO:37. Additionally or alternatively the gene encoding acryloyl-CoA dehydrogenase/Etf may concern a acryloyl-CoA dehydrogenase/Etf that has at least 70, 80, 90, 95, 99, 100% sequence identity with SEQ ID NO:34. A further (second) or alternative copy of the gene encoding acryloyl-CoA dehydrogenase/Etf may concern a acryloyl-CoA dehydrogenase/Etf that has at least 70, 80, 90, 95, 99, 100% sequence identity with SEQ ID NO:36. A further (third) or alternative copy of the gene encoding acryloyl-CoA dehydrogenase/Etf may concern a acryloyl-CoA dehydrogenase/Etf that has at least 70, 80, 90, 95, 99, 100% sequence identity with SEQ ID NO:38. Acryloyl-CoA dehydrogenase/Eft can be a complex in which AR1Y2_1051 and AR1Y2_1052 are the alpha and beta subunit of a complex that function as the electron acceptor of the acryolyl-CoA dehydrogenase (AR1Y2_1050). The function of the gene acryloyl-CoA dehydrogenase/Etf can be seen in the conversion of acryloyl-CoA to propionyl-CoA.

The gene encoding inosose isomerase may have at least 70, 80, 90, 95, 99, 100% sequence identity with SEQ ID NO:39. Additionally or alternatively the gene encoding inosose isomerase may concern a inosose isomerase that has at least 70, 80, 90, 95, 99, 100% sequence identity with SEQ ID NO:40. The function of this gene can be seen in a conversion from 1-keto-D-chiro-Inositol to scyllo-inosose.

The gene encoding lactate dehydrogenase may have at least 70, 80, 90, 95, 99, 100% sequence identity with SEQ ID NO:41. Additionally or alternatively the gene lactate dehydrogenase may concern a lactate dehydrogenase that has at least 70, 80, 90, 95, 99, 100% sequence identity with SEQ ID NO:42. The function of the gene lactate dehydrogenase can be seen in the conversion of pyruvate to lactate.

The gene encoding acetaldehyde dehydrogenase may have at least 70, 80, 90, 95, 99, 100% sequence identity with SEQ ID NO:43. Additionally or alternatively the gene acetaldehyde dehydrogenase may concern an acetaldehyde dehydrogenase that has at least 70, 80, 90, 95, 99, 100% sequence identity with SEQ ID NO:44. The function of the gene acetaldehyde dehydrogenase can be seen in the conversion of acetyl-CoA to acetaldehyde.

The gene encoding alcohol dehydrogenase may have at least 70, 80, 90, 95, 99, 100% sequence identity with SEQ ID NO:45. Additionally or alternatively the gene alcohol dehydrogenase may concern an alcohol dehydrogenase that has at least 70, 80, 90, 95, 99, 100% sequence identity with SEQ ID NO:46. The function of the gene alcohol dehydrogenase can be seen in the conversion of acetaldehyde to ethanol.

In certain embodiments, the bacterium according to the present disclosure does not comprise a gene encoding malonate-semialdehyde dehydrogenase (involved in the conversion of malonic semi-aldehyde to acetyl-CoA).

Compositions

The present disclosure further provides a composition comprising a bacterium according to the present disclosure and optionally a physiologically acceptable carrier. The physiologically acceptable carrier may be any carrier that is suitable for keeping the bacterium as taught herein viable until consumption by a subject (e.g. human or animal). For instance, non-limiting examples of acceptable carriers that are suitable for this purpose include any of well-known physiological or pharmaceutical carriers, buffers, and excipients. It will be appreciated that the choice for a suitable physiological or pharmaceutical carrier will depend upon the intended mode of administration of the composition as taught herein (e.g. oral) and the intended form of the composition (e.g. beverage, yogurt, powder, capsules, and the like). The skilled person knows how to select a physiological or pharmaceutical carrier, which is suitable for the compositions as taught herein.

In an embodiment, the composition as taught herein may be in the form of a food composition, feed composition, feed supplement composition, food supplement composition or pharmaceutical composition. The composition is preferably suitable for consumption by a human being.

In an embodiment, the composition is a food or food supplement composition. The food or food supplement composition may be selected from the group consisting of a liquid, liquid beverage (including dairy beverage and fermented beverage), yogurt, cheese, gel, gelatine, gelatine capsule, powder, paste, pressed tablet, and gel cap. In a suitable embodiment, the composition is a liquid, preferably a liquid beverage (e.g. dairy beverage). The food or food supplement composition may be a dairy product, preferably a fermented dairy product, preferably a yogurt or a yogurt drink.

In an embodiment, the composition as taught herein may be a probiotic composition. Such probiotic composition may comprise the (isolated) bacterium as taught herein, or a strain derived therefrom. In an embodiment, the composition as taught herein further comprises one or more additional beneficial isolated intestinal bacterial strain.

In one embodiment, the one or more additional beneficial isolated intestinal bacterial strain may be any lactic acid bacterial strain selected from the genera Lactobacillus and/or Bifidobacterium and/or any butyrate-producing bacteria which produces butyrate via the acetyl-CoA pathway.

In an embodiment, the composition may be a symbiotic composition. It may be advantageous to add one or more prebiotic ingredients to the composition as taught herein, for example, to supplement the effects (e.g. production of propionic acid/propionate and/or butyric acid/butyrate or a derivative thereof) of the bacterium as taught herein.

In an embodiment, the one or more prebiotic ingredients may be any prebiotic ingredients, which are suitable to enhance the activity and/or stimulate the growth of the bacterium, or a strain derived therefrom, as taught herein. Non-limiting examples of suitable prebiotic ingredients include fibres such as inulin, pectin, and resistant starch, as well as cellobiose, maltose, mannose, salicine, trehalose, amygdalin, arabinose, melibiose, sorbitol, rhamnose and/or xylose.

In an embodiment, the composition as taught herein comprises an (myo)inositol-rich source and/or (myo) inositol. For instance, it may be advantageous to add a (myo)inositol rich source and/or (myo) inositol to the composition as taught herein to further promote the production of propionic acid/propionate and/or butyric acid/butyrate or a derivative thereof in the GI tract of a mammal (e.g. human being).

In an embodiment, the composition as taught herein may comprise one or more ingredients which are suitable for promoting survival and/or viability of the bacterium or strain derived therefrom as taught herein during storage and/or during exposure to bile and/or during passage through the GI tract of a mammal (e.g. a human being). Non-limiting examples of such ingredients include an enteric coating, and controlled release agents allowing passage through the stomach. The skilled person knows how to select suitable ingredients for maintaining a bacterium as taught herein viable and functional i.e. able to carry out intended function(s).

In one embodiment, the compositions as taught herein may further comprise one or more ingredients, which further enhance the nutritional value and/or the therapeutic value the compositions as taught herein. For instance, it may be advantageous to add one or more ingredients (e.g. nutritional ingredients, veterinary or medicinal agents etc.) selected from proteins, amino acids, enzymes, mineral salts, vitamins (e.g. thiamine HCl, riboflavin, pyridoxine HCl, niacin, inositol, choline chloride, calcium pantothenate, biotin, folic acid, ascorbic acid, vitamin B12, p-aminobenzoic acid, vitamin A acetate, vitamin K, vitamin D, vitamin E, and the like), sugars and complex carbohydrates (e.g. water-soluble and water-insoluble monosaccharides, disaccharides, and polysaccharides), medicinal compounds (e.g. antibiotics), antioxidants, trace element ingredients (e.g. compounds of cobalt, copper, manganese, iron, zinc, tin, nickel, chromium, molybdenum, iodine, chlorine, silicon, vanadium, selenium, calcium, magnesium, sodium and potassium and the like). The skilled person is familiar with methods and ingredients that are suitable to enhance the nutritional and/or therapeutic/medicinal value of the compositions as taught herein.

The bacterium as taught herein may be incorporated into the composition in lyophilized form, microencapsulated form (reviewed by, for example, Solanki et al. BioMed Res. Int. 2013, Article ID 620719), or any other form preserving the activity and/or viability of the bacterial strain.

The composition as taught herein may be a pharmaceutical composition. The pharmaceutical composition may be for use as a supplement. A pharmaceutical composition will usually comprise a pharmaceutical carrier, in addition to the bacterium taught herein. The carrier is preferably an inert carrier. The preferred form depends on the intended mode of administration and (therapeutic) application. A pharmaceutical carrier can be any compatible, nontoxic substance suitable to deliver the bacterium taught herein to the GI tract of a subject. For example, sterile water, or inert solids may be used as a carrier, usually complemented with a pharmaceutically acceptable adjuvant, buffering agent, dispersing agent, and the like. A pharmaceutical composition as taught herein may be in liquid form, e.g. a stabilized suspension of bacteria of the bacterium taught herein, or in solid form, e.g., a powder of lyophilized bacteria taught herein. In case the bacterium taught herein is lyophilized, a cryoprotectant such as lactose, trehalose or glycogen can be employed. E.g., for oral administration, the bacterium taught herein can be administered in solid dosage forms, such as capsules, tablets, and powders, comprising lyophilized bacteria, or in liquid dosage forms, such as elixirs, syrups, and suspensions. The bacterium taught herein, e.g., in lyophilized form, can be encapsulated in capsules such as gelatin capsules, together with inactive ingredients and powdered carriers, such as e.g. glucose, lactose, sucrose, mannitol, starch, cellulose or cellulose derivatives, magnesium stearate, stearic acid, sodium saccharin, talcum, magnesium carbonate and the like.

In an embodiment, the bacterium as taught herein may be comprised in the composition as taught herein in an amount ranging from about 10⁶ to about 10¹⁵ colony forming units (CFU). For instance, the intestinal bacteria may be comprised in the composition in an amount of about 10⁷ CFU to about 10¹⁴ CFU, preferably about 10⁸ CFU to about 10¹³ CFU, preferably about 10⁹ CFU to about 10¹² CFU, more preferably about 10¹⁰ CFU to about 10¹² CFU.

Methods and Uses of the Disclosure

The present disclosure is concerned with a bacterium as taught herein or a composition as taught herein for use as a medicament, for use as a food or food supplement, or for use as a probiotic and/or symbiotic.

In a preferred embodiment, the bacterium for use according to the present disclosure may be administered separately, sequentially or simultaneously with (myo) inositol, or with a (myo) inositol source (or a phospholipid or phytic acid source) which may be present in the same composition as the bacterium, or in a separate composition, for example comprising in the range of from about 5 mM to about 100 mM, preferably from about 10 mM to about 50 mM, more preferably from about 15 mM to about 25 mM, and is most preferably about 20 mM (myo)inositol, or for example comprising from about 1 mg to about 1000 mg, preferably from about 10 mg to about 500 mg, more preferably from about 15 mg to about 50 mg. Alternatively, the amount of inositol and/or administration frequency is chosen such to that between 0.1 and 100 grams is consumed per day, or between 0.5 and 10, or between 0.5 and 10, or between 0.1 and 5, all in grams per day.

The present disclosure particularly pertains to the bacterium as taught herein or a composition as taught herein for use in maintaining, restoring and/or improving GI health in general, and/or for preventing and/or treating conditions or diseases such as obesity, metabolic diseases, such as metabolic syndrome and insulin resistance or insulin resistance-related complications, such as dyslipidemia and type 2 diabetes mellitus as well as insulin-resistance in endocrine diseases (e.g., obese subjects with type 1 diabetes mellitus, Cushing's disease or lipodystrophy syndromes).

The present disclosure is also directed to a method for maintaining, restoring and/or improving GI health in general, and/or for preventing and/or treating conditions or diseases such as obesity, metabolic diseases, such as metabolic syndrome and insulin resistance or insulin resistance-related complications, such as dyslipidemia and type 2 diabetes mellitus as well as insulin-resistance in endocrine diseases (e.g., obese subjects with type 1 diabetes mellitus, Cushing's disease or lipodystrophy syndromes) in a subject in need thereof, said method comprising the step of increasing the level of the bacterium as taught herein in said subject.

The level of the bacterium as taught herein in said subject may be increased by administering an effective amount of said bacterium to said subject, and/or by administering an effective amount of a compound capable of increasing the level of said bacterium in (the GI tract of) said subject.

In an embodiment, the bacterium as taught herein may be administered concomitant with (myo)inositol or a (myo)inositol source (or a phospholipid or phytic acid source), such as fruits, beans, grains, and nuts. The skilled person can, without undue burden, readily identify (myo)inositol-rich sources.

In a preferred embodiment, the level of the bacterium as taught herein in the GI tract of a subject may be increased by administering an effective amount of the bacterium as taught herein, but preferably Anaerostipes rhamnosivorans 1y2 and/or a composition as taught herein comprising Anaerostipes rhamnosivorans 1y2, to said subject.

In an embodiment, the subject may be selected from the group consisting of human beings, non-human primates, mice, rats, dogs, cows, and pigs. In a preferred embodiment, the subject is a human.

The disclosure also relates to a method for producing propionic acid, said method comprising the step of contacting the bacterium as taught herein with a suitable energy source, e.g. (myo)inositol and/or glucose/acetate, under conditions which allow the bacterium as taught herein to convert the energy source to propionic acid.

The methods taught herein may be in vitro methods.

Clauses

1. Bacterium comprising a gene set that allows said bacterium to convert inositol to propionic acid or a salt or ester thereof, for use in preventing and/or treating metabolic syndrome, insulin resistance or insulin resistance-related condition.

2. Bacterium for use according to clause 1, wherein the gene set comprises genes encoding one of more of the proteins: myo-inositol 2-dehydrogenase, inosose dehydratase, epi-inositol hydrolase, 5-deoxy-glucuronate isomerase, 5-keto-2-deoxygluconokinase, 5-keto-2-deoxy-D-gluconate-6 phosphate aldolase, glyceraldehyde-3-phosphate ketol-isomerase, and inosose isomerase.

3. Bacterium for use according to any one of the previous clauses, wherein at least one of the genes encoding the proteins: myo-inositol 2-dehydrogenase, inosose dehydratase, epi-inositol hydrolase, 5-deoxy-glucuronate isomerase, 5-keto-2-deoxygluconokinase, 5-keto-2-deoxy-D-gluconate-6 phosphate aldolase, glyceraldehyde-3-phosphate ketol-isomerase, and inosose isomerase, is overexpressed when the bacterium is grown in the presence of inositol as compared to when the bacterium is grown in the absence of inositol.

4. Bacterium for use according to any one of the previous clauses, wherein the use is in preventing and/or treating insulin resistance or insulin resistance-related condition.

5. Bacterium for use according to any one of the previous clauses, wherein the insulin resistance-related condition is chosen from dyslipidemia, type 2 diabetes mellitus and insulin-resistance in endocrine disease.

6. Bacterium for use according to any one of the previous clauses, wherein the bacterium is a human intestinal isolate bacterium.

7. Bacterium for use according to any one of the previous clauses, wherein the bacterium is deposited as CBS 140182.

8. Bacterium for use according to any one of the previous clauses, wherein the bacterium is comprised in a composition further comprising a physiologically acceptable carrier.

9. Bacterium for use according to clause 8, wherein the composition is a food composition, preferably a dairy product, more preferably a fermented dairy product, preferably a yogurt or a yogurt drink.

10. Bacterium for use according to clause 8, wherein the composition is a pharmaceutical composition, preferably in solid dosage form, preferably a capsule, a tablet, or a powder.

11. Bacterium for use according to any one of clauses 8-10, wherein in the composition the bacterium is present in lyophilized or microencapsulated form.

12. Bacterium for use according to any one of the previous clauses, wherein the bacterium is present in an amount within the range of between 10⁶ to 10¹⁵ colony forming units (CFU)

13. Bacterium for use according to any one of the previous clauses, wherein the bacterium is administered separately, sequentially or simultaneously with inositol.

14. Bacterium for use according to any one of the previous clauses for use as a probiotic.

### General Definitions

The term ‘probiotics’ or ‘probiotic products’ as used herein refers to microorganisms such as intestinal bacteria, which—when administered or ingested in effective amounts—confer health benefits to the host (e.g. humans or mammals). Preferably, probiotics should be alive or viable when administered to a subject so as to allow the probiotics to colonize the large intestine of the host. However, under certain conditions, probiotics may also be dead when administered provided that substances produced by the probiotics still exert probiotic, beneficial effects on the host. Most probiotics or probiotic products are composed of lactic acid bacteria such as Lactobacilli or Bifidobacteria. The skilled person is well-acquainted with the field of probiotics and knows how to select lactic acid bacteria endowed with probiotic activity.

The term ‘prebiotics’ or ‘prebiotic products’ as used herein generally refers to compounds that promote the growth and/or activity of GI microorganisms that contribute to the well-being of their host. Prebiotics or prebiotic products consist mainly of fermentable fibres or non-digestible carbohydrates. The fermentation of these fibres by probiotics promotes the production of beneficial end products, such as SCFAs, particularly butyrate. The skilled person is well-acquainted with the field of prebiotics and knows how to select ingredients endowed with prebiotic activity.

The term ‘symbiotics’ or ‘symbiotic products’ as used herein generally refers to compositions and/or nutritional supplements combining probiotics and one or more compounds that promote the growth and/or activity of GI microorganisms, such as prebiotics, into one product. The symbiotic beneficially affects the host by improving the survival and colonization of the probiotic in the GI tract, by selectively stimulating the growth and/or by activating the metabolism of the probiotic, thus improving host welfare. The skilled person is well-acquainted with symbiotics and knows how to select ingredients that may be combined into a symbiotic.

The term ‘propionic acid’ as used herein refers is a carboxylic acid with chemical formula CH₃CH₂CO₂H. The term may include derivatives thereof, i.e. compounds derived from propionic acid and in particular salts and esters of propionic acid which are known as propionates or propanoates. Non-limiting examples of propionate salts are ammonium propionate, calcium propionate, magnesium propionate, potassium propionate and sodium propionate. Non-limiting example of propionate ester is ethyl propionate.

The term ‘butyric acid’ (also known under the systematic name butanoic acid) as used herein refers to a carboxylic acid with the structural formula CH₃CH₂CH₂COOH. The term may include derivatives thereof, i.e. compounds derived from butyric acid and includes salts and esters of butyric acid, which are known as butyrate or butanoate. Non-limiting examples of butyrate salts include sodium butyrate, calcium butyrate, magnesium butyrate, manganese butyrate, cobalt butyrate, barium butyrate, lithium butyrate, zinc butyrate, potassium butyrate, ferrous butyrate and the like. Non-limiting examples of butyrate esters (i.e. esters of butyric acid) include cellulose acetate butyrate, methyl butyrate, ethyl butyrate, butyl butyrate, pentyl butyrate, and the like.

The terms ‘butyrate-producing bacterium’ or ‘butyric acid-producing bacterium’ or ‘butyrogenic bacterium’ are used interchangeably herein and refer to a bacterium which is capable of producing butyric acid and/or butyrate and/or one or more derivatives thereof. A prominent pathway by which butyric acid and/or butyrate and derivative thereof may be produced in situ in the mammalian gut (or in vitro in culture), i.e. a butyric acid/butyrate synthesis pathway gene set, is the so-called ‘acetyl-CoA pathway’. The acetyl-CoA pathway has been well-documented and is known to be particularly prevalent in intestinal bacteria belonging, for instance, to the genus Lachnospiraceae and Ruminococcaceae (which together may form up to 20% of total gut microbiota). According to the acetyl-CoA pathway, butyric acid and/or butyrate and/or derivatives thereof may be formed by a single bacterial species via carbohydrate fermentation and/or by a group of microorganisms where metabolites from other organisms act as a substrate for butyrogenic bacteria. The conventional acetyl-CoA pathway involves a cascade of enzymes, including (among many others) two key enzymes referred to as butyryl-CoA transferase (But) and butyrate kinase (Buk). The skilled person is well-acquainted with the ‘acetyl-CoA pathway’ including genes coding for enzymes and other elements underlying the functioning of said pathway as well as intestinal bacterial species that have this pathway.

The term Inositol to propionic acid (or propionate) pathway gene set′ as used herein refers to a set of genes that encode proteins involved in the conversion of (myo) inositol into propionic acid (propionate) or a derivative thereof. In an embodiment of the present disclosure, the pathway gene set comprises genes encoding the proteins: myo-inositol 2-dehydrogenase, inosose dehydratase, epi-inositol hydrolase, 5-deoxy-glucuronate isomerase, 5-keto-2-deoxygluconokinase, 5-keto-2-deoxy-D-gluconate-6 phosphate aldolase, glyceraldehyde-3-phosphate keto-isomerase, glyceraldehyde-3-phosphate dehydrogenase, phosphoglycerate kinase, phosphoglycerate mutase, enolase, pyruvate kinase, pyruvate-flavodoxin oxidoreductase, 3-oxoacid CoA transferase. acyl-CoA dehydrogenase, enoyl-CoA hydratase, acryloyl-CoA dehydrogenase/Etf and optionally inosose isomerase and/or a second copy of myo-inositol 2-dehydrogenase.

The term ‘beneficial intestinal bacteria species’ as used herein refers to a bacterium species that inhabits (i.e. is innate) the mammalian (e.g. human) intestine and exerts beneficial effect(s) (e.g. protection against pathogenic bacteria species, production of butyric acid and/or butyrate and derivatives, etc.) on the GI, metabolic and other health of a mammal in which it resides. Non-limiting examples of beneficial intestinal bacterial species include lactic acid bacteria from the genera Lactobacillus and Bifidobacterium. Other non-limiting examples of beneficial intestinal bacterial species include butyrate-producing bacterial species, which use the acetyl-CoA to produce butyric acid and/or butyrate and derivative thereof, such as the bacterial strains disclosed in US2014/0242654, WO 2014/150094 or WO2013032328 A1. Pathogenic bacterial species may refer to a bacterium that inhabits (i.e. is innate) the mammalian (e.g. human) intestine and exerts deleterious effect(s) (e.g. infection) on the GI health of a mammal in which it resides. A notorious non-limiting example of a pathogenic bacterial species is the toxin-producing Clostridium difficile.

The term ‘effective amount’ as used herein refers to an amount necessary to achieve an effect as taught herein. For instance, an effective amount of the bacterium as taught herein is an amount which is effectively useful for maintaining, restoring, and/or improving GI heath in a human being, for converting (myo)inositol into propionic acid/propionate or a derivative thereof and/or butyric acid/butyrate or a derivative thereof and/or for preventing and/or treating conditions or diseases described herein, in a subject, preferably a human being. These conditions or diseases include, without limitation, obesity, metabolic diseases, such as metabolic syndrome and insulin resistance or insulin resistance-related complications, such as dyslipidemia and type 2 diabetes mellitus as well as insulin-resistance in endocrine diseases (e.g., obese subjects with type 1 diabetes mellitus, Cushing's disease or lipodystrophy syndromes). The effective amount can be readily determined without undue experimentation by a person of ordinary skill in the art.

The term ‘a strain that derives therefrom’ as used herein relates to strains obtained by using the deposited strain as taught herein as starting material. The strain that derives therefrom may be a mutant strain, which may be derived from a strain of the invention by means of, for instance, genetic engineering, radiation, UV light, chemical treatment. Alternatively, such derivative or mutant strain may be a strain derived from the deposited strain as taught herein that has been subjected to growth adaptation to particular conditions resulting in an additional benefit to the derivative strain, such as more rapid growth, better survival in the gut, enhanced inositol to propionate conversion, using methods that are well-known to the skilled person. It is preferred that the derivative or mutant is functionally equivalent to the deposited strain as taught herein. A preferred derivative or mutant as taught herein has substantially the same activity or function as the deposited strain as taught herein, i.e. has the ability to convert (myo)inositol to propionic acid/propionate and derivatives. The derivative or mutant advantageously provides substantially the same benefits to a mammal (e.g. humans or other mammals) administered with said derivative or mutant as would be the case upon administration of the deposited strain. The derivative or mutant strain may also be a spontaneous derivative or mutant strain having the same characteristics as described herein for the deposited strain.

The term ‘suitable for consumption’ or ‘nutritionally acceptable’ refers to ingredients or substances, which are generally regarded as safe for human (as well as other mammals) consumption.

The term ‘metabolic syndrome’ is well-known by the skilled person. Within the present disclosure, the term encompasses all conditions diagnosed as “metabolic syndrome” by an (authorized) medical practitioner. For example, metabolic syndrome may be diagnosed if a patient has at least two, or at least three of the following traits:
Large waist—A waistline that measures at least 35 inches (89 centimeters) for women and 40 inches (102 centimeters) for men;
High triglyceride level—150 milligrams per deciliter (mg/dL), or 1.7 millimoles per liter (mmol/L), or higher of this type of fat found in blood;
Reduced “good” or HDL cholesterol—Less than 40 mg/dL (1.04 mmol/L) in men or less than 50 mg/dL (1.3 mmol/L) in women of high-density lipoprotein (HDL) cholesterol;
Increased blood pressure—130/85 millimeters of mercury (mm Hg) or higher;
Elevated fasting blood sugar—100 mg/dL (5.6 mmol/L) or higher.

The term ‘insulin resistance’ is well known by the skilled person. Within the present disclosure, the term encompasses all conditions diagnosed as “insulin resistance” by an (authorized) medical practitioner. For example, insulin resistance may be diagnosed by the gold standard for determining and quantifying insulin resistance which is the “hyperinsulinemic euglycemic clamp” (DeFronzo R A, Tobin J D, Andres R 1979). The American Journal of Physiology. 237 (3): E214-23). This method measures the amount of glucose necessary to compensate for an increased insulin level without causing hypoglycemia. The procedure may take about two hours and typically involves the following steps. Through a peripheral vein, insulin is infused at 10-120 mU per m² per minute. In order to compensate for the insulin infusion, glucose 20% is infused to maintain blood sugar levels between 5 and 5.5 mmol/L. The rate of glucose infusion is determined by checking the blood sugar levels every five to ten minutes (Muniyappa R, Lee S, Chen H, Quon M J (January 2008). American Journal of Physiology. Endocrinology and Metabolism. 294 (1): E15-26). The rate of glucose infusion during the last thirty minutes of the test determines insulin sensitivity. If high levels (7.5 mg/min or higher) are required, the patient is insulin-sensitive. Low levels (4.0 mg/min or lower) indicate insulin resistance. Levels between 4.0 and 7.5 mg/min are not definitive, and suggest “impaired glucose tolerance,” an early sign of insulin resistance. Alternatively, the homeostatic model assessment (HOMA) is a method used to determine and quantify insulin resistance, as it correlates reasonably with the golden standard. See e.g. Matthews D R, Hosker J P, Rudenski A S, Naylor B A, Treacher D F, Turner R C (1985). Diabetologia. 28 (7): 412-9. doi:10.1007/BF00280883. PMID 3899825; and/or A. S. Rudenski; D. R. Matthews; J. C. Levy; R. C. Turner (1991) Metabolism. 40 (9): 908-917. A HOMA(-IR) score that deviates from a reference range can indicate insulin resistance. Alternatively, a fasting serum insulin level greater than 25 mU/L or 174 pmol/L can be considered as indicating insulin resistance. The term “insulin resistance” as used herein refers to peripheral insulin resistance and/or hepatic insulin resistance. The term ‘insulin resistance-related conditions’ (or complications) may refer to dyslipidemia. Alternatively or additionally, the term may refer to type 2 diabetes mellitus. Further, the term ‘insulin resistance-related conditions’ (or complications) may refer to insulin-resistance in endocrine disease (e.g., obese subjects with type 1 diabetes mellitus, Cushing's disease or lipodystrophy syndromes).

The term ‘about’, as used herein indicates a range of normal tolerance in the art, for example within 2 standard deviations of the mean. The term ‘about’ can be understood as encompassing values that deviate at most 10%, 9%, 8%, 7%, 6%, 5%, 4%, 3%, 2%, 1%, 0.5%, 0.1%, 0.05%, or 0.01% of the indicated value.

The terms ‘comprising’ or ‘to comprise’ and their conjugations, as used herein, refer to a situation wherein said terms are used in their non-limiting sense to mean that items following the word are included, but items not specifically mentioned are not excluded. It also encompasses the more limiting verb ‘to consist essentially of’ and ‘to consist of’.

Reference to an element by the indefinite article ‘a’ or ‘an’ does not exclude the possibility that more than one of the elements is present, unless the context clearly requires that there be one and only one of the elements. The indefinite article ‘a’ or ‘an’ thus usually means ‘at least one’.

The terms ‘to increase’ and ‘increased level’ and the terms ‘to decrease’ and ‘decreased level’ refer to the ability to significantly increase or significantly decrease or to a significantly increased level or significantly decreased level. Generally, a level is increased or decreased when it is at least 5%, such as 10%, 15%, 20%, 25%, 30%, 35%, 40%, 45%, 50% higher or lower, respectively, than the corresponding level in a control or reference. Alternatively, a level in a sample may be increased or decreased when it is statistically significantly increased or decreased compared to a level in a control or reference.

### BRIEF DESCRIPTION OF THE FIGURES

FIG. 1. A bacterium according to the present disclosure is able to ferment efficiently myo-inositol to propionate. The experiment was performed twice.

FIGS. 2A/2B. the identified inositol to propionate pathway.

FIG. 3A/3B. shows the capabilities of metabolizing myo-inositol by different Anaerostipes strains

FIG. 4. the presence of inositol pathway genes in NCO genomes of Anaerostipes isolates

Sequence listing
Myo-inositol 2-dehydrogenase 1 - AR1Y2_0317
Nucleotide sequence
(SEQ ID NO: 1)
atggtaaatgtaggtattatcggagcaggaagaatcggaagagtacatgtagaaagcatctgcacacaggtaggaaacgca
aaggttaagactcttgctgatccttttatgtcagaggacacagcagtatgggcaaaagaaatgggtgttgaaaataccaccaaa
gattacaaagacatcttaaatgatcctcagatcgacgcagtgctgatctgctcatccacagatacacattcaccgatctccgtag
aggctattgaagcaggaaaacatgtattctgcgaaaagccaatcgaccatgacgtacagaagattcaggaagtagtcgacg
ctttaaagaaaagcgacgtaaaataccaggtaggattcaaccgccgttttgaccataactttgaagccatccgggatgccgtgg
ctgctggaaagattggtgagacacagatcatcaaagttacatccagagatcctgaaccgccaagtgctgattatgtaaaagttt
ccggaggaatgttcctggacatgacaatccatgattttgacatggtccgtttccttgcaggatgtgacgctgaagaagtgtatgta
gaagctgcagtccttgtagatcctgccatcggtgaagccggagatgtggacactgctgttatcacattaaaaatgaaaaacggt
tccatcgccgtgattgacaactcaagaaaagccgtttacggatacgatcagagagctgaagtgttcggatcaaagggaatggt
cgtaacagcaaacgattccgcatcctcagccgtgatcagtaatgcggacggagtgacaggagaaaaacctctttacttcttcct
ggaacgctacatggcagcctatgcaaaagaaatcaaatgctttatcgacgccatcgaaaaaaacacagacacccctcttgg
agttatggacggtcttgagccagtacttatggggcttgcggctaaaaaatcattagaagaacaccgcccagtaaaaatttctga
aattacagtatag
Amino acid sequence
(SEQ ID NO: 2)
MVNVGIIGAGRIGRVHVESICTQVGNAKVKTLADPFMSEDTAVWAKEMGVENTTKDYKDIL
NDPQIDAVLICSSTDTHSPISVEAIEAGKHVFCEKPIDHDVQKIQEVVDALKKSDVKYQVGF
NRRFDHNFEAIRDAVAAGKIGETQIIKVTSRDPEPPSADYVKVSGGMFLDMTIHDFDMVRF
LAGCDAEEVYVEAAVLVDPAIGEAGDVDTAVITLKMKNGSIAVIDNSRKAVYGYDQRAEVF
GSKGMVVTANDSASSAVISNADGVTGEKPLYFFLERYMAAYAKEIKCFIDAIEKNTDTPLGV
MDGLEPVLMGLAAKKSLEEHRPVKISEITV
Myo-inositol 2-dehydrogenase 2 - AR1Y2_0342
Nucleotide sequence
(SEQ ID NO: 3)
atgaacaaagtaagagtaggtattgctggtttaggaagattagggaaattacatgcagagaatatcgcattcaagattccaaac
gcggaacttacagcggcctgttcgatcgcgccagaggaattggaatatgccacagaacatttcggggtaaccgatgtttataca
gatttttatgagatgttagataaagcagacattgatgtcgtggcggtggtgaccacgagcggagaacactgctggcagatcaaa
gcagcattggacgctggaaaacatgttttttgcgataaacctctcggggttacggtcaatgaatgtaaaacagcggaggccgc
ggtggaacgccacccagagcttacattttttcttggatttatgcgccgttatgacccatcctacgcttatgcgaaaaagaaaatcc
aggagggagccattggtaagccgtatatggtaaaagcggcagggatcgacccggaagctctggtggagggagcagtaaaa
tttgcagccaccagcggcggtatttttatcgacatggcaatccatgatatcgatttaatgcgctggcttttggaatccgatccggtgg
aagtctacgctgcaggggccacgtttaaacatcccgaattcagggaggcaggggatgacgagacgggggcagccatgtac
aaatgtgagaacggtgcactgggatttgtacatgttgggcgtacagcggcccatggatatcatgtggagacagagatcatcgg
aacagagggaagcctgagaatcagtcctgtaccggaaaagaacctgtgtatgatctacgatgagaacggagctgtgacaga
atgtgtcagcggttttgcagaacggtttgcagaggcatacctgttggaaatgaaggaattcatcaactgtgtccagagcggcac
aaagccggatgtgaccgtatatgacggcacaaaatccacacagatcggttttgccacaacagaagcgtggaaaaagggctg
tactgcaaagatccagtactag
Amino acid sequence
(SEQ ID NO: 4)
MNKVRVGIAGLGRLGKLHAENIAFKIPNAELTAACSIAPEELEYATEHFGVTDVYTDFYEML
DKADIDVVAVVTTSGEHCWQIKAALDAGKHVFCDKPLGVTVNECKTAEAAVERHPELTFFL
GFMRRYDPSYAYAKKKIQEGAIGKPYMVKAAGIDPEALVEGAVKFAATSGGIFIDMAIHDID
LMRWLLESDPVEVYAAGATFKHPEFREAGDDETGAAMYKCENGALGFVHVGRTAAHGY
HVETEIIGTEGSLRISPVPEKNLCMIYDENGAVTECVSGFAERFAEAYLLEMKEFINCVQSG
TKPDVTVYDGTKSTQIGFATTEAWKKGCTAKIQY
Inosose dehydratase - AR1Y2_0345
Nucleotide sequence
(SEQ ID NO: 5)
atgtttaacaaagaaaaagtaaaattaggaattgccccgatcgcttggaccaacgatgatcttccggatcttggaaaagaaaa
cacctttgagcagtgtgtcagtgaaatggcattggcagggtacactggttccgaagtaggaaataaatatccaaaggacccag
aggttctaaaaaaggccctgaaactccggggaatggagatctgcaatcagtggttctcatcatttttgatcacaaagccatttga
agaggtggaaaaagagttccgcgctcaactgagtttcttaaaggctgtgggagcaaagatcattggtgcttctgaacagagcc
acagtgtacagggtatgctggatactccgatctttggacataagtatgtgatgaatgacgaagagtgggaaaccttctgtacggg
aatgaataagttaggaaaaatcgcaaaagaagaatacggcatcagcttaaccttccaccatcatatgggaactgtggtacag
gacccggatgaagtggaaagaatgatggcaaacacagatcctgaatatgtgaatctgttatttgatacagggcattttacatact
gcggggcagatcctcttgaaatggtgaaaaaatatgtggaccgcatcaaacatgtccatcttaaggatatccgtccggaaatcg
tagaaaaagtgaaagcagaaaacttaagcttcttagatggtgtgcgtatgggggcgtttacggtaccgggtgacggatgtattg
attttgatccgatctttaaggtgcttgaagattcaggatatgaaggatatatgctggtagaggcagaacaggatccggccaaggc
aaatccgcttgaatatgccatgaaggcaagaaaattcatcgctgagaaaactggattataa
Amino acid sequence
(SEQ ID NO: 6)
MFNKEKVKLGIAPIAWTNDDLPDLGKENTFEQCVSEMALAGYTGSEVGNKYPKDPEVLKK
ALKLRGMEICNQWFSSFLITKPFEEVEKEFRAQLSFLKAVGAKIIGASEQSHSVQGMLDTPI
FGHKYVMNDEEWETFCTGMNKLGKIAKEEYGISLTFHHHMGTVVQDPDEVERMMANTDP
EYVNLLFDTGHFTYCGADPLEMVKKYVDRIKHVHLKDIRPEIVEKVKAENLSFLDGVRMGA
FTVPGDGCIDFDPIFKVLEDSGYEGYMLVEAEQDPAKANPLEYAMKARKFIAEKTGL
Epi-inositol hydrolase - AR1Y2_1105
Nucleotide sequence
(SEQ ID NO: 7)
atgagtacaaccagaatgacagtagcacaggctcttgtaaaattccttgacaatcagtatgtggaatttgacggggaagaaac
aaaatttgtagaaggtttctttactatcttcggacacgggatcgccgttggtctcggagaggcattggacagcaatcctgggagct
taaaggttcttcagggaagaaatgagcagggaatgtgccacgtagcgacagcttacgcaaaacaaaatagaagaaggaa
gatcattccttgtgcatcttctgtaggacccggagcggcaaatatggtgacagcagcggctacagctacagtgaacaatattcc
gcttttggtagtgccttctgacacatttgccacaagacagccggacccggtgctccagcagttagaaaagagtgacagtctctct
acaacaacaaacgacgcattcaaaccggtttgtaaatattgggataggatcatccgcccggaacagctgatgacagccatgg
tcaacgctatgcgtgttctcacagatccgtcagagacaggcgctgtgtgcattgcactctcccaggatgtagaaggagaatctta
tgatttccctgactatctgtttgaaaaacgtgtacataggatcacaagaccttgcgcggtaaaagaagagatcgaggaccttgta
aaagtcattaaagaagcgaaaaatccgttagtgatcgtgggcggaggtgtgaagtattctgaggcaggagaagctgtagaaa
agttctgcgaagactttaacatcccgttcggagagagccaggcaggaaaaagtgcctgcaagtcaagccacccttactgctta
ggaggaatcggcgtcacaggaaccctggctgccaataccatcgctgatgaagctgatgtggtcattgcagtgggaaccagac
tttcagactttacaacaggatctaaaggacagttcaaaaatcctaatgtaaaaatggtgacgatcaacaacaataaatatcatg
catataaaatggatgcagtaaaagctgtcggagacgcgaaagtgacagtggaagctctttctgctagactgaaagaggaag
gatacaagtctgcttacacaacagagatccaggacgcaaaggcagcctgggataaagaaatggaccgccttgcgaatattg
agtatacaggcgatgactttgagccggagatcaaagcgagagatccgcgtacgattcctgaattcgtaaaacttacaggggg
acttatcactcagactgcagcattgtgcaaaatccgtgaggtcattgatgaagacgctaatattgtgacagcaggggggagtctt
ccaagctgtatgcagagaatctggaccacagacaaacgaggcggatatcacgcagaatacggatattcctgcatgggatatg
aagtggcgggagcgctcggcgtaaagatggcagagcctgagcatgaatcctacgtggttgtgggggatgccagctttcagat
gctccacagtgagattatgacagcgatgcaggaaaaacaaaagatcaacatccttgtatttgacaactgcggatttggctgtat
caacaaccttcagatgaatcatggagtgggaagtcttgcaacagagttcagataccgcgatgaaaacaatgagatcaagggt
gatctgatcccggttgactatgcaaaagtcggagaaggatacggactcaagacgtacagtgtaagaacgatcaaagagctg
gaggaagcattgatcgatgctaagaaacaggacgttgccacattgatcgacttgaaggtcattccgaaaacaatgacagacg
gttacaaatcatggtggaacgtaggtattgcgacaacttctgtgaaagaaagcgtgcagaaagcatgtgaaggagtattggaa
ggccgcaaaaacgcaagacagtattag
Amino acid sequence
(SEQ ID NO: 8)
MSTTRMTVAQALVKFLDNQYVEFDGEETKFVEGFFTIFGHGIAVGLGEALDSNPGSLKVLQ
GRNEQGMCHVATAYAKQNRRRKIIPCASSVGPGAANMVTAAATATVNNIPLLVVPSDTFA
TRQPDPVLQQLEKSDSLSTTTNDAFKPVCKYWDRIIRPEQLMTAMVNAMRVLTDPSETGA
VCIALSQDVEGESYDFPDYLFEKRVHRITRPCAVKEEIEDLVKVIKEAKNPLVIVGGGVKYS
EAGEAVEKFCEDFNIPFGESQAGKSACKSSHPYCLGGIGVTGTLAANTIADEADVVIAVGT
RLSDFTTGSKGQFKNPNVKMVTINNNKYHAYKMDAVKAVGDAKVTVEALSARLKEEGYKS
AYTTEIQDAKAAWDKEMDRLANIEYTGDDFEPEIKARDPRTIPEFVKLTGGLITQTAALCKIR
EVIDEDANIVTAGGSLPSCMQRIWTTDKRGGYHAEYGYSCMGYEVAGALGVKMAEPEHE
SYVVVGDASFQMLHSEIMTAMQEKQKINILVFDNCGFGCINNLQMNHGVGSLATEFRYRD
ENNEIKGDLIPVDYAKVGEGYGLKTYSVRTIKELEEALIDAKKQDVATLIDLKVIPKTMTDGY
KSVWVNVGIATTSVKESVQKACEGVLEGRKNARQY
5-deoxy-glucuronate isomerase - AR1Y2_1106
Nucleotide sequence
(SEQ ID NO: 9)
atgggtaaagtattcggctatcctgagtttgatgtaaacggggaaaaggttttaacaacatatgacaatgattataaagaaatgct
gatggacatccgcgtcttccgcttaaaagcgggagatgtgaagaccttctgcaaagaaaaagaggagaccgcggttcttttact
aaaaggggatgtcgtattctcatttgatgaaacttccaaagaagtcacaagaaaagatgtatttacagaaggaccttgggcagt
acatatctgcacaggagccaaagtcatagtggaagcaaaggcagactctgaaatcttggtacagtccacaatgaatgaccag
acatttgagagtaaattgtatgcgccggaagatgctccttggggatattcctgtgtaggaaagttcggaaatgtggctaaacgcc
gtgtcaataccatctttgaccatgacattgcaccatactccaacatggtattaggtgaagtcttaaatgaccgcggaaactggtca
ggatatcttcctcacagacatccgcagccggaaacttattacttcctgtttgaccatccggaaggatttggggcaagctttgtggg
agaccaggtatttaagagtacagacggaagcttctcagcgattccagggggagaacttcatcctcaggcagtggctccgggat
accaaatgtatacttgctggatgatccgtcatctggaagggaacccatggcttcagacagacagaaatgaagacgaacgcta
cgtttggcttcatgacgcagaattctaa
Amino acid sequence
(SEQ ID NO: 10)
MGKVFGYPEFDVNGEKVLTTYDNDYKEMLMDIRVFRLKAGDVKTFCKEKEETAVLLLKGD
VVFSFDETSKEVTRKDVFTEGPWAVHICTGAKVIVEAKADSEILVQSTMNDQTFESKLYAP
EDAPWGYSCVGKFGNVAKRRVNTIFDHDIAPYSNMVLGEVLNDRGNWSGYLPHRHPQP
ETYYFLFDHPEGFGASFVGDQVFKSTDGSFSAIPGGELHPQAVAPGYQMYTCWMIRHLE
GNPWLQTDRNEDERYVWLHDAEF
5-keto-2-deoxygluconokinase - AR1Y2_1104
Nucleotide sequence
(SEQ ID NO: 11)
atgaagtatgttgaatttgaccagacaagaccaatggatttagtactcatcggaagggtagccatcgattttaacccggtagatta
tttccacccgttatctgagtgcactaccttcaagaagtatgtgggaggatctccggcaaacatcgcagtgggcatcacaaggca
cggaaagaaagccggattcatcggcaaagtttctgatgaccagttcggagagtatgtagtagacttcttcaataaagagggaat
cgatacatcccatattacaaaatgtacgaacggagaaaagcttggacttacctttacagaaattctttccagagaagaaagca
gcatcttaatgtaccgcaactgcatcgcagacttacagttatctgtggatgacattgacgaagaatacataaagaacacaaaa
gcaatcctggtttccggaacagcattggcacagagcccatccagggaagcagtgttaaaagcagtgatgcttgccaagaaaa
atgatacaaagatcatctttgacattgactatagagaatacaactggaaaaattccgatgagatttcaatctactattctttagtagc
cagagaagcagatatcatcatgggatctagagaagaatttgacctgacagaaaaactgataaaaccgggcagaacagaca
aagaatctgcacaggcatggaacactgaaaatgcaaagatcgttgtcatcaaacatggcaaaaaaggatctacagcatata
ccaatgacggaaatgactattcaatcaaaccgttcccggttgaggcgttaaaaggctttgggggcggagacggatatgcatcc
ggcttcttatatggaatatttgaaggatgggatatgatcgagtgtctggaattcggaagtgcggaagcggccatgatggtgagaa
gccattcttgttctgaagaacttccaaaccctcaggaagtcaaagactttatccgcgagacaaaagacaaatatggcgaaatg
attgcgagagtatag
Amino acid sequence
(SEQ ID NO: 12)
MKYVEFDQTRPMDLVLIGRVAIDFNPVDYFHPLSECTTFKKYVGGSPANIAVGITRHGKKA
GFIGKVSDDQFGEYVVDFFNKEGIDTSHITKCTNGEKLGLTFTEILSREESSILMYRNCIADL
QLSVDDIDEEYIKNTKAILVSGTALAQSPSREAVLKAVMLAKKNDTKIIFDIDYREYNWKNSD
EISIYYSLVAREADIIMGSREEFDLTEKLIKPGRTDKESAQAWNTENAKIVVIKHGKKGSTAY
TNDGNDYSIKPFPVEALKGFGGGDGYASGFLYGIFEGWDMIECLEFGSAEAAMMVRSHS
CSEELPNPQEVKDFIRETKDKYGEMIARV
5-keto-2-deoxy-D-gluconate-6 phosphate aldolase - AR1Y2_1054
Nucleotide sequence
(SEQ ID NO: 13)
atggcattagtcaaaatgaaacagttattagagcaggcaaaaaaggaaaacagaggatgcggtgcattcagtgtgggcaat
atggaaatggtcaaaggggcgattcaggcagcggaggagatgaatacaccgatcatcctgcagatcgctgaagtgcggctc
gcccattcaccgcttcatcttatggggccgatgatggtgcaggcggcaaaagaggcaaaggtggatgtggcagttcatctgga
ccacggacttaacattgagactgtaaaaaaagcgctggaactgggatttacctctgttatgctggatgcatccagagattcgtttg
aagaaaatatcagaaaaacaaaagaagttgtcgctctggcagcacagtacggagcaaccgtggaagcggaattagggctg
gtaggcggaagcgaagacggaaagagtgaccatggaatccagtgcaccaagcctcaggacgccaaggtcttctgtgagca
gaccggagtggatgccctggccgttgccattggaaatgcccacggggactatccggtagctccgactttggaatttggtgtattg
gaagacattcacaatattactgccaaaccattggtgcttcacggcggaagcggtattacagatgaggatttcagaaaagccatc
tctcttggaatctgcaaagtgaacattgcaactgcaagctttaacagcctgacaaaagaagtggaaaactatctggagacaga
aggaaaacataattacttcgatatgaacgaagctatggtaaagggtgtctatgacaacgtaaaacgccatatccagatctttaa
ctgcagataa
Amino acid sequence
(SEQ ID NO: 14)
MALVKMKQLLEQAKKENRGCGAFSVGNMEMVKGAIQAAEEMNTPIILQIAEVRLAHSPLHL
MGPMMVQAAKEAKVDVAVHLDHGLNIETVKKALELGFTSVMLDASRDSFEENIRKTKEVV
ALAAQYGATVEAELGLVGGSEDGKSDHGIQCTKPQDAKVFCEQTGVDALAVAIGNAHGD
YPVAPTLEFGVLEDIHNITAKPLVLHGGSGITDEDFRKAISLGICKVNIATASFNSLTKEVENY
LETEGKHNYFDMNEAMVKGVYDNVKRHIQIFNCR
Glyceraldehyde-3-phosphate keto-isomerase - AR1Y2_1336
Nucleotide sequence
(SEQ ID NO: 15)
atgcgtaaaacaatcgtagcaggaaactggaaaatgaacaaaacacctagcgagacagtagctttagttaatgaattaaagc
cattggtagcaagcgaggtttcagatgttgtcttctgtgtgccggccatctgtattccggcagcactggaagctgcaaaaggaagt
aacatcaacatcggcgctgagaacatgtattttgaagagagcggcgcttacacaggcgagatcgctccgaatatgttaacaga
cttaggtgtgaaatatgttatcatcggacattctgagagaagagaatattttgcagaaacagatgagacagtgaataaaaaagt
cttaaaagcttttgaacatggaattactccaatcgtttgctgcggtgagacacttgctcagagagagcagggtgtgaccatcgatt
tcgtacgccagcagatcaagatcgcattccagggtgtgactgctgaccaggcaaagacagctgtcattgcatacgaaccgatc
tgggctatcggaacaggaaaagttgcgacgacagaacaggctcaggaagtatgtaaagcgatccgtgagtgcatcgctgaa
gtatacgatcaggcaacagcggacgctattcgtatccagtacggcggttctgtatccgctgcaagtgcaccagaattatttgcac
aggaagacatcgatggaggattagtcggtggagctgctttaaaaccagactttggtaagatcgtgaattacaaataa
Amino acid sequence
(SEQ ID NO: 16)
MRKTIVAGNWKMNKTPSETVALVNELKPLVASEVSDVVFCVPAICIPAALEAAKGSNINIGA
ENMYFEESGAYTGEIAPNMLTDLGVKYVIIGHSERREYFAETDETVNKKVLKAFEHGITPIV
CCGETLAQREQGVTIDFVRQQIKIAFQGVTADQAKTAVIAYEPIWAIGTGKVATTEQAQEV
CKAIRECIAEVYDQATADAIRIQYGGSVSAASAPELFAQEDIDGGLVGGAALKPDFGKIVNYK
Glyceraldehyde-3-phosphate dehydrogenase - AR1Y2_1334
Nucleotide sequence
(SEQ ID NO: 17)
atggcagtaaaagtagcgattaatggatttggacgtattggacgtcttgcattcagacagatgttcggagcagaaggttatgaag
ttgtagcaatcaacgatttaacaaatcctaagatgttagcacatttattaaagtatgattctgcacagggaagatatgcattaggcg
acaaggtagaagcaaaagaatcttccatcgtagttgacggaaaagaaatcgaaatctacgcagaagcagacgcaacaaa
acttccttggggagaattagatgttgatgttgtattagaatgtacaggattctacacatctaaagacaaagcatccgcacacgttc
aggcaggagctaagaaagttgttatttctgcgccagctggaaatgatcttccaactatcgtatataatgtaaaccacgatacattg
acaaaagaagacacagtcatctcagcagcatcttgtacaacaaactgcttagcacctatggctgatgcactgaacaagtatgc
accaatccagtctggtatcatgttaacagtacatgcttacacaggagatcagatgactcttgacggacctcagagaaaaggtga
cttaagaagagcacgtgcagctgcagtcaacatcgtacctaactctacaggtgctgcaaaagctatcggattagttatcccaga
attaaacggaaaattaatcggatctgctcagcgtgtaccggttcctacaggatcaacaacacttttaacagcagttgttaaaggt
gacaatgtaactgttgaaggaatcaacgcagctatgaaagcagcagcaacagagtctttcggatacacagaagaagaatta
gtatcttctgacatcatcggaatcaaatatggttctttattcgacgcaactcagacaatggttactgaagttggtgacggattatatg
aagtacaggttgtttcttggtatgacaacgaaaactcatacacaagccagatggttagaacaatcaaatactttgcagaattagc
ataa
Amino acid sequence
(SEQ ID NO: 18)
MAVKVAINGFGRIGRLAFRQMFGAEGYEVVAINDLTNPKMLAHLLKYDSAQGRYALGDKV
EAKESSIVVDGKEIEIYAEADATKLPWGELDVDVVLECTGFYTSKDKASAHVQAGAKKVVIS
APAGNDLPTIVYNVNHDTLTKEDTVISAASCTTNCLAPMADALNKYAPIQSGIMLTVHAYTG
DQMTLDGPQRKGDLRRARAAAVNIVPNSTGAAKAIGLVIPELNGKLIGSAQRVPVPTGSTT
LLTAVVKGDNVTVEGINAAMKAAATESFGYTEEELVSSDIIGIKYGSLFDATQTMVTEVGDG
LYEVQVVSVVYDNENSYTSQMVRTIKYFAELA
Phosphoglycerate kinase - AR1Y2_1335
Nucleotide sequence
(SEQ ID NO: 19)
atgttaaacaagaaatcagtagatgatatcaacgtaaaaggaaaaaaagtcttagtgcgctgtgacttcaacgttccgcttcag
gatggaaagatcacagatgagaaccgtcttgtggcagcacttcctacactaaagaagttaattgctgacggcggaaagctgat
cctctgctcacacttaggaaaaccaaaaggggaaccgaaaccggagttatctcttgcgccagtagcagaaagattaagcga
gttattaggacaggaagtaaagtttgctgcagatgacaacgtggtaggaccgaatgcaaaagctgcggtagaaggaatgca
ggacggggatgtcattttacttcagaatacacgctacagagcagaagagacaaagaatggagaagagttcagcaaagaact
tgcaagtctctgcgatgtttttgttaatgatgcttttggtaccgctcatagagctcactgctctaacgtgggagttacaaagttcgtgg
acactgcagttgtaggatatttaatgcagaaagagattgatttcttaggaaatgcggtcaacaatccggaacgtcctatggtcgct
atcttgggaggagcaaaggtttcctctaagatctctgtgatcaataacctgctagacaaagtcgatactttgatcatcggcggag
gtatggcatatactttcatgaaagcattgggagaagaagttggagattcccttctggaagctgactacttagactacgcaaaaga
aatgatggacaaggcaaaagaaaaaggtgtgaacttactgattcctgttgacactgtagttgctgatgacttctcaaatgacgcc
aacatcaaaactgttgagagaggcggaatcgaagcaggatggcagggacttgacatcggaccaaagacaagagaactgtt
ttcaaatgcactgaaagatgccaagactgtagtatggaacggaccgatgggatgctttgagatgccgaactttgcagcaggaa
ccgttgcagttgcagaagcactggcagagatcgacgctacaactatcattggcggaggagattccgcagcggcagttaacca
gctcggattcggagataagatgactcatatctctacaggtggtggagcatcactggaattcctggaaggaaaagaattaccag
gcgttgcagcggcaaacgataaataa
Amino acid sequence
(SEQ ID NO: 20)
MLNKKSVDDINVKGKKVLVRCDFNVPLQDGKITDENRLVAALPTLKKLIADGGKLILCSHLG
KPKGEPKPELSLAPVAERLSELLGQEVKFAADDNVVGPNAKAAVEGMQDGDVILLQNTRY
RAEETKNGEEFSKELASLCDVFVNDAFGTAHRAHCSNVGVTKFVDTAVVGYLMQKEIDFL
GNAVNNPERPMVAILGGAKVSSKISVINNLLDKVDTLIIGGGMAYTFMKALGEEVGDSLLEA
DYLDYAKEMMDKAKEKGVNLLIPVDTVVADDFSNDANIKTVERGGIEAGWQGLDIGPKTR
ELFSNALKDAKTVVWNGPMGCFEMPNFAAGTVAVAEALAEIDATTIIGGGDSAAAVNQLG
FGDKMTHISTGGGASLEFLEGKELPGVAAANDK
Phosphoglycerate mutase - AR1Y2_0899
Nucleotide sequence
(SEQ ID NO: 21)
ttgaagttatatatgatgaggcacggagaaactccatggaataaagtgcggaggattcaggggcagtcagacgtggatctga
gtgaagcgggcagggaagcagccaggctcaccaggagagcactgaaagatttccattttgatgcggcctacacgagcccg
ctgagcagagcgagagaaacaggggagatcatcctggagggcagagggatccctctgctggaggatgaacggcttaagg
aggccaacttcggaccttatgaaggggcacacatcaatgaattatatgaaagagaagaaccgattctggatttctttgaccggc
cggaacagatggcctatattgaaggagcggagactccggctcaggtagtagaaagatccaggatatttttagaggaatttatac
ttccgatggaaaaagaagcagaaagtgtgatcttatttacccacggagctttcatccacgggatgctgactaatatgtatcaaag
agagatcagggacttttggaaaggaccgaagcttaagaattgttctatcagtacggctgaagtcagggacggtcagttccgcat
tttgagtgaggctgaggtcttttactga
Amino acid sequence
(SEQ ID NO: 22)
MKLYMMRHGETPWNKVRRIQGQSDVDLSEAGREAARLTRRALKDFHFDAAYTSPLSRAR
ETGEIILEGRGIPLLEDERLKEANFGPYEGAHINELYEREEPILDFFDRPEQMAYIEGAETPA
QVVERSRIFLEEFILPMEKEAESVILFTHGAFIHGMLTNMYQREIRDFWKGPKLKNCSISTA
EVRDGQFRILSEAEVFY
Enolase - AR1Y2_0982
Nucleotide sequence
(SEQ ID NO: 23)
atggattatttagagattgaaaaggtcataggaagggaaattattgattccaggggaaatcctacagtggaagcagaagttctttt
agccggcgggacaatcggacggggcacagcacccagcggagcttcaactggagaatttgaggcgttggaattaagagacg
gagataaggagcgttttggaggaaaaggtgtgtccaaggcagttcataatattaatacgatcattaacgatactttaaaagggat
ggatgcatccgatacttacgcggtggacagagcgatgatcagcgtagacggaacaaaggataaatcaaagcttggggcca
atgcgatccttgcggtttcgattgcatgcgcaaaggcggcatcggcaggcctggatctgccattataccgttttctgggcggtgtta
atgccaaccgtcttccggtgccgatgatgaacattttaaacggaggtgcccatgcggccaacaccgtcgatgtacaggagttta
tgatcatgcctgtgggagcagaaaatttcagggaagcactccggtggtgtacggaagtattccacgccttggcagctttgctgaa
atccaaagggctggcaacctccgtgggtgatgaaggcgggtttgcgcctgaccttgcaagtgatgaggaagccatcgaatata
ttttggaagctgttcaggcagcgggatatgagccgggcagtgattttgttctggctatggatgctgcttccagcgagtggaaaggc
gcgaacaaaggtgaatatattcttcctaagagtggcaagaaatttacctcagaagaactggtggaccactggaaatcactttgt
gagaaatatccgatcagctccattgaggatggattggatgaggaagactgggacggctggaaatacatgacccaggagcttg
gagagaaaatccagctggtgggagatgacttatttgtaacgaatacggaacgccttggaaaaggtatcaagatgggatgcgg
aaattctatcttgatcaagctgaaccagatcggttctgtgtcggagacattggaagctattaagatggcccataaagcgggttac
acagcggtcacatcccaccgttcaggggagacagaagacacgaccatcgcagatctcgctgttgcacttaataccggacag
atcaaaaccggtgcaccgagcagaagtgagagggtggcaaaatataatcagcttctgaggattgaagaggaactggggga
aagtgccgtatacccgggagtcggaacattttag
Amino acid sequence
(SEQ ID NO: 24)
MDYLEIEKVIGREIIDSRGNPTVEAEVLLAGGTIGRGTAPSGASTGEFEALELRDGDKERFG
GKGVSKAVHNINTIINDTLKGMDASDTYAVDRAMISVDGTKDKSKLGANAILAVSIACAKAA
SAGLDLPLYRFLGGVNANRLPVPMMNILNGGAHAANTVDVQEFMIMPVGAENFREALRW
CTEVFHALAALLKSKGLATSVGDEGGFAPDLASDEEAIEYILEAVQAAGYEPGSDFVLAMD
AASSEWKGANKGEYILPKSGKKFTSEELVDHWKSLCEKYPISSIEDGLDEEDWDGWKYM
TQELGEKIQLVGDDLFVTNTERLGKGIKMGCGNSILIKLNQIGSVSETLEAIKMAHKAGYTA
VTSHRSGETEDTTIADLAVALNTGQIKTGAPSRSERVAKYNQLLRIEEELGESAVYPGVGTF
Pyruvate-flavodoxin oxidoreductase - AR1Y2_3042
Nucleotide sequence
(SEQ ID NO: 25)
atggcaagaaaaatgaaaaccatggatggtaacacagctgcagcacacgtgtcttacgcttttactgagacagctgctatctac
ccaattacaccatcctcaccaatggcagaatcaacagaccagtgggcaacacagggaaagaaaaatattttcggacatcct
gtaaaattagttgaaatgcagtcagagggcggagcagcaggaactgttcacggatctcttgcagcaggcgcattgacaacta
catacactgcatctcagggattattactgatgattcctaatatgtacaagatcgcaggtgaattaatgccttgtgtatttgatgtatctg
cacgtgcaatcgcatctcatgcactgaatatctttggtgatcactctgacgtttacgcttgtcgccagacaggttttgcaatgctttgc
agctccagcgttcaggaagttatggacttaggcgctgttgctcatttatctacaatcaagggaagcgttccagttctccatttcttcg
atggattcagaacatctcatgagatccagaagattgaaatgtgggacaacgaagatcttgcagatatggtagattacgatgctct
tgaaaagttcagagcaaatgctttgaatccagaacatccggttctccgcggatctgctcagaatccagatatcttcttccaggca
agagaagcatgcaacaaatactacgatgcattcccggcaatcgtcgaagattacatgaaccaggttaatgcaaagatcggta
cagattataaattattcaactactacggagctgctgatgctgaatctgttatcattgcaatgggatctgtatgtgagactatcgatga
gaccatcgattacttaacagcaaaaggcgaaaaagtcggtgttgttaaagtaagactttacagaccattcgtagctgaaaaatt
actcgaagttatcccagagactgttaaaacaatcaacgttttagacagaacaaaagaaccgggatctcttggagagccattat
acttagacgttgtttctgcattaagaggaacaaaattcgagcaggtaacaatcaacggaggacgttacggattaggatccaaa
gatacaactcctgctgatatcatctctgtattcaataacaaagataagaaagaattcacagttgcgatcaacgatgatgtgacaa
acttatctcttgaaagaggagaagctgtttcttctacaccagaaagcacagtaagctgtaagttctggggtcttggagcagatggt
accgttggtgctaacaagaactccatcaagatcatcggtgaccacactgacaaatatgctcaggcatactttgattatgattcca
agaaatccggtggtgttacaatttcccacttacgtttcggcgatgacccgatcaaagcaacttacctgatcaacaaagctgatttt
gttgcatgtcactgtgctccgtacatgacaaaatacaacatggttcaggacttaaaagatggcggaacattcttgttaaactgtag
ctggccggtagaagaaatcggagatcatcttccaggacaggtaaaagcttacatggcaaaacacaacatcaagttctatgtg
atcgatggtatcaagatcggtaaagagattggacttggcggacgtatcaatactgtacttcagtctgcattcttcaaattatctgga
attattccggaagcagacgcgatccagtatatgaaagacgctgctactgcttcttacagcaagaagggtgatcacatcgttaag
atgaatcatgacgcgatcgaagctggtgctaagagtgttgtagaagttccggttcctgctgaatgggctgactgcggagaagaa
agcttattacatccagtatccggagatagaaaagatcttgttgattatgttaacaacattcagaacgctgtcaatggacagattggt
aacagccttaaggtaagcgacttagttccttacgctgacggtcatatgcctcagggatctgctgcatacgaaaaacgtggagttg
ctgtagacgttcctacatggaatccggacaactgtatccagtgtaacttctgtgcatatgtatgtcctcatgcagttatccgtcctgtt
gccatgacagaagcagaacttgctgcagcaccggaagctacaaaatctcttccgatgacgggaatgccagattacaaatttgt
aatgacaactactgttcttgactgtacaggatgtggttcttgtgcaaatgtctgcccaggtaagaaaggcgagaaagctcttaca
atggcaccactcgacagcatgagagatcagcaggaagtttacaactatggacagtctttacctataaaagaagatatctttgac
aagtttaaaccagaatctgttaagggaagccagttcaagaaacctctccttgagttctcaggagcttgtgcaggatgtggagag
actccatatgcgaaactgatcactcagttattcggagacagaatgtacattgccaatgcgacaggatgttcttctatctggggtggt
tcttcacctgcaacaccttacacagtaaacagcgaaggacagggaccggcttggtctaactcactgttcgaagacaatgctga
gtttggtttaggtatgttcacaggacagaatactttaagagagcagacaaaagaaaaggttgcagcattagcagaatctacag
ataatgcagatgtaaaagcagcagcggctgaatacttagatacttacacagacggtaagaaaaacggagcagctactaaga
aattagttgcagcactggaagcttgcggatgtgaagaaggaaaagaaatccttaagatgaaagattacttatctaagaaatctc
agtggatcttcggtggtgacggatgggcttacgacatcggctttggcggtgttgaccatgtacttgcaagtggtgaagatgttaata
tctttgtattcgatacagaggtttactctaacaccggcggacaggcatccaaatctacaccggcaggtgcgatcgctcagttcgc
agcagcaggtaaggctgtgaagaaaaaagaccttgcagcaatcgcaatgagctacggatatgtatatgttgctcagatctctat
gggtgctgattacaatcagacagtcaaagcgatcgcagaggcagaagcttatccaggaccatcactgatcatcggttatgctc
catgtatcaaccacggtatcaagggtggaatgagcattgctcagtctgaagaaaagaaagcagttacatcaggatactggaa
cttattccgtttcaaccctgaattagctgaagaaggaaagaatccattctcattagattctaaagagcctacaacagagtacaatg
acttcatcatgggtgaggttcgttacaacgctcttgctcgccagaatcctgagagagctaaggagctgtttgagcaggcagaaa
agaatgccaaagaaaaatatgctcgtttgaaagaattagcagataaataa
Amino acid sequence
(SEQ ID NO: 26)
MARKMKTMDGNTAAAHVSYAFTETAAIYPITPSSPMAESTDQWATQGKKNIFGHPVKLVE
MQSEGGAAGTVHGSLAAGALTTTYTASQGLLLMIPNMYKIAGELMPCVFDVSARAIASHAL
NIFGDHSDVYACRQTGFAMLCSSSVQEVMDLGAVAHLSTIKGSVPVLHFFDGFRTSHEIQ
KIEMWDNEDLADMVDYDALEKFRANALNPEHPVLRGSAQNPDIFFQAREACNKYYDAFPA
IVEDYMNQVNAKIGTDYKLFNYYGAADAESVIIAMGSVCETIDETIDYLTAKGEKVGVVKVR
LYRPFVAEKLLEVIPETVKTINVLDRTKEPGSLGEPLYLDVVSALRGTKFEQVTINGGRYGL
GSKDTTPADIISVFNNKDKKEFTVAINDDVTNLSLERGEAVSSTPESTVSCKFWGLGADGT
VGANKNSIKIIGDHTDKYAQAYFDYDSKKSGGVTISHLRFGDDPIKATYLINKADFVACHCA
PYMTKYNMVQDLKDGGTFLLNCSWPVEEIGDHLPGQVKAYMAKHNIKFYVIDGIKIGKEIG
LGGRINTVLQSAFFKLSGIIPEADAIQYMKDAATASYSKKGDHIVKMNHDAIEAGAKSVVEV
PVPAEWADCGEESLLHPVSGDRKDLVDYVNNIQNAVNGQIGNSLKVSDLVPYADGHMPQ
GSAAYEKRGVAVDVPTWNPDNCIQCNFCAYVCPHAVIRPVAMTEAELAAAPEATKSLPMT
GMPDYKFVMTTTVLDCTGCGSCANVCPGKKGEKALTMAPLDSMRDQQEVYNYGQSLPIK
EDIFDKFKPESVKGSQFKKPLLEFSGACAGCGETPYAKLITQLFGDRMYIANATGCSSIWG
GSSPATPYTVNSEGQGPAWSNSLFEDNAEFGLGMFTGQNTLREQTKEKVAALAESTDNA
DVKAAAAEYLDTYTDGKKNGAATKKLVAALEACGCEEGKEILKMKDYLSKKSQWIFGGDG
WAYDIGFGGVDHVLASGEDVNIFVFDTEVYSNTGGQASKSTPAGAIAQFAAAGKAVKKKD
LAAIAMSYGYVYVAQISMGADYNQTVKAIAEAEAYPGPSLIIGYAPCINHGIKGGMSIAQSE
EKKAVTSGYWNLFRFNPELAEEGKNPFSLDSKEPTTEYNDFIMGEVRYNALARQNPERAK
ELFEQAEKNAKEKYARLKELADK
3-oxoacid CoA transferase - AR1Y2_1115
Nucleotide sequence
(SEQ ID NO: 27)
atgggaaaagttaaaattatcacagcagatcaggctgctgccctggtggaggatcatacaacgatcacaaccagcggatttgt
tgccagcggaatgccggaagctctcacaaaagctttggaaaaaagattcaaagaaaccggatcccccaaaaacctgacttt
gttttatgctgccgcccagggaaaccgtgacggaagcggcgcggatcattttgcccatgaaggtatgacaaaacgtgtcatag
gcggacattggaacatggttcctgcactgggacagatggttctggacaataaaatcgaaggctataatctgcctcagggaaca
ctggcacagttataccgtgccatcgcaggtcacaaacccggcgttatcagtcatgtaggattaaatacatttgctgacccacgca
ttgagggcggtaaattaaatgacattacaaccgaagatatcgtcgatgtagttgaaatcctgggagaagagaaattattgtaca
aatcgttcccgatcaacatcggatttatccgcggtacatatgcagatgaacacggcaacgtaactctgtctcacgaatgtgccac
cacagaggtcacaaccatggctcaggccgtaaagaacagcggcggaaaggtcgttgtacaggtagaaaaggtcgtttcag
acggcaccttagatccaaaattagtcaagatccctggaatttatgtggatgccgttgtggaagtagaagacatgaaggatcacg
aacaatgcgtgggctgtgattatgatggttccatgaccggagacttccgggttccgttaagcagtcttgagtacccgcctctttccg
ccaagaagatcattggacgcagagcagccatggaactgacagaaaacacggttgtaaatctaggcattggtattccagagta
catctccatggtagccaacgaagaaggcattggagattacatgactctgactgtagaggccggaccaatcggcggtgtacctc
agggaggagctaaattcggaggcgctgtcaatgctgaatgtatcctagatcagccgtaccagtttgatttctatgacggcggcg
gagttgactatgcattcttaggactcgcacaggcagataaagacggtaatatcaacgtaagcaagttcggtccaaggattgcg
ggctgcggcggtttcgtcaatatcacccagaacgcaaaacgctgctacttctgcggaacttttactgcaggaggtctgaagaca
tccgtaaaagacgggaaacttatcattgaccaggaaggaaaatcaaacaaattcctggataccgtagaacagatcaccttca
gcggtgaatacgcaaataaagtcggccagccggttctctatatcacagaacgtgctgtattcgaactcagaaaggacggcgtc
tatctgacagaggttgcccctggtatcgatatccagacacagatcatcgatcatatgggatttgctccaaagatggaaggaacg
ccaaagttcatggatgaaagaatcttcaaagatgagctgatgggcctgaagcacgattaa
Amino acid sequence
(SEQ ID NO: 28)
MGKVKIITADQAAALVEDHTTITTSGFVASGMPEALTKALEKRFKETGSPKNLTLFYAAAQG
NRDGSGADHFAHEGMTKRVIGGHWNMVPALGQMVLDNKIEGYNLPQGTLAQLYRAIAGH
KPGVISHVGLNTFADPRIEGGKLNDITTEDIVDVVEILGEEKLLYKSFPINIGFIRGTYADEHG
NVTLSHECATTEVTTMAQAVKNSGGKVVVQVEKVVSDGTLDPKLVKIPGIYVDAVVEVED
MKDHEQCVGCDYDGSMTGDFRVPLSSLEYPPLSAKKIIGRRAAMELTENTVVNLGIGIPEY
ISMVANEEGIGDYMTLTVEAGPIGGVPQGGAKFGGAVNAECILDQPYQFDFYDGGGVDYA
FLGLAQADKDGNINVSKFGPRIAGCGGFVNITQNAKRCYFCGTFTAGGLKTSVKDGKLIID
QEGKSNKFLDTVEQITFSGEYANKVGQPVLYITERAVFELRKDGVYLTEVAPGIDIQTQIIDH
MGFAPKMEGTPKFMDERIFKDELMGLKHD
Acyl-CoA dehydrogenase - AR1Y2_1117
Nucleotide sequence
(SEQ ID NO: 29)
atgaattttgaattaacagagcagcaggcagccattcaggaaacagcaaggaactttgctcagacagaactgcagccaggt
gtgctggagagagatgcaaacagtgagtttccggtagatctttataagaagatgggggatatgggcctgatcggtcttccatatc
cgaaagaagtcggaggacagggagccgattatctttcttatgcactggcagtggaagagatctcaaaagtagatgcctctgtg
ggtatttcctattctgtttccacctctctgtatggcggaagcattatgaactctgatgcttccgttgagaaaaagaatgaatttctagca
ccggtactgtccggacagcatttcggatcctttggactcacagagcccaatgccggatcagacgcaggcggatgtgtcactgtg
gctgaaaaagatggggacgaatacatattaaatggtatgaagtgttttaacaccaacggacctttggccgattatacagcggtat
atgccctgacagaacctgagaagaaagcaaagggtctttcctgctttgtagtaaaaaaaggaactcctggattttctgtaggca
aggtggaggataagatgggaatccgctctgcgcaggtctctgagatgatcctggaaaatgtcagagtacctgcagagaatatg
gtcgttccatctggggacggatttaagctggccatgaagactttggatggaggacgcatcggcgttgccgcgcaggggcttggt
atcgcggagggcgcatttgagatcgcaaaagaatacttaaagagcagagaacagttcggcaaaccattatataaaaaccag
tatcttgcatttaagatggcggaacttgaaatggagatcgacgctgcaagatacatgctttacaaagcggctacagataaacac
gagggaagatcttactcaatcccggcagcaaaggcaaaatacctttgtacagaggccgctatgcatgtgacaacagaagca
gtacagatgttaggtggaaatgggtatatgaagggttaccatgtagaacgtatgatgagagatgcgaagatcactcagatctat
gagggaaccaatgaaatccagaagctcgttgtaagcggagctatcttcagatag
Amino acid sequence
(SEQ ID NO: 30)
MNFELTEQQAAIQETARNFAQTELQPGVLERDANSEFPVDLYKKMGDMGLIGLPYPKEVG
GQGADYLSYALAVEEISKVDASVGISYSVSTSLYGGSIMNSDASVEKKNEFLAPVLSGQHF
GSFGLTEPNAGSDAGGCVTVAEKDGDEYILNGMKCFNTNGPLADYTAVYALTEPEKKAK
GLSCFVVKKGTPGFSVGKVEDKMGIRSAQVSEMILENVRVPAENMVVPSGDGFKLAMKTL
DGGRIGVAAQGLGIAEGAFEIAKEYLKSREQFGKPLYKNQYLAFKMAELEMEIDAARYMLY
KAATDKHEGRSYSIPAAKAKYLCTEAAMHVTTEAVQMLGGNGYMKGYHVERMMRDAKIT
QIYEGTNEIQKLVVSGAIFR
Enoyl-CoA hydratase - AR1Y2_1116
Nucleotide sequence
(SEQ ID NO: 31)
atgaacaatttattaatggaagtagaaaacgaagtcgcagttgtaacaatcaacagaccaaaatcattaaacgctttaaacagt
gaaactttagctgagttagaccagtgctttacagagatttcaggacgcaaggacatcagagtggtgatcctcacaggatcagg
cgaaaaatcctttgtagctggtgctgatatctctgaaatggtcaatgcaactcctgcagaaggaagacagatgggtcttttggca
aaagaagcatttcttaagttagagacaatgcctcaggtaaccatcgctgccgtcaacggttacgctctgggaggcggatgcga
gatctccatggcatgtgatatccgtgtagctgcggaaaacgcaaaattcgcacagccagaaacaggacttggaattcttccgg
gattcggcggaacacagcgtctctcccgtttagtaggaaaaggacgcgcaaaggaattgatctttacatgtgaccagatcgatg
ccgaagaagcttacagaattggtcttgcaaacaaagtagtgcctcaggcggagctgatcgattactgtaagaagatggctgca
aagatcatgtccaaaggaagctacgcaatttcccttgcaaaagaagcgatcaacacaggaatggacacagacttaagcagc
ggtcttacattagaagctgacttattcggacttgcattctccactgctgacaaaaaagagggtatgacagcattccttgaaaaacg
taaagctgatttaaaagatttctag
Amino acid sequence
(SEQ ID NO: 32)
MNNLLMEVENEVAVVTINRPKSLNALNSETLAELDQCFTEISGRKDIRVVILTGSGEKSFVA
GADISEMVNATPAEGRQMGLLAKEAFLKLETMPQVTIAAVNGYALGGGCEISMACDIRVAA
ENAKFAQPETGLGILPGFGGTQRLSRLVGKGRAKELIFTCDQIDAEEAYRIGLANKVVPQA
ELIDYCKKMAAKIMSKGSYAISLAKEAINTGMDTDLSSGLTLEADLFGLAFSTADKKEGMTA
FLEKRKADLKDF
Acryloyl-CoA dehydrogenase - AR1Y2_1050
Nucleotide sequence
(SEQ ID NO: 33)
atggttgaatttaaattgacaaaagaacagctgcttgcacagcagatgttcaaagatttcgcagagacagaaatcaagccaatt
gctagggacatggatgaaacagaagaattcagcatggaactgcttgagaaaatgaaaaaattcggtcttatgggaattccata
ttccaaagaatacggcggagaaggggcagacgttttaacttatacactctgtatggaagagatgtctaagttagatgcttccaca
ggaatcacaatttccgtacatacatcattgtgctgttcctgcatcaatgattttgcttcagaagagcagaaacaaaaatttttacgtc
cgttggttgacggttctaaagtgggatgctttggtcttacagagccgggagcaggtaccgatgccagcggtgtgaagacaaatg
ccgtattggacggcgatgaatatatcttaaacggacagaaggtgttcactacaaactcaggctttgccgatacatttattatttttgc
agttacagacaaaactgcaggtacaagaggtatgtctgcatttatcgtggacaggaatacaccgggattagaagtcagcaag
aatatccctcgtatgggtatccgtgctgcgtcaaactgtgaagttgctttagttgatgtgagagtaccgaaggaaaacctgatcgc
gggcgagggaaaaggatataagatcgctatgtcagcgctggcaggcggacggatcggaatcggcgctcagtccgtaggaa
tcgcacagggtgctttaaacgaggccattgcatatgtaaaggaaagaaaacagttcggaaaaccgatttctaaattccagaat
acacagttcctgatcgcagaactccaagtgaagatcgacgctgcgcgccttatggtatggagagcggcaaaatcaaaagac
gatcatgaaaactatgctcctctggcagcagcgtgcaaactgtttgcatctgacgtggcagtggaagtgacaagagcagccgt
acagcttcttggaggatatggatactccagagaatatccggtagagagaatgtaccgtgatgctaagatcacagagatttacga
aggtacatctgaagctatgaagatgatcgtcggcggcggacttttaagataa
Amino acid sequence
(SEQ ID NO: 34)
MVEFKLTKEQLLAQQMFKDFAETEIKPIARDMDETEEFSMELLEKMKKFGLMGIPYSKEYG
GEGADVLTYTLCMEEMSKLDASTGITISVHTSLCCSCINDFASEEQKQKFLRPLVDGSKVG
CFGLTEPGAGTDASGVKTNAVLDGDEYILNGQKVFTTNSGFADTFIIFAVTDKTAGTRGMS
AFIVDRNTPGLEVSKNIPRMGIRAASNCEVALVDVRVPKENLIAGEGKGYKIAMSALAGGRI
GIGAQSVGIAQGALNEAIAYVKERKQFGKPISKFQNTQFLIAELQVKIDAARLMVWRAAKSK
DDHENYAPLAAACKLFASDVAVEVTRAAVQLLGGYGYSREYPVERMYRDAKITEIYEGTS
EAMKMIVGGGLLR
Acryloyl-CoA dehydrogenase/Etf - Electron transfer flavoprotein, beta subunit -
AR1Y2_1051
Nucleotide sequence
(SEQ ID NO: 35)
atgaaaatcgttgtttgtgcaaaacaagtaccagatacaactgaagtaaaattagatccaaagaccaacactctgatcaggga
tggtgttccgagtatcatcaacccagatgacaaagccggaatcgaagctgccctgcagttaaaggaaaaatgcccgggaag
cactgtgactgttgtttccatgggaccgcctcaggcagatgtggcattgagagaagcacttgctatgggatgtgacgatgcagttc
tggtttctgacagagcattcggaggagcagatacatgggcaacatcttccactatcgcggcagcgatcaaaaaattggattacg
atgtcatcatcacaggacgccaggcgattgacggagataccgctcaggtaggccctcagatcgcagaacatcttggaattcct
caggtgagctatgtcgaagccctgaatgtggaaggggaagacactttgatcgtccagaggcagtttgaagaccgccatcaga
tcattgaagtaaagactccttgcctgatcactgctttggctgaattggcaacacctagatatatgacagtccacggtgcatttgacg
catacagagaaaaagaagtcagcgtctggggacttgcagacttagaagacactgtagaccaggcaaatatcgggttaaaag
gatctcctacaagagtaaagaaatcctttacaaaacaggccaaaggtgcaggaactattttaaaagatttatcagcagacgaa
gctgttgcatccattgttgataaacttcaggaaaagcacatcatttaa
Amino acid sequence
(SEQ ID NO: 36)
MKIVVCAKQVPDTTEVKLDPKTNTLIRDGVPSIINPDDKAGIEAALQLKEKCPGSTVTVVSM
GPPQADVALREALAMGCDDAVLVSDRAFGGADTWATSSTIAAAIKKLDYDVIITGRQAIDG
DTAQVGPQIAEHLGIPQVSYVEALNVEGEDTLIVQRQFEDRHQIIEVKTPCLITALAELATPR
YMTVHGAFDAYREKEVSVWGLADLEDTVDQANIGLKGSPTRVKKSFTKQAKGAGTILKDL
SADEAVASIVDKLQEKHII
Acryloyl-CoA dehydrogenase/Etf - Electron transfer flavoprotein, alpha subunit -
AR1Y2_1052
Nucleotide sequence
(SEQ ID NO: 37)
atgagcagagacgtatatgtattcgctgagcagagagacggcgaacttcagaaagtcggaatcgaattaattggtaaagcaa
gagaattagcagatgatcttggacagcaggtcgttgccgtgctgcttggggcaggcgtaaaagaaaaagcccaggaattagtt
gcctgcggtgcagataaagttgttgtcgtagatgacattatgctggcagattatgtgacagagccatacacaaaggcactgaca
gctgtgattaacgcaaaagatcctgagatcgtgctttatggtgcatcttccatcggacgtgaccttgccccaagagtatctgcaag
gatccatactggattgacagcggactgtactgcacttgccattgatgaagagacaaagctcctcatgatgacccgtccggcattt
ggcggaaatatcatggcgaccatcgtgtgtgaagacttccgtcctcagatggcaacagtccgtccaggagtaatgaaagcctt
ggaaaacgatgcatccagaagcggtgaagtggaagagttcaaagtagaattctctgatgcagacatgaatgtaaaagtccgt
gaaactgtaaagacagcacataaatctgtagacattacagaggctaagatccttgtttccggtgggcgcggaatcggaagcg
ctgacaagttcaaggtgttagaggaactggcggatgtcatggaaggtgaagtgtcttcttccagagcatgtgtagattccggatg
gatcagtgctgaccgccaggtaggacagacaggaaagactgtgcgtccggaattatatctggcctgcggcatctcaggagcg
atccagcatgcagccggtatggagaactcagagtttatcgtagccatcaaccgtgatgaggatgcgccgatctttgatattgcag
acctcggcatcgttggagacttaaataccatcgtaccgaaacttacagaagctgtgaaggcggtaaaaggaaaataa
Amino acid sequence
(SEQ ID NO: 38)
MSRDVYVFAEQRDGELQKVGIELIGKARELADDLGQQVVAVLLGAGVKEKAQELVACGAD
KVVVVDDIMLADYVTEPYTKALTAVINAKDPEIVLYGASSIGRDLAPRVSARIHTGLTADCTA
LAIDEETKLLMMTRPAFGGNIMATIVCEDFRPQMATVRPGVMKALENDASRSGEVEEFKV
EFSDADMNVKVRETVKTAHKSVDITEAKILVSGGRGIGSADKFKVLEELADVMEGEVSSSR
ACVDSGWISADRQVGQTGKTVRPELYLACGISGAIQHAAGMENSEFIVAINRDEDAPIFDIA
DLGIVGDLNTIVPKLTEAVKAVKGK
Inosose isomerase - AR1Y2_1058
Nucleotide sequence
(SEQ ID NO: 39)
atgaaaatcggatttaacgaagcaactgcactggaatgtaaaggacagtctctgatggctgacttagaagcgtgtgaaaaata
cggatttgattacatcgagatcagatttgactgtgtaaaggattatttgaaagaacacacactggaagaactggcggactggttt
aaaaaccatcatttaaaaccatgggcttataataccttgatcttttttaaccagagagacgaagccggagaaaaagagattgac
gaagagactgattttatcattgaagtgtgcaaggccatcggcatgaagatgctgatcactgttccgtcctttgatgtaaaggataa
gagcatcacagagatcaaagaggaagcagtagaacgtctccgttatttatccgataaggtgggagaagatattaagatatcac
tggaattctgcggggcgccgaactgctccatcaaccagtttgggacagcttacgatgtagtgaaagcaacagaccgtgacaat
gtaggggtaacagtggataccttccatttccatgagatgtgttctaagttagaagatttagaagcagcggacggaaagaagatc
tttgcttaccatctgaatgactgtgaggatctgcctctgggatcatgcggtgatgataaacgcctgtggccgggagaaggtgtggt
agatcatgcaggaatcgcagctgccttaaagaagatcggattcgacggggtatgtaccattgaagaattccgtccagaatatta
tgaaatgtcccatgacgagaatgtgaaaaagtctgctgaagtgacaagggatttcgtagcaaaatactttgcataa
Amino acid sequence
(SEQ ID NO: 40)
MKIGFNEATALECKGQSLMADLEACEKYGFDYIEIRFDCVKDYLKEHTLEELADWFKNHHL
KPWAYNTLIFFNQRDEAGEKEIDEETDFIIEVCKAIGMKMLITVPSFDVKDKSITEIKEEAVER
LRYLSDKVGEDIKISLEFCGAPNCSINQFGTAYDVVKATDRDNVGVTVDTFHFHEMCSKLE
DLEAADGKKIFAYHLNDCEDLPLGSCGDDKRLWPGEGVVDHAGIAAALKKIGFDGVCTIEE
FRPEYYEMSHDENVKKSAEVTRDFVAKYFA
Lactate dehydrogenase - AR1Y2_2463
Nucleotide sequence
(SEQ ID NO: 41)
atgacatataaaatgagaaaaatagtgatcataggggccggacatgtgggaaccatgtgcggcctgagtcttatgtaccgcgg
agaagtggacgaattggtattcatagacatcgataaggaaaaggcatacagccaggcgctggaccttagtgatgccgtctcct
tcctgccccggcagatcacggtgcgcaccggggattatgaagatgtggaggacgctgacatcatcgttatggccgctggtgttt
ccaggctgcccggtcagacaaggctggatatgctggacgattccatccgcatcatgaaagaagtcaccagccatttaaaaga
taagaagattcagggcatcctcatcagtatctccaatcctgccgacattgtggcggattatctgagaaaacaactgggccttccg
agaaaccgctgcttcagcaccggtacttccctggactcctgccgtctcagaaggattctttcccaaagaatgcacttagaccgg
aattccattcaggcctttgtcatgggagaacacggagattcccaaatgatcccgttttctgcggtacatatagccgggaaaccatt
gcttcagtggatgcaagatgaaccggaacggtacggaagtctcgatctttcacagattgaaacggagaccgccaacgccgg
ccatgaagtgatcgaaggaaagggcagcacagagtttggtatcggcatcgccctgtccgagatcgtccagtccattttccacg
attcaaaaaaggtccttcccgcttcgcctctgctggaaggagaatatggacagcatggcgttcacgccggtgtcccctgcgtgat
tggaaaagccggaattgaaaagatcattgagatccctctcacagaccgggagcaagagaaatttgaccattcctgcgatgtg
atcagaacgtttattcaagatctctga
Amino acid sequence
(SEQ ID NO: 42)
MTYKMRKIVIIGAGHVGTMCGLSLMYRGEVDELVFIDIDKEKAYSQALDLSDAVSFLPRQIT
VRTGDYEDVEDADIIVMAAGVSRLPGQTRLDMLDDSIRIMKEVTSHLKDKKIQGILISISNPA
DIVADYLRKQLGLPRNRCFSTGTSLDSCRLRRILSQRMHLDRNSIQAFVMGEHGDSQMIP
FSAVHIAGKPLLQWMQDEPERYGSLDLSQIETETANAGHEVIEGKGSTEFGIGIALSEIVQSI
FHDSKKVLPASPLLEGEYGQHGVHAGVPCVIGKAGIEKIIEIPLTDREQEKFDHSCDVIRTFI
QDL
Acetaldehyde dehydrogenase - AR1Y2_1414
Nucleotide sequence
(SEQ ID NO: 43)
atgggaagatttacattacctagagatttgtactttggaaaaggatcacttgagacactgaagacgctggacggaaaaaaagc
tgttttagtcatcggcggcggatctatgaaacgttttggatttgtagaccaggttgttgattatttaaaagaagcaggcatagagac
aaaattaattgagggagtagagccggacccttctgttgagaccgtatttaaaggtgccgagattatgagagagtttgaaccgga
ctggatcgtggcaatgggcggaggatctccgatcgatgctgcaaaagctatgtggatcttctatgaacatccggaaaccacattt
gacgatgtaaaagacccattcacagttccaaaactcagaaacaaagcaagattccttgcaatcccttcaacttcaggaacagc
tacggaagtgacggcatttgctgttatcacggactacagttcaggcattaaatatccgctggcagactttgagatcacaccggat
gttgccattgtagaccctgcactggcagagacgatgcctccgaaactgacctcttatacaggaatggatgcactgacccatgcg
attgaagcatatgttgccggactgcattcaccattttcagatcctttggcaatcgatgccatcgagatggtatttgacaacttaaaag
cttcctatgagggagacatgaccgcacgtgaaaatatgcactatgcacaatgtcttgcaggaatgtcattctctaatgcactgtta
ggaattgtacattctttagcgcataagacaggagcagtttttgatacaggacatatcacacacggatgtgcgaacgctatctatct
tccgtatgtgatccagtttaatgcaaaggtatgcgaagaccgctatgcagccattgcaagaaagatcggcctccctggaaattc
cgataaagcactgacactttctttgattgaagcaatccgggatatgaacaagaagctgaacattgtttccaacctgaaagatttc
ggaatcaatgaagaagaattcaaccagaagaagaggatgatcgcggagcgtgctgtggcagacgcatgtacaggatctaat
cctagagaaacctctgtagatgacatggaaaaagtgttagagtgcatctactacggaaaagacgtaatattctaa
Amino acid sequence
(SEQ ID NO: 44)
MGRFTLPRDLYFGKGSLETLKTLDGKKAVLVIGGGSMKRFGFVDQVVDYLKEAGIETKLIE
GVEPDPSVETVFKGAEIMREFEPDWIVAMGGGSPIDAAKAMWIFYEHPETTFDDVKDPFT
VPKLRNKARFLAIPSTSGTATEVTAFAVITDYSSGIKYPLADFEITPDVAIVDPALAETMPPKL
TSYTGMDALTHAIEAYVAGLHSPFSDPLAIDAEMVFDNLKASYEGDMTARENMHYAQCLA
GMSFSNALLGIVHSLAHKTGAVFDTGHITHGCANAIYLPYVIQFNAKVCEDRYAAIARKIGLP
GNSDKALTLSLIEAIRDMNKKLNIVSNLKDFGINEEEFNQKKRMIAERAVADACTGSNPRET
SVDDMEKVLECIYYGKDVIF
Alcohol dehydrogenase - AR1Y2_2121
Nucleotide sequence
(SEQ ID NO: 45)
atgaaaataagtccgaaagtccgtgaatttttgtgccttaccgcacatcctcagggctgcagaaaaaatgttgaagatcagatta
catatgtaaaaaaacacagcggtgttgagaatgggccgaaaaaggtcctggtcatcggctcatccacgggatatggcctggc
atcaaggatcaccgctgcattcgggtgcggagcggataccttgggtgtcatgtttgagagaccggcggccggtaaacgtaccg
ccactgccggatggtataatacagctgcatttgaacaggcggcggccagagaaggcttatatgcgaagacggtcaacgcag
atgcattttccagggaagtaaaagaccaggtcatccaactgatcaagaaggatttcggtaaggttgacgctgtcatttacagtct
ggctgcaccgagaagaacgacagaggacggcacggtttatcagtctgtcttaaagacaacaggggaagattttacaaataa
gaacctgaaccttaaagataataccatcagtgagaaaaccatcgaggctgccacagaggaagaagtgaaagctaccgtga
aggtcatgggcggggaggactggaaggcatggatccaggcgcttaccgatgcggatgccattgaaaagggcgcagtcacct
tcgcatattcttatatcggacctaagcttacatatcctatctattttgagggaaccatcggtgccgcgaagaagcatcttcataaga
cggcgggtgagatcaatgaggaattctcaaaggatggaatcaaagcatacatctccgtcaataaggggctggtgacacagg
ccagttctgccattccgatcgtaccgctttatatggctgtgctgtaccgcgttatgaaagaaaagggacttcatgaaggctgtatcg
aacagatcgtaaggctgttttctgaaaaggctgtctttggacaggccaagacagatgaacagggtctgatccgtctggatgact
gggaactgcgcccggaagtccaggaagaagtggaacgccgctgggagaaggtctgcaccgaaaatgtccaggagtattct
gatattgacggatactgggatgatttctaccaaatgttcggcttccgtatggatggagtggactacgaggaggatcttgacccttct
gtggatatcccaagcatttccggccaagaatag
Amino acid sequence
(SEQ ID NO: 46)
MKISPKVREFLCLTAHPQGCRKNVEDQITYVKKHSGVENGPKKVLVIGSSTGYGLASRITA
AFGCGADTLGVMFERPAAGKRTATAGWYNTAAFEQAAAREGLYAKTVNADAFSREVKD
QVIQLIKKDFGKVDAVIYSLAAPRRTTEDGTVYQSVLKTTGEDFTNKNLNLKDNTISEKTIEA
ATEEEVKATVKVMGGEDWKAWIQALTDADAIEKGAVTFAYSYIGPKLTYPIYFEGTIGAAKK
HLHKTAGEINEEFSKDGIKAYISVNKGLVTQASSAIPIVPLYMAVLYRVMKEKGLHEGCIEQI
VRLFSEKAVFGQAKTDEQGLIRLDDWELRPEVQEEVERRWEKVCTENVQEYSDIDGYWD
DFYQMFGFRMDGVDYEEDLDPSVDIPSISGQE
Pyruvate kinase - AR1Y2_2344
Nucleotide sequence
(SEQ ID NO: 47)
atgctgacaaaaaaaacaaagattgtttgcacgatggggcctgcgacagatgatgatgcggtgctcaaacagctgatgctgg
aaggaatggatatcgcgaggctgaatttctctcacggtgaccatgaggaacagcttggaaggatcaagagaattaaaagattc
cgtgaagaactgaatattccgatcgcgatcttactggatacaaaaggacctgagatccgtacaggacttctggagacagacgg
agacgtggaattaaaagaaggaaatgaatttgttctgaccacaagggacatcaaaggaaatgattctatcgtagcaattacat
acgaagagcttccgcaggatgtcacagaaggaaatacaatcctgatcgatgacggtctgatcgaactgagagtcaaagaga
tcaaagacggaacagatattgtatgtgaaattgtaaacggcggacttttaggaagccgtaagggagtgaacgtaccgaacgt
gcgcgtaaatcttccttccatcacagaaaaggataagagagacattgagttcggacttgaaaacggcattgattttatcgcggct
tctttcatccgtaacgcagatgcagtcaatgagatcaaggacatcgtaaaagcccacaacatggaagtcggcgttatctccaa
aattgaaaacgtggagggtgtggaaaatattgaatccatcatccaggcttctgacggaatcatggtagcccgtggagatttaggt
gtggaaattcccgcagaggaagttccgtttatgcagaaagcaatcattaagaaatgcaacaatgcatttaagccagtcatcac
agctacacagatgttagactctatgatcagaaacccacgtccgacaagagcggaagtaacagacgttgccaatgctatttatg
acggaaccgatgcgattatgctttctggagaaaccgcgaaggggaaataccctgtggaggccgtaaggatgatgaaccaga
tcgccatctccacagaactgcatctggatacgaagatcaaagattttcgttccatctatgtaagccgtggaatctcagcagcggtt
gcttattctgctgtacagactgctcataatattaacgcaaaatgtatcctggcgtccagtatgtccggatttacggcaaggatcgtat
ctaagttcaaaccggatgcacagatcattggcttgtctccggatgaagcggttgtgcgtaagatgcagctttactggggagtgcg
tccgtttaagtcacataaggcttcctctaccagagaacttttggatgaagctacggatatcgtactgaatgcagatcttgcacagg
agaacgacatcctggtactgaccggaggaggtccggcagaccaccgtggaaaaggcgtgacaaacatgctgaaagtagta
aaaatcggcgaataa
Amino acid sequence
(SEQ ID NO: 48)
MLTKKTKIVCTMGPATDDDAVLKQLMLEGMDIARLNFSHGDHEEQLGRIKRIKRFREELNIP
IAILLDTKGPEIRTGLLETDGDVELKEGNEFVLTTRDIKGNDSIVAITYEELPQDVTEGNTILID
DGLIELRVKEIKDGTDIVCEIVNGGLLGSRKGVNVPNVRVNLPSITEKDKRDIEFGLENGIDFI
AASFIRNADAVNEIKDIVKAHNMEVGVISKIENVEGVENIESIIQASDGIMVARGDLGVEIPAE
EVPFMQKAIIKKCNNAFKPVITATQMLDSMIRNPRPTRAEVTDVANAIYDGTDAIMLSGETA
KGKYPVEAVRMMNQIAISTELHLDTKIKDFRSIYVSRGISAAVAYSAVQTAHNINAKCILASS
MSGFTARIVSKFKPDAQIIGLSPDEAVVRKMQLYWGVRPFKSHKASSTRELLDEATDIVLN
ADLAQENDILVLTGGGPADHRGKGVTNMLKVVKIGE

Should there be an inconsistency between the sequences disclosed in the description and the sequences disclosed in the sequence listing, the sequences disclosed in the description are preferred. Alternatively, the sequences of the sequence listing may be used.

Experimental Support

Bacterium Comprising Myo-Inositol to Propionate Pathway

The present inventors found that myo-inositol can serve as carbon and energy source for a bacterium according to the present disclosure, e.g. Anaerostipes rhamnosivorans as previously isolated from stool of a healthy infant (Bui et al 2014 International Journal of Systematic and Evolutionary Microbiology 64: 787-793).

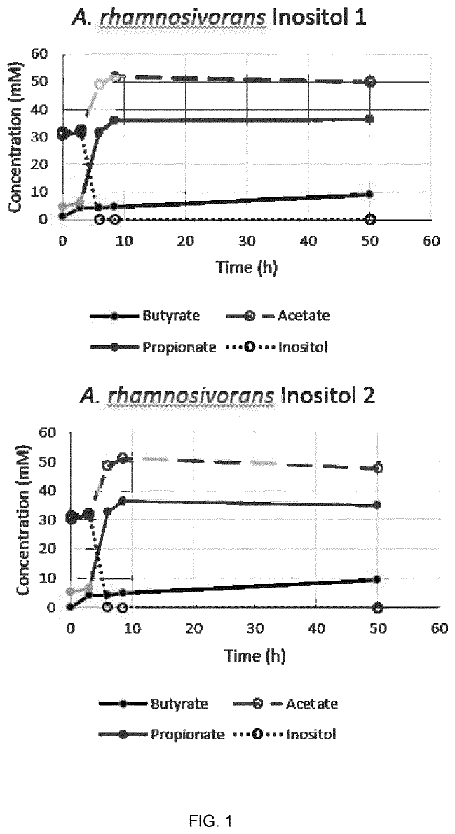

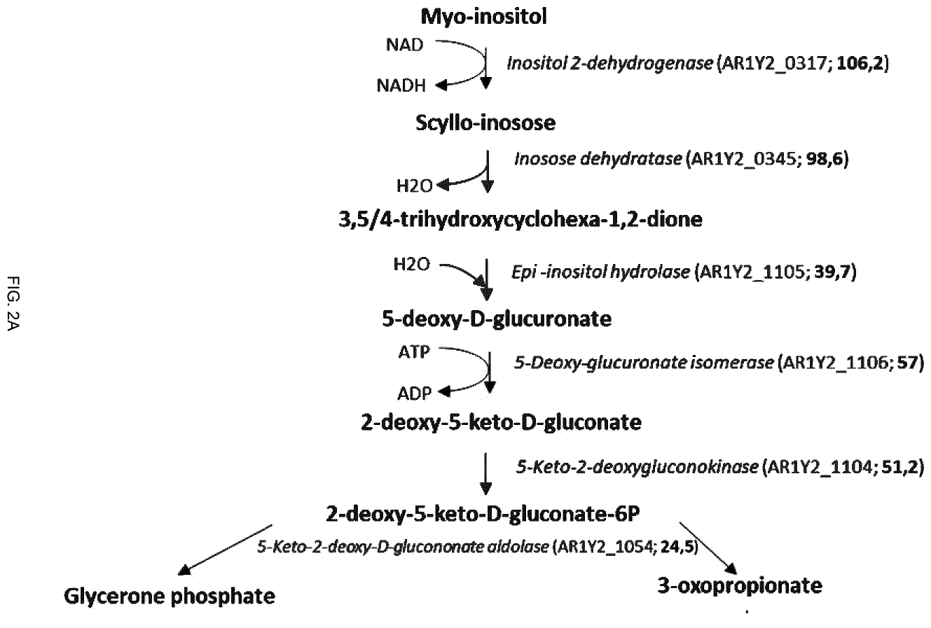

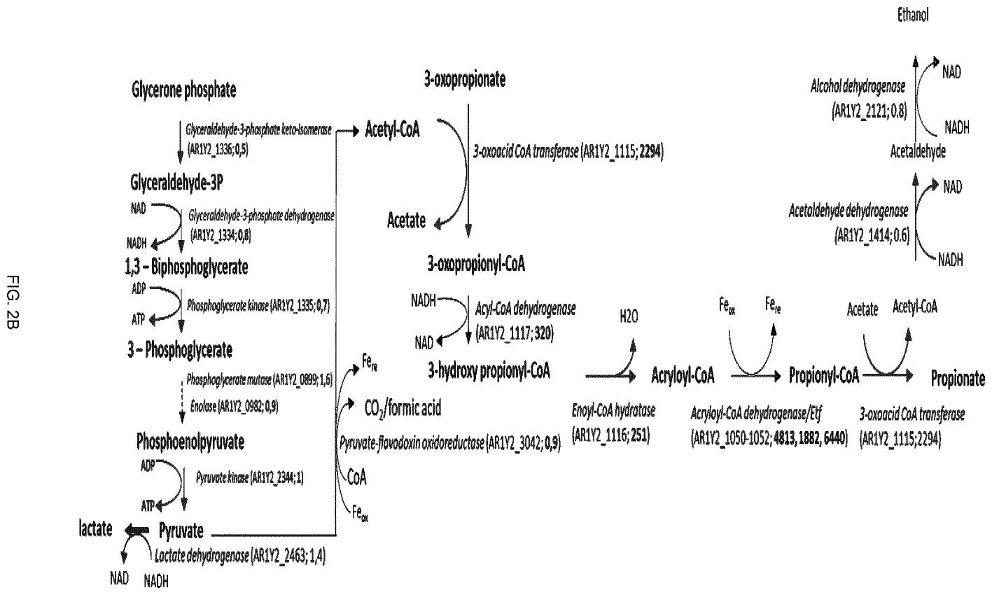

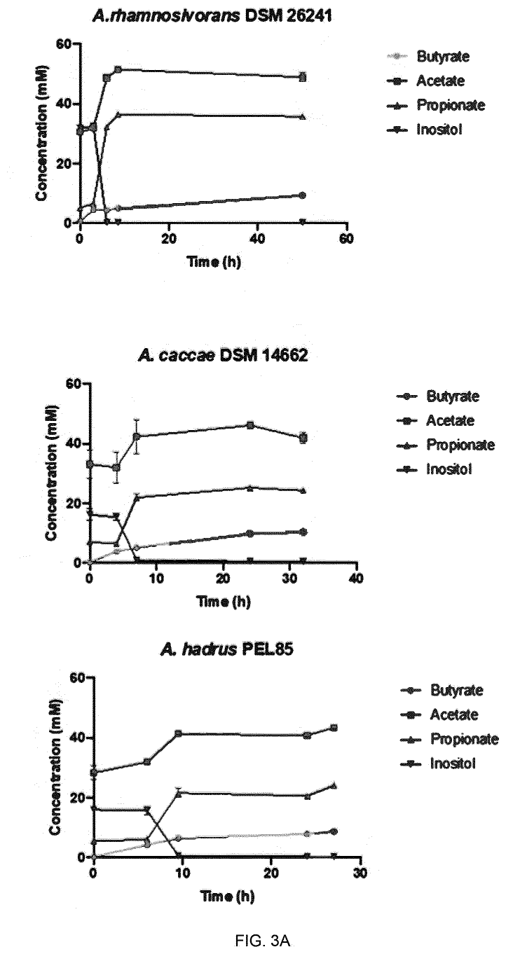

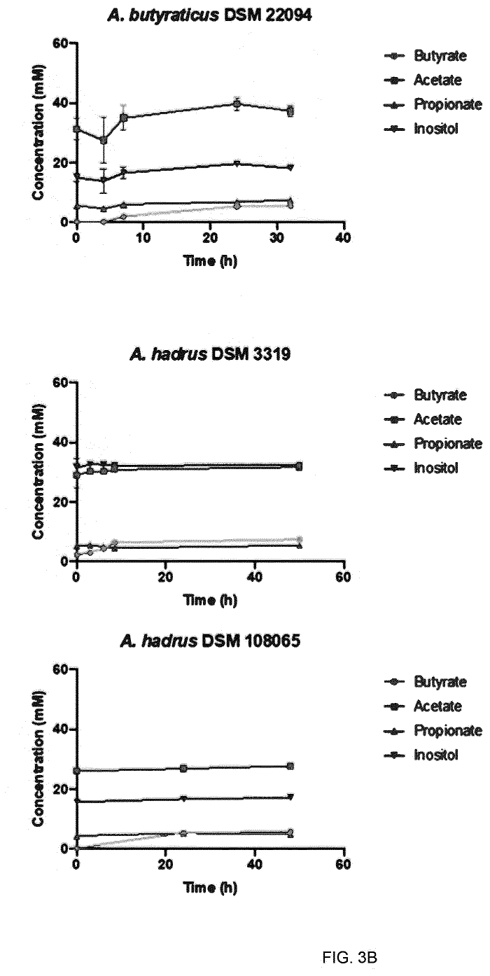

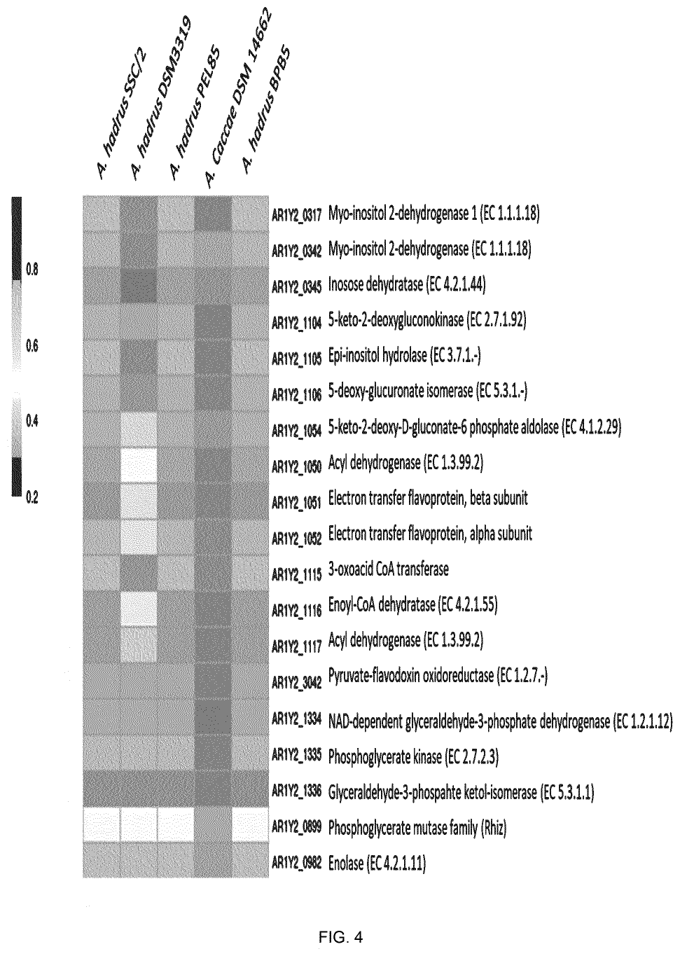

FIG. 1 shows that this bacterium is able to ferment efficiently myo-inositol to propionate. The experiment was performed twice.

Proteomics

A complete inositol fermentation pathway has been identified in the closed genome of Anaerostipes rhamnosivorans, which can thus be introduced in any bacterium of choice.

Method for Proteomics

To identify the proteins involved in the pathway, the protein abundances in cultures growing with two different substrates were examined with liquid chromatography-mass spectrometry/mass spectrometry.

Anaerostipes rhamnosivorans was grown in 4 replicates in 100 ml bicarbonate-buffered medium containing 40 mM myo-inositol or containing 40 mM rhamnose as carbon and energy sources.

Cells were collected in the exponential phase by centrifugation at 4700 g at 4° C. for 20 min. Cell pellets were washed twice and suspended in 0.5 ml of 100 mM Tris-HCl (pH 8). Protein extraction and digestion were performed as described previously (Bui et al., 2015). After the digestion, the samples were kept at −20° C. upon the analysis. The samples were analysed by injecting 4 μl sample over 0.10*250 mm ReproSil-Pur 120 C18-AQ 1.9 μm beads analytical column (prepared in-house) with an acetonitrile gradient at a flow of 0.5 ul/min with a Thermo EASY nanoLC1000. The gradient consisted of an increase from 9 to 34% acetonitrile in water with 1 ml/l formic acid in 50 minutes followed by a fast increase in the percentage acetonitrile to 80% (with 20% water and 1 ml/l formic acid in both the acetonitrile and the water) in 3 minutes as a column cleaning step. An electrospray potential of 3.5 kV was applied directly to the eluent via a stainless steel needle fitted into the waste line of the micro cross. Full scan positive mode FTMS spectra were measured between m/z 380 and 1400 on a Q-Exactive HFX (Thermo electron, San Jose, Calif., USA) in the Orbitrap at high resolution (60000). MS and MSMS AGC targets were set to 3.10⁶, 50000 respectively or maximum ion injection times of 50 ms (MS) and 25 ms (MSMS) were used. HCD fragmented (Isolation width 1.2 m/z, 24% normalized collision energy) MSMS scans of the 25 most abundant 2-5+ charged peaks in the MS scan were recorded in data dependent mode (Threshold 1.2e5, 15 s exclusion duration for the selected m/z+/−10 ppm). LCMS runs with all MSMS spectra obtained were analysed with MaxQuant 1.6.3.4 (Cox et al., 2008) using the “Specific Trypsin/P” Digestion mode with maximally 2 missed cleavages and further default settings for the Andromeda search engine (First search 20 ppm peptide tolerance, main search 4.5 ppm tolerance, MSMS fragment match tolerance of 0.5 Da, Propionamide (C) set as a fixed modification, while variable modifications were set for Protein N-terminal Acetylation and M oxidation which were completed by non-default settings for de-amidation of N and Q, the maximum number of modifications per peptide was 5 (Cox et al., 2011). Anaerostipes rhamnosivorans database from NCO (CP040058) was used together with a contaminants database that contains sequences of common contaminants like Trypsins (P00760, bovin and P00761, porcin) and human keratins (Keratin K22E (P35908), Keratin K1C9 (P35527), Keratin K2C1 (P04264) and Keratin K1CI (P35527)). The “label-free quantification” as well as the “match between runs” options were enabled. De-amidated peptides were allowed to be used for protein quantification and all other quantification settings were kept default.

Filtering and further bioinformatic analysis of the MaxQuant/Andromeda workflow output and the analysis of the abundances of the identified proteins were performed with the Perseus 1.6.2.1 module (available at the MaxQuant suite). Accepted were peptides and proteins with a false discovery rate (FDR) of less than 1% and proteins with at least 2 identified peptides of which at least one should be unique and at least one should be unmodified. Reversed hits were deleted from the MaxQuant result table as well as all results showing a normalised label free quantitation intensity (LFQ) value of 0 for all inositol and rhamnose injections. The normal logarithm was taken from protein LFQ MS1 intensities as obtained from MaxQuant. Zero “Log LFQ” values were replaced by a value of 5.9 (A value slightly lower than the lowest measured value) to make sensible ratio calculations possible. Relative protein quantitation of inositol to rhamnose was done with Perseus by applying a two sample Ttest using the “LFQ intensity” columns obtained with FDR set to 0.05 and S0 set to 1. nLC-MSMS system quality was checked with PTXQC (Bielow et al., 2016) using the MaxQuant result files.

### REFERENCES

Bui T P N, Ritari J, Boeren S, de Waard P, Plugge C M, de Vos W M. (2015). Production of butyrate from lysine and the Amadori product fructoselysine by a human gut commensal. Nature Communication 6.
Cox, J.; Mann, M. (2008). MaxQuant enables high peptide identification rates, individualized p.p.b.-range mass accuracies and proteome-wide protein quantification. Nature Biotechnology 26(12):1367-1372.
Cox, N. Neuhauser, A. Michalski, R. A. Scheltema, J. V. Olsen and M. Mann (2011). Andromeda: A Peptide Search Engine Integrated into the MaxQuant. Environment. Journal of Proteome Research. 10(4): 1794-1805.
Bielow, C., G. Mastrobuoni and S. Kempa (2016). Proteomics Quality Control: Quality Control Software for MaxQuant Results. Journal of Proteome Research 15(3): 777-787.

Beneficial Effect

It is known that dietary myo-inositol supplementation can reduce insulin resistance. The mechanism through which this occurs, however, is unknown. The present inventors considered that myo-inositol may have an indirect beneficial effect.

The present inventors envisaged that it is rather the metabolic conversion of myo-inositol that is beneficial to the host, rather than the myo-inositol itself. Hence, it is of interest to note that the production of butyrate and, notably, propionate, by A. rhamnosivorans is certainly beneficial for the patient (Hamer et al 2008 Pharmacology & Therapeutics 27: 104-119; Hosseini et al 2011 Nutrition Reviews 69: 245-258).

Hence, the supplementation of the patient with a bacterium according to the present disclosure, e.g. A. rhamnisovorans or alternatively both myo-inositol and A. rhamnosivorans provides a direct or synergistic effect on improved insulin resistance via additional propionate and butyrate production.

Treatment

Patients are treated as indicated below, according to the following 3 treatment arms: Treatment 1: 6 weeks, twice daily empty supplement.

Treatment 2: 6 weeks, twice daily supplement with 10⁸ living cells of a bacterium according to the present disclosure, i.e. A. rhamnisovorans.

Treatment 3: 6 weeks, twice daily supplement with 10⁸ living cells of a bacterium according to the present disclosure, i.e. A. rhamnisovorans in combination with 10 mg myo-inositol.

| | | Effect | Effect | Effect |
|---|---|---|---|---|
| No. of | | treatment 1 | treatment 2 | treatment 3 |
| patients | Condition | (patient 1) | (patient 2) | (patient 3) |
| 3 | Insulin | No effect | Reduction | Reduction of |
| | resistance | | of Insulin | Insulin |
| | | | resistance | resistance with |
| | | | with 10% | 12.5% |

It is expected that results similar to the putative effects as shown in the table above can be obtained with larger patient cohorts.

Other Host Bacteria

Another host bacterium, i.e. Escherichia coli is transfected according to standard methods with a gene set according to the present invention and comprising myo-inositol 2-dehydrogenase, inosose dehydratase, epi-inositol hydrolase, 5-deoxy-glucuronate isomerase, 5-keto-2-deoxygluconokinase, 5-keto-2-deoxy-D-gluconate-6 phosphate aldolase, glyceraldehyde-3-phosphate ketol-isomerase, glyceraldehyde-3-phosphate dehydrogenase, phosphoglycerate kinase, phosphoglycerate mutase, enolase, pyruvate kinase, pyruvate-flavodoxin oxidoreductase, 3-oxoacid CoA transferase. acyl-CoA dehydrogenase, enoyl-CoA hydratase, acryloyl-CoA dehydrogenase/Etf, and inosose isomerase. It is found that the host bacterium is capable of converting (myo)inositol to propionate under anaerobic conditions.

1. Bacterium comprising a gene set that allows said bacterium to convert inositol to propionic acid or a salt or ester thereof, for use in preventing and/or treating metabolic syndrome, insulin resistance or insulin resistance-related condition, wherein the gene set comprises genes encoding one or more of the proteins: myo-inositol 2-dehydrogenase, inosose dehydratase, epi-inositol hydrolase, 5-deoxy-glucuronate isomerase, 5-keto-2-deoxygluconokinase, 5-keto-2-deoxy-D-gluconate-6 phosphate aldolase, glyceraldehyde-3-phosphate ketol-isomerase, glyceraldehyde-3-phosphate dehydrogenase, phosphoglycerate kinase, phosphoglycerate mutase, enolase, pyruvate kinase, pyruvate-flavodoxin oxidoreductase, 3-oxoacid CoA transferase. acyl-CoA dehydrogenase, enoyl-CoA hydratase, acryloyl-CoA dehydrogenase/Etf, and inosose isomerase.

2. The bacterium for use according to claim 1, wherein at least one of the genes encoding any one or more of the proteins: myo-inositol 2-dehydrogenase, inosose dehydratase, epi-inositol hydrolase, 5-deoxy-glucuronate isomerase, 5-keto-2-deoxygluconokinase, 5-keto-2-deoxy-D-gluconate-6 phosphate aldolase, glyceraldehyde-3-phosphate keto-isomerase, glyceraldehyde-3-phosphate dehydrogenase, phosphoglycerate kinase, phosphoglycerate mutase, enolase, pyruvate kinase, pyruvate-flavodoxin oxidoreductase, 3-oxoacid CoA transferase. acyl-CoA dehydrogenase, enoyl-CoA hydratase, acryloyl-CoA dehydrogenase/Etf, and inosose isomerase, is overexpressed when the bacterium is grown in the presence of inositol as compared to when the bacterium is grown in the absence of inositol.

3. The bacterium for use according to claim 1, wherein the use is in preventing and/or treating insulin resistance or insulin resistance-related condition.

4. The bacterium for use according to claim 3, wherein the insulin resistance-related condition is chosen from dyslipidemia, type 2 diabetes mellitus and insulin-resistance in endocrine disease.

5. The bacterium for use according to claim 1, wherein the bacterium is a human intestinal isolate bacterium.

6. The bacterium for use according to claim 1, wherein the bacterium is deposited as CBS 140182.

7. The bacterium for use according to claim 1, wherein the bacterium is comprised in a composition further comprising a physiologically acceptable carrier.

8. The bacterium for use according to claim 7, wherein the composition is a food composition.

9. The bacterium for use according to claim 7, wherein the composition is a pharmaceutical composition.

10. The bacterium for use according to claim 7, wherein in the composition the bacterium is present in lyophilized or microencapsulated form.

11. The bacterium for use according to claim 1, wherein the bacterium is present in an amount within the range of between 10⁶ to 10¹⁵ colony forming units (CFU).

12. The bacterium for use according to claim 1, wherein the bacterium is administered separately, sequentially or simultaneously with inositol.

13. The bacterium for use according to claim 1 for use as a probiotic.

14. The bacterium for use according to claim 2, wherein the use is in preventing and/or treating insulin resistance or insulin resistance-related condition.

15. The bacterium for use according to claim 14, wherein the insulin resistance-related condition is chosen from dyslipidemia, type 2 diabetes mellitus and insulin-resistance in endocrine disease.

16. The bacterium for use according to claim 2, wherein the bacterium is a human intestinal isolate bacterium.

17. The bacterium for use according to claim 2, wherein the bacterium is deposited as CBS 140182.

18. The bacterium for use according to claim 2, wherein the bacterium is comprised in a composition further comprising a physiologically acceptable carrier.

19. The bacterium for use according to claim 8, wherein the food composition is a dairy product.

20. The bacterium for use according to claim 9, wherein the composition is a pharmaceutical composition in solid dosage form.


SEQUENCE LISTING (48 sequences)

SEQ ID NO: 1 - DNA, 1014 bp, Anaerostipes rhamnosivorans
atggtaaatgtaggtattatcggagcaggaagaatcggaagagtacatgtagaaagcatctgcacacaggtaggaaacgcaaaggttaagactcttgctgatccttttatgtcagaggacacagcagtatgggcaaaagaaatgggtgttgaaaataccaccaaagattacaaagacatcttaaatgatcctcagatcgacgcagtgctgatctgctcatccacagatacacattcaccgatctccgtagaggctattgaagcaggaaaacatgtattctgcgaaaagccaatcgaccatgacgtacagaagattcaggaagtagtcgacgctttaaagaaaagcgacgtaaaataccaggtaggattcaaccgccgttttgaccataactttgaagccatccgggatgccgtggctgctggaaagattggtgagacacagatcatcaaagttacatccagagatcctgaaccgccaagtgctgattatgtaaaagtttccggaggaatgttcctggacatgacaatccatgattttgacatggtccgtttccttgcaggatgtgacgctgaagaagtgtatgtagaagctgcagtccttgtagatcctgccatcggtgaagccggagatgtggacactgctgttatcacattaaaaatgaaaaacggttccatcgccgtgattgacaactcaagaaaagccgtttacggatacgatcagagagctgaagtgttcggatcaaagggaatggtcgtaacagcaaacgattccgcatcctcagccgtgatcagtaatgcggacggagtgacaggagaaaaacctctttacttcttcctggaacgctacatggcagcctatgcaaaagaaatcaaatgctttatcgacgccatcgaaaaaaacacagacacccctcttggagttatggacggtcttgagccagtacttatggggcttgcggctaaaaaatcattagaagaacaccgcccagtaaaaatttctgaaattacagtatag

SEQ ID NO: 2 - protein, 337 aa, Anaerostipes rhamnosivorans
MVNVGIIGAGRIGRVHVESICTQVGNAKVKTLADPFMSEDTAVWAKEMGVENTTKDYKDILNDPQIDAVLICSSTDTHSPISVEAIEAGKHVFCEKPIDHDVQKIQEVVDALKKSDVKYQVGFNRRFDHNFEAIRDAVAAGKIGETQIIKVTSRDPEPPSADYVKVSGGMFLDMTIHDFDMVRFLAGCDAEEVYVEAAVLVDPAIGEAGDVDTAVITLKMKNGSIAVIDNSRKAVYGYDQRAEVFGSKGMVVTANDSASSAVISNADGVTGEKPLYFFLERYMAAYAKEIKCFIDAIEKNTDTPLGVMDGLEPVLMGLAAKKSLEEHRPVKISEITV

SEQ ID NO: 3 - DNA, 1020 bp, Anaerostipes rhamnosivorans
atgaacaaagtaagagtaggtattgctggtttaggaagattagggaaattacatgcagagaatatcgcattcaagattccaaacgcggaacttacagcggcctgttcgatcgcgccagaggaattggaatatgccacagaacatttcggggtaaccgatgtttatacagatttttatgagatgttagataaagcagacattgatgtcgtggcggtggtgaccacgagcggagaacactgctggcagatcaaagcagcattggacgctggaaaacatgttttttgcgataaacctctcggggttacggtcaatgaatgtaaaacagcggaggccgcggtggaacgccacccagagcttacattttttcttggatttatgcgccgttatgacccatcctacgcttatgcgaaaaagaaaatccaggagggagccattggtaagccgtatatggtaaaagcggcagggatcgacccggaagctctggtggagggagcagtaaaatttgcagccaccagcggcggtatttttatcgacatggcaatccatgatatcgatttaatgcgctggcttttggaatccgatccggtggaagtctacgctgcaggggccacgtttaaacatcccgaattcagggaggcaggggatgacgagacgggggcagccatgtacaaatgtgagaacggtgcactgggatttgtacatgttgggcgtacagcggcccatggatatcatgtggagacagagatcatcggaacagagggaagcctgagaatcagtcctgtaccggaaaagaacctgtgtatgatctacgatgagaacggagctgtgacagaatgtgtcagcggttttgcagaacggtttgcagaggcatacctgttggaaatgaaggaattcatcaactgtgtccagagcggcacaaagccggatgtgaccgtatatgacggcacaaaatccacacagatcggttttgccacaacagaagcgtggaaaaagggctgtactgcaaagatccagtactag

SEQ ID NO: 4 - protein, 339 aa, Anaerostipes rhamnosivorans
MNKVRVGIAGLGRLGKLHAENIAFKIPNAELTAACSIAPEELEYATEHFGVTDVYTDFYEMLDKADIDVVAVVTTSGEHCWQIKAALDAGKHVFCDKPLGVTVNECKTAEAAVERHPELTFFLGFMRRYDPSYAYAKKKIQEGAIGKPYMVKAAGIDPEALVEGAVKFAATSGGIFIDMAIHDIDLMRWLLESDPVEVYAAGATFKHPEFREAGDDETGAAMYKCENGALGFVHVGRTAAHGYHVETEIIGTEGSLRISPVPEKNLCMIYDENGAVTECVSGFAERFAEAYLLEMKEFINCVQSGTKPDVTVYDGTKSTQIGFATTEAWKKGCTAKIQY

SEQ ID NO: 5 - DNA, 900 bp, Anaerostipes rhamnosivorans
atgtttaacaaagaaaaagtaaaattaggaattgccccgatcgcttggaccaacgatgatcttccggatcttggaaaagaaaacacctttgagcagtgtgtcagtgaaatggcattggcagggtacactggttccgaagtaggaaataaatatccaaaggacccagaggttctaaaaaaggccctgaaactccggggaatggagatctgcaatcagtggttctcatcatttttgatcacaaagccatttgaagaggtggaaaaagagttccgcgctcaactgagtttcttaaaggctgtgggagcaaagatcattggtgcttctgaacagagccacagtgtacagggtatgctggatactccgatctttggacataagtatgtgatgaatgacgaagagtgggaaaccttctgtacgggaatgaataagttaggaaaaatcgcaaaagaagaatacggcatcagcttaaccttccaccatcatatgggaactgtggtacaggacccggatgaagtggaaagaatgatggcaaacacagatcctgaatatgtgaatctgttatttgatacagggcattttacatactgcggggcagatcctcttgaaatggtgaaaaaatatgtggaccgcatcaaacatgtccatcttaaggatatccgtccggaaatcgtagaaaaagtgaaagcagaaaacttaagcttcttagatggtgtgcgtatgggggcgtttacggtaccgggtgacggatgtattgattttgatccgatctttaaggtgcttgaagattcaggatatgaaggatatatgctggtagaggcagaacaggatccggccaaggcaaatccgcttgaatatgccatgaaggcaagaaaattcatcgctgagaaaactggattataa

SEQ ID NO: 6 - protein, 299 aa, Anaerostipes rhamnosivorans
MFNKEKVKLGIAPIAWTNDDLPDLGKENTFEQCVSEMALAGYTGSEVGNKYPKDPEVLKKALKLRGMEICNQWFSSFLITKPFEEVEKEFRAQLSFLKAVGAKIIGASEQSHSVQGMLDTPIFGHKYVMNDEEWETFCTGMNKLGKIAKEEYGISLTFHHHMGTVVQDPDEVERMMANTDPEYVNLLFDTGHFTYCGADPLEMVKKYVDRIKHVHLKDIRPEIVEKVKAENLSFLDGVRMGAFTVPGDGCIDFDPIFKVLEDSGYEGYMLVEAEQDPAKANPLEYAMKARKFIAEKTGL

SEQ ID NO: 7 - DNA, 1932 bp, Anaerostipes rhamnosivorans
atgagtacaaccagaatgacagtagcacaggctcttgtaaaattccttgacaatcagtatgtggaatttgacggggaagaaacaaaatttgtagaaggtttctttactatcttcggacacgggatcgccgttggtctcggagaggcattggacagcaatcctgggagcttaaaggttcttcagggaagaaatgagcagggaatgtgccacgtagcgacagcttacgcaaaacaaaatagaagaaggaagatcattccttgtgcatcttctgtaggacccggagcggcaaatatggtgacagcagcggctacagctacagtgaacaatattccgcttttggtagtgccttctgacacatttgccacaagacagccggacccggtgctccagcagttagaaaagagtgacagtctctctacaacaacaaacgacgcattcaaaccggtttgtaaatattgggataggatcatccgcccggaacagctgatgacagccatggtcaacgctatgcgtgttctcacagatccgtcagagacaggcgctgtgtgcattgcactctcccaggatgtagaaggagaatcttatgatttccctgactatctgtttgaaaaacgtgtacataggatcacaagaccttgcgcggtaaaagaagagatcgaggaccttgtaaaagtcattaaagaagcgaaaaatccgttagtgatcgtgggcggaggtgtgaagtattctgaggcaggagaagctgtagaaaagttctgcgaagactttaacatcccgttcggagagagccaggcaggaaaaagtgcctgcaagtcaagccacccttactgcttaggaggaatcggcgtcacaggaaccctggctgccaataccatcgctgatgaagctgatgtggtcattgcagtgggaaccagactttcagactttacaacaggatctaaaggacagttcaaaaatcctaatgtaaaaatggtgacgatcaacaacaataaatatcatgcatataaaatggatgcagtaaaagctgtcggagacgcgaaagtgacagtggaagctctttctgctagactgaaagaggaaggatacaagtctgcttacacaacagagatccaggacgcaaaggcagcctgggataaagaaatggaccgccttgcgaatattgagtatacaggcgatgactttgagccggagatcaaagcgagagatccgcgtacgattcctgaattcgtaaaacttacagggggacttatcactcagactgcagcattgtgcaaaatccgtgaggtcattgatgaagacgctaatattgtgacagcaggggggagtcttccaagctgtatgcagagaatctggaccacagacaaacgaggcggatatcacgcagaatacggatattcctgcatgggatatgaagtggcgggagcgctcggcgtaaagatggcagagcctgagcatgaatcctacgtggttgtgggggatgccagctttcagatgctccacagtgagattatgacagcgatgcaggaaaaacaaaagatcaacatccttgtatttgacaactgcggatttggctgtatcaacaaccttcagatgaatcatggagtgggaagtcttgcaacagagttcagataccgcgatgaaaacaatgagatcaagggtgatctgatcccggttgactatgcaaaagtcggagaaggatacggactcaagacgtacagtgtaagaacgatcaaagagctggaggaagcattgatcgatgctaagaaacaggacgttgccacattgatcgacttgaaggtcattccgaaaacaatgacagacggttacaaatcatggtggaacgtaggtattgcgacaacttctgtgaaagaaagcgtgcagaaagcatgtgaaggagtattggaaggccgcaaaaacgcaagacagtattag

SEQ ID NO: 8 - protein, 643 aa, Anaerostipes rhamnosivorans
MSTTRMTVAQALVKFLDNQYVEFDGEETKFVEGFFTIFGHGIAVGLGEALDSNPGSLKVLQGRNEQGMCHVATAYAKQNRRRKIIPCASSVGPGAANMVTAAATATVNNIPLLVVPSDTFATRQPDPVLQQLEKSDSLSTTTNDAFKPVCKYWDRIIRPEQLMTAMVNAMRVLTDPSETGAVCIALSQDVEGESYDFPDYLFEKRVHRITRPCAVKEEIEDLVKVIKEAKNPLVIVGGGVKYSEAGEAVEKFCEDFNIPFGESQAGKSACKSSHPYCLGGIGVTGTLAANTIADEADVVIAVGTRLSDFTTGSKGQFKNPNVKMVTINNNKYHAYKMDAVKAVGDAKVTVEALSARLKEEGYKSAYTTEIQDAKAAWDKEMDRLANIEYTGDDFEPEIKARDPRTIPEFVKLTGGLITQTAALCKIREVIDEDANIVTAGGSLPSCMQRIWTTDKRGGYHAEYGYSCMGYEVAGALGVKMAEPEHESYVVVGDASFQMLHSEIMTAMQEKQKINILVFDNCGFGCINNLQMNHGVGSLATEFRYRDENNEIKGDLIPVDYAKVGEGYGLKTYSVRTIKELEEALIDAKKQDVATLIDLKVIPKTMTDGYKSWWNVGIATTSVKESVQKACEGVLEGRKNARQY

SEQ ID NO: 9 - DNA, 786 bp, Anaerostipes rhamnosivorans
atgggtaaagtattcggctatcctgagtttgatgtaaacggggaaaaggttttaacaacatatgacaatgattataaagaaatgctgatggacatccgcgtcttccgcttaaaagcgggagatgtgaagaccttctgcaaagaaaaagaggagaccgcggttcttttactaaaaggggatgtcgtattctcatttgatgaaacttccaaagaagtcacaagaaaagatgtatttacagaaggaccttgggcagtacatatctgcacaggagccaaagtcatagtggaagcaaaggcagactctgaaatcttggtacagtccacaatgaatgaccagacatttgagagtaaattgtatgcgccggaagatgctccttggggatattcctgtgtaggaaagttcggaaatgtggctaaacgccgtgtcaataccatctttgaccatgacattgcaccatactccaacatggtattaggtgaagtcttaaatgaccgcggaaactggtcaggatatcttcctcacagacatccgcagccggaaacttattacttcctgtttgaccatccggaaggatttggggcaagctttgtgggagaccaggtatttaagagtacagacggaagcttctcagcgattccagggggagaacttcatcctcaggcagtggctccgggataccaaatgtatacttgctggatgatccgtcatctggaagggaacccatggcttcagacagacagaaatgaagacgaacgctacgtttggcttcatgacgcagaattctaa

SEQ ID NO: 10 - protein, 261 aa, Anaerostipes rhamnosivorans
MGKVFGYPEFDVNGEKVLTTYDNDYKEMLMDIRVFRLKAGDVKTFCKEKEETAVLLLKGDVVFSFDETSKEVTRKDVFTEGPWAVHICTGAKVIVEAKADSEILVQSTMNDQTFESKLYAPEDAPWGYSCVGKFGNVAKRRVNTIFDHDIAPYSNMVLGEVLNDRGNWSGYLPHRHPQPETYYFLFDHPEGFGASFVGDQVFKSTDGSFSAIPGGELHPQAVAPGYQMYTCWMIRHLEGNPWLQTDRNEDERYVWLHDAEF

SEQ ID NO: 11 - DNA, 1017 bp, Anaerostipes rhamnosivorans
atgaagtatgttgaatttgaccagacaagaccaatggatttagtactcatcggaagggtagccatcgattttaacccggtagattatttccacccgttatctgagtgcactaccttcaagaagtatgtgggaggatctccggcaaacatcgcagtgggcatcacaaggcacggaaagaaagccggattcatcggcaaagtttctgatgaccagttcggagagtatgtagtagacttcttcaataaagagggaatcgatacatcccatattacaaaatgtacgaacggagaaaagcttggacttacctttacagaaattctttccagagaagaaagcagcatcttaatgtaccgcaactgcatcgcagacttacagttatctgtggatgacattgacgaagaatacataaagaacacaaaagcaatcctggtttccggaacagcattggcacagagcccatccagggaagcagtgttaaaagcagtgatgcttgccaagaaaaatgatacaaagatcatctttgacattgactatagagaatacaactggaaaaattccgatgagatttcaatctactattctttagtagccagagaagcagatatcatcatgggatctagagaagaatttgacctgacagaaaaactgataaaaccgggcagaacagacaaagaatctgcacaggcatggaacactgaaaatgcaaagatcgttgtcatcaaacatggcaaaaaaggatctacagcatataccaatgacggaaatgactattcaatcaaaccgttcccggttgaggcgttaaaaggctttgggggcggagacggatatgcatccggcttcttatatggaatatttgaaggatgggatatgatcgagtgtctggaattcggaagtgcggaagcggccatgatggtgagaagccattcttgttctgaagaacttccaaaccctcaggaagtcaaagactttatccgcgagacaaaagacaaatatggcgaaatgattgcgagagtatag

SEQ ID NO: 12 - protein, 338 aa, Anaerostipes rhamnosivorans
MKYVEFDQTRPMDLVLIGRVAIDFNPVDYFHPLSECTTFKKYVGGSPANIAVGITRHGKKAGFIGKVSDDQFGEYVVDFFNKEGIDTSHITKCTNGEKLGLTFTEILSREESSILMYRNCIADLQLSVDDIDEEYIKNTKAILVSGTALAQSPSREAVLKAVMLAKKNDTKIIFDIDYREYNWKNSDEISIYYSLVAREADIIMGSREEFDLTEKLIKPGRTDKESAQAWNTENAKIVVIKHGKKGSTAYTNDGNDYSIKPFPVEALKGFGGGDGYASGFLYGIFEGWDMIECLEFGSAEAAMMVRSHSCSEELPNPQEVKDFIRETKDKYGEMIARV

SEQ ID NO: 13 - DNA, 837 bp, Anaerostipes rhamnosivorans
atggcattagtcaaaatgaaacagttattagagcaggcaaaaaaggaaaacagaggatgcggtgcattcagtgtgggcaatatggaaatggtcaaaggggcgattcaggcagcggaggagatgaatacaccgatcatcctgcagatcgctgaagtgcggctcgcccattcaccgcttcatcttatggggccgatgatggtgcaggcggcaaaagaggcaaaggtggatgtggcagttcatctggaccacggacttaacattgagactgtaaaaaaagcgctggaactgggatttacctctgttatgctggatgcatccagagattcgtttgaagaaaatatcagaaaaacaaaagaagttgtcgctctggcagcacagtacggagcaaccgtggaagcggaattagggctggtaggcggaagcgaagacggaaagagtgaccatggaatccagtgcaccaagcctcaggacgccaaggtcttctgtgagcagaccggagtggatgccctggccgttgccattggaaatgcccacggggactatccggtagctccgactttggaatttggtgtattggaagacattcacaatattactgccaaaccattggtgcttcacggcggaagcggtattacagatgaggatttcagaaaagccatctctcttggaatctgcaaagtgaacattgcaactgcaagctttaacagcctgacaaaagaagtggaaaactatctggagacagaaggaaaacataattacttcgatatgaacgaagctatggtaaagggtgtctatgacaacgtaaaacgccatatccagatctttaactgcagataa

SEQ ID NO: 14 - protein, 278 aa, Anaerostipes rhamnosivorans
MALVKMKQLLEQAKKENRGCGAFSVGNMEMVKGAIQAAEEMNTPIILQIAEVRLAHSPLHLMGPMMVQAAKEAKVDVAVHLDHGLNIETVKKALELGFTSVMLDASRDSFEENIRKTKEVVALAAQYGATVEAELGLVGGSEDGKSDHGIQCTKPQDAKVFCEQTGVDALAVAIGNAHGDYPVAPTLEFGVLEDIHNITAKPLVLHGGSGITDEDFRKAISLGICKVNIATASFNSLTKEVENYLETEGKHNYFDMNEAMVKGVYDNVKRHIQIFNCR

SEQ ID NO: 15 - DNA, 747 bp, Anaerostipes rhamnosivorans
atgcgtaaaacaatcgtagcaggaaactggaaaatgaacaaaacacctagcgagacagtagctttagttaatgaattaaagccattggtagcaagcgaggtttcagatgttgtcttctgtgtgccggccatctgtattccggcagcactggaagctgcaaaaggaagtaacatcaacatcggcgctgagaacatgtattttgaagagagcggcgcttacacaggcgagatcgctccgaatatgttaacagacttaggtgtgaaatatgttatcatcggacattctgagagaagagaatattttgcagaaacagatgagacagtgaataaaaaagtcttaaaagcttttgaacatggaattactccaatcgtttgctgcggtgagacacttgctcagagagagcagggtgtgaccatcgatttcgtacgccagcagatcaagatcgcattccagggtgtgactgctgaccaggcaaagacagctgtcattgcatacgaaccgatctgggctatcggaacaggaaaagttgcgacgacagaacaggctcaggaagtatgtaaagcgatccgtgagtgcatcgctgaagtatacgatcaggcaacagcggacgctattcgtatccagtacggcggttctgtatccgctgcaagtgcaccagaattatttgcacaggaagacatcgatggaggattagtcggtggagctgctttaaaaccagactttggtaagatcgtgaattacaaataa

SEQ ID NO: 16 - protein, 248 aa, Anaerostipes rhamnosivorans
MRKTIVAGNWKMNKTPSETVALVNELKPLVASEVSDVVFCVPAICIPAALEAAKGSNINIGAENMYFEESGAYTGEIAPNMLTDLGVKYVIIGHSERREYFAETDETVNKKVLKAFEHGITPIVCCGETLAQREQGVTIDFVRQQIKIAFQGVTADQAKTAVIAYEPIWAIGTGKVATTEQAQEVCKAIRECIAEVYDQATADAIRIQYGGSVSAASAPELFAQEDIDGGLVGGAALKPDFGKIVNYK

SEQ ID NO: 17 - DNA, 1017 bp, Anaerostipes rhamnosivorans
atggcagtaaaagtagcgattaatggatttggacgtattggacgtcttgcattcagacagatgttcggagcagaaggttatgaagttgtagcaatcaacgatttaacaaatcctaagatgttagcacatttattaaagtatgattctgcacagggaagatatgcattaggcgacaaggtagaagcaaaagaatcttccatcgtagttgacggaaaagaaatcgaaatctacgcagaagcagacgcaacaaaacttccttggggagaattagatgttgatgttgtattagaatgtacaggattctacacatctaaagacaaagcatccgcacacgttcaggcaggagctaagaaagttgttatttctgcgccagctggaaatgatcttccaactatcgtatataatgtaaaccacgatacattgacaaaagaagacacagtcatctcagcagcatcttgtacaacaaactgcttagcacctatggctgatgcactgaacaagtatgcaccaatccagtctggtatcatgttaacagtacatgcttacacaggagatcagatgactcttgacggacctcagagaaaaggtgacttaagaagagcacgtgcagctgcagtcaacatcgtacctaactctacaggtgctgcaaaagctatcggattagttatcccagaattaaacggaaaattaatcggatctgctcagcgtgtaccggttcctacaggatcaacaacacttttaacagcagttgttaaaggtgacaatgtaactgttgaaggaatcaacgcagctatgaaagcagcagcaacagagtctttcggatacacagaagaagaattagtatcttctgacatcatcggaatcaaatatggttctttattcgacgcaactcagacaatggttactgaagttggtgacggattatatgaagtacaggttgtttcttggtatgacaacgaaaactcatacacaagccagatggttagaacaatcaaatactttgcagaattagcataa

SEQ ID NO: 18 - protein, 338 aa, Anaerostipes rhamnosivorans
MAVKVAINGFGRIGRLAFRQMFGAEGYEVVAINDLTNPKMLAHLLKYDSAQGRYALGDKVEAKESSIVVDGKEIEIYAEADATKLPWGELDVDVVLECTGFYTSKDKASAHVQAGAKKVVISAPAGNDLPTIVYNVNHDTLTKEDTVISAASCTTNCLAPMADALNKYAPIQSGIMLTVHAYTGDQMTLDGPQRKGDLRRARAAAVNIVPNSTGAAKAIGLVIPELNGKLIGSAQRVPVPTGSTTLLTAVVKGDNVTVEGINAAMKAAATESFGYTEEELVSSDIIGIKYGSLFDATQTMVTEVGDGLYEVQVVSWYDNENSYTSQMVRTIKYFAELA

SEQ ID NO: 19 - DNA, 1194 bp, Anaerostipes rhamnosivorans
atgttaaacaagaaatcagtagatgatatcaacgtaaaaggaaaaaaagtcttagtgcgctgtgacttcaacgttccgcttcaggatggaaagatcacagatgagaaccgtcttgtggcagcacttcctacactaaagaagttaattgctgacggcggaaagctgatcctctgctcacacttaggaaaaccaaaaggggaaccgaaaccggagttatctcttgcgccagtagcagaaagattaagcgagttattaggacaggaagtaaagtttgctgcagatgacaacgtggtaggaccgaatgcaaaagctgcggtagaaggaatgcaggacggggatgtcattttacttcagaatacacgctacagagcagaagagacaaagaatggagaagagttcagcaaagaacttgcaagtctctgcgatgtttttgttaatgatgcttttggtaccgctcatagagctcactgctctaacgtgggagttacaaagttcgtggacactgcagttgtaggatatttaatgcagaaagagattgatttcttaggaaatgcggtcaacaatccggaacgtcctatggtcgctatcttgggaggagcaaaggtttcctctaagatctctgtgatcaataacctgctagacaaagtcgatactttgatcatcggcggaggtatggcatatactttcatgaaagcattgggagaagaagttggagattcccttctggaagctgactacttagactacgcaaaagaaatgatggacaaggcaaaagaaaaaggtgtgaacttactgattcctgttgacactgtagttgctgatgacttctcaaatgacgccaacatcaaaactgttgagagaggcggaatcgaagcaggatggcagggacttgacatcggaccaaagacaagagaactgttttcaaatgcactgaaagatgccaagactgtagtatggaacggaccgatgggatgctttgagatgccgaactttgcagcaggaaccgttgcagttgcagaagcactggcagagatcgacgctacaactatcattggcggaggagattccgcagcggcagttaaccagctcggattcggagataagatgactcatatctctacaggtggtggagcatcactggaattcctggaaggaaaagaattaccaggcgttgcagcggcaaacgataaataa

SEQ ID NO: 20 - protein, 397 aa, Anaerostipes rhamnosivorans
MLNKKSVDDINVKGKKVLVRCDFNVPLQDGKITDENRLVAALPTLKKLIADGGKLILCSHLGKPKGEPKPELSLAPVAERLSELLGQEVKFAADDNVVGPNAKAAVEGMQDGDVILLQNTRYRAEETKNGEEFSKELASLCDVFVNDAFGTAHRAHCSNVGVTKFVDTAVVGYLMQKEIDFLGNAVNNPERPMVAILGGAKVSSKISVINNLLDKVDTLIIGGGMAYTFMKALGEEVGDSLLEADYLDYAKEMMDKAKEKGVNLLIPVDTVVADDFSNDANIKTVERGGIEAGWQGLDIGPKTRELFSNALKDAKTVVWNGPMGCFEMPNFAAGTVAVAEALAEIDATTIIGGGDSAAAVNQLGFGDKMTHISTGGGASLEFLEGKELPGVAAANDK

SEQ ID NO: 21 - DNA, 606 bp, Anaerostipes rhamnosivorans
ttgaagttatatatgatgaggcacggagaaactccatggaataaagtgcggaggattcaggggcagtcagacgtggatctgagtgaagcgggcagggaagcagccaggctcaccaggagagcactgaaagatttccattttgatgcggcctacacgagcccgctgagcagagcgagagaaacaggggagatcatcctggagggcagagggatccctctgctggaggatgaacggcttaaggaggccaacttcggaccttatgaaggggcacacatcaatgaattatatgaaagagaagaaccgattctggatttctttgaccggccggaacagatggcctatattgaaggagcggagactccggctcaggtagtagaaagatccaggatatttttagaggaatttatacttccgatggaaaaagaagcagaaagtgtgatcttatttacccacggagctttcatccacgggatgctgactaatatgtatcaaagagagatcagggacttttggaaaggaccgaagcttaagaattgttctatcagtacggctgaagtcagggacggtcagttccgcattttgagtgaggctgaggtcttttactga

SEQ ID NO: 22 - protein, 201 aa, Anaerostipes rhamnosivorans
MKLYMMRHGETPWNKVRRIQGQSDVDLSEAGREAARLTRRALKDFHFDAAYTSPLSRARETGEIILEGRGIPLLEDERLKEANFGPYEGAHINELYEREEPILDFFDRPEQMAYIEGAETPAQVVERSRIFLEEFILPMEKEAESVILFTHGAFIHGMLTNMYQREIRDFWKGPKLKNCSISTAEVRDGQFRILSEAEVFY

SEQ ID NO: 23 - DNA, 1284 bp, Anaerostipes rhamnosivorans
atggattatttagagattgaaaaggtcataggaagggaaattattgattccaggggaaatcctacagtggaagcagaagttcttttagccggcgggacaatcggacggggcacagcacccagcggagcttcaactggagaatttgaggcgttggaattaagagacggagataaggagcgttttggaggaaaaggtgtgtccaaggcagttcataatattaatacgatcattaacgatactttaaaagggatggatgcatccgatacttacgcggtggacagagcgatgatcagcgtagacggaacaaaggataaatcaaagcttggggccaatgcgatccttgcggtttcgattgcatgcgcaaaggcggcatcggcaggcctggatctgccattataccgttttctgggcggtgttaatgccaaccgtcttccggtgccgatgatgaacattttaaacggaggtgcccatgcggccaacaccgtcgatgtacaggagtttatgatcatgcctgtgggagcagaaaatttcagggaagcactccggtggtgtacggaagtattccacgccttggcagctttgctgaaatccaaagggctggcaacctccgtgggtgatgaaggcgggtttgcgcctgaccttgcaagtgatgaggaagccatcgaatatattttggaagctgttcaggcagcgggatatgagccgggcagtgattttgttctggctatggatgctgcttccagcgagtggaaaggcgcgaacaaaggtgaatatattcttcctaagagtggcaagaaatttacctcagaagaactggtggaccactggaaatcactttgtgagaaatatccgatcagctccattgaggatggattggatgaggaagactgggacggctggaaatacatgacccaggagcttggagagaaaatccagctggtgggagatgacttatttgtaacgaatacggaacgccttggaaaaggtatcaagatgggatgcggaaattctatcttgatcaagctgaaccagatcggttctgtgtcggagacattggaagctattaagatggcccataaagcgggttacacagcggtcacatcccaccgttcaggggagacagaagacacgaccatcgcagatctcgctgttgcacttaataccggacagatcaaaaccggtgcaccgagcagaagtgagagggtggcaaaatataatcagcttctgaggattgaagaggaactgggggaaagtgccgtatacccgggagtcggaacattttag

SEQ ID NO: 24 - protein, 427 aa, Anaerostipes rhamnosivorans
MDYLEIEKVIGREIIDSRGNPTVEAEVLLAGGTIGRGTAPSGASTGEFEALELRDGDKERFGGKGVSKAVHNINTIINDTLKGMDASDTYAVDRAMISVDGTKDKSKLGANAILAVSIACAKAASAGLDLPLYRFLGGVNANRLPVPMMNILNGGAHAANTVDVQEFMIMPVGAENFREALRWCTEVFHALAALLKSKGLATSVGDEGGFAPDLASDEEAIEYILEAVQAAGYEPGSDFVLAMDAASSEWKGANKGEYILPKSGKKFTSEELVDHWKSLCEKYPISSIEDGLDEEDWDGWKYMTQELGEKIQLVGDDLFVTNTERLGKGIKMGCGNSILIKLNQIGSVSETLEAIKMAHKAGYTAVTSHRSGETEDTTIADLAVALNTGQIKTGAPSRSERVAKYNQLLRIEEELGESAVYPGVGTF

SEQ ID NO: 25 - DNA, 3525 bp, Anaerostipes rhamnosivorans
atggcaagaaaaatgaaaaccatggatggtaacacagctgcagcacacgtgtcttacgcttttactgagacagctgctatctacccaattacaccatcctcaccaatggcagaatcaacagaccagtgggcaacacagggaaagaaaaatattttcggacatcctgtaaaattagttgaaatgcagtcagagggcggagcagcaggaactgttcacggatctcttgcagcaggcgcattgacaactacatacactgcatctcagggattattactgatgattcctaatatgtacaagatcgcaggtgaattaatgccttgtgtatttgatgtatctgcacgtgcaatcgcatctcatgcactgaatatctttggtgatcactctgacgtttacgcttgtcgccagacaggttttgcaatgctttgcagctccagcgttcaggaagttatggacttaggcgctgttgctcatttatctacaatcaagggaagcgttccagttctccatttcttcgatggattcagaacatctcatgagatccagaagattgaaatgtgggacaacgaagatcttgcagatatggtagattacgatgctcttgaaaagttcagagcaaatgctttgaatccagaacatccggttctccgcggatctgctcagaatccagatatcttcttccaggcaagagaagcatgcaacaaatactacgatgcattcccggcaatcgtcgaagattacatgaaccaggttaatgcaaagatcggtacagattataaattattcaactactacggagctgctgatgctgaatctgttatcattgcaatgggatctgtatgtgagactatcgatgagaccatcgattacttaacagcaaaaggcgaaaaagtcggtgttgttaaagtaagactttacagaccattcgtagctgaaaaattactcgaagttatcccagagactgttaaaacaatcaacgttttagacagaacaaaagaaccgggatctcttggagagccattatacttagacgttgtttctgcattaagaggaacaaaattcgagcaggtaacaatcaacggaggacgttacggattaggatccaaagatacaactcctgctgatatcatctctgtattcaataacaaagataagaaagaattcacagttgcgatcaacgatgatgtgacaaacttatctcttgaaagaggagaagctgtttcttctacaccagaaagcacagtaagctgtaagttctggggtcttggagcagatggtaccgttggtgctaacaagaactccatcaagatcatcggtgaccacactgacaaatatgctcaggcatactttgattatgattccaagaaatccggtggtgttacaatttcccacttacgtttcggcgatgacccgatcaaagcaacttacctgatcaacaaagctgattttgttgcatgtcactgtgctccgtacatgacaaaatacaacatggttcaggacttaaaagatggcggaacattcttgttaaactgtagctggccggtagaagaaatcggagatcatcttccaggacaggtaaaagcttacatggcaaaacacaacatcaagttctatgtgatcgatggtatcaagatcggtaaagagattggacttggcggacgtatcaatactgtacttcagtctgcattcttcaaattatctggaattattccggaagcagacgcgatccagtatatgaaagacgctgctactgcttcttacagcaagaagggtgatcacatcgttaagatgaatcatgacgcgatcgaagctggtgctaagagtgttgtagaagttccggttcctgctgaatgggctgactgcggagaagaaagcttattacatccagtatccggagatagaaaagatcttgttgattatgttaacaacattcagaacgctgtcaatggacagattggtaacagccttaaggtaagcgacttagttccttacgctgacggtcatatgcctcagggatctgctgcatacgaaaaacgtggagttgctgtagacgttcctacatggaatccggacaactgtatccagtgtaacttctgtgcatatgtatgtcctcatgcagttatccgtcctgttgccatgacagaagcagaacttgctgcagcaccggaagctacaaaatctcttccgatgacgggaatgccagattacaaatttgtaatgacaactactgttcttgactgtacaggatgtggttcttgtgcaaatgtctgcccaggtaagaaaggcgagaaagctcttacaatggcaccactcgacagcatgagagatcagcaggaagtttacaactatggacagtctttacctataaaagaagatatctttgacaagtttaaaccagaatctgttaagggaagccagttcaagaaacctctccttgagttctcaggagcttgtgcaggatgtggagagactccatatgcgaaactgatcactcagttattcggagacagaatgtacattgccaatgcgacaggatgttcttctatctggggtggttcttcacctgcaacaccttacacagtaaacagcgaaggacagggaccggcttggtctaactcactgttcgaagacaatgctgagtttggtttaggtatgttcacaggacagaatactttaagagagcagacaaaagaaaaggttgcagcattagcagaatctacagataatgcagatgtaaaagcagcagcggctgaatacttagatacttacacagacggtaagaaaaacggagcagctactaagaaattagttgcagcactggaagcttgcggatgtgaagaaggaaaagaaatccttaagatgaaagattacttatctaagaaatctcagtggatcttcggtggtgacggatgggcttacgacatcggctttggcggtgttgaccatgtacttgcaagtggtgaagatgttaatatctttgtattcgatacagaggtttactctaacaccggcggacaggcatccaaatctacaccggcaggtgcgatcgctcagttcgcagcagcaggtaaggctgtgaagaaaaaagaccttgcagcaatcgcaatgagctacggatatgtatatgttgctcagatctctatgggtgctgattacaatcagacagtcaaagcgatcgcagaggcagaagcttatccaggaccatcactgatcatcggttatgctccatgtatcaaccacggtatcaagggtggaatgagcattgctcagtctgaagaaaagaaagcagttacatcaggatactggaacttattccgtttcaaccctgaattagctgaagaaggaaagaatccattctcattagattctaaagagcctacaacagagtacaatgacttcatcatgggtgaggttcgttacaacgctcttgctcgccagaatcctgagagagctaaggagctgtttgagcaggcagaaaagaatgccaaagaaaaatatgctcgtttgaaagaattagcagataaataa

SEQ ID NO: 26 - protein, 1174 aa, Anaerostipes rhamnosivorans
MARKMKTMDGNTAAAHVSYAFTETAAIYPITPSSPMAESTDQWATQGKKNIFGHPVKLVEMQSEGGAAGTVHGSLAAGALTTTYTASQGLLLMIPNMYKIAGELMPCVFDVSARAIASHALNIFGDHSDVYACRQTGFAMLCSSSVQEVMDLGAVAHLSTIKGSVPVLHFFDGFRTSHEIQKIEMWDNEDLADMVDYDALEKFRANALNPEHPVLRGSAQNPDIFFQAREACNKYYDAFPAIVEDYMNQVNAKIGTDYKLFNYYGAADAESVIIAMGSVCETIDETIDYLTAKGEKVGVVKVRLYRPFVAEKLLEVIPETVKTINVLDRTKEPGSLGEPLYLDVVSALRGTKFEQVTINGGRYGLGSKDTTPADIISVFNNKDKKEFTVAINDDVTNLSLERGEAVSSTPESTVSCKFWGLGADGTVGANKNSIKIIGDHTDKYAQAYFDYDSKKSGGVTISHLRFGDDPIKATYLINKADFVACHCAPYMTKYNMVQDLKDGGTFLLNCSWPVEEIGDHLPGQVKAYMAKHNIKFYVIDGIKIGKEIGLGGRINTVLQSAFFKLSGIIPEADAIQYMKDAATASYSKKGDHIVKMNHDAIEAGAKSVVEVPVPAEWADCGEESLLHPVSGDRKDLVDYVNNIQNAVNGQIGNSLKVSDLVPYADGHMPQGSAAYEKRGVAVDVPTWNPDNCIQCNFCAYVCPHAVIRPVAMTEAELAAAPEATKSLPMTGMPDYKFVMTTTVLDCTGCGSCANVCPGKKGEKALTMAPLDSMRDQQEVYNYGQSLPIKEDIFDKFKPESVKGSQFKKPLLEFSGACAGCGETPYAKLITQLFGDRMYIANATGCSSIWGGSSPATPYTVNSEGQGPAWSNSLFEDNAEFGLGMFTGQNTLREQTKEKVAALAESTDNADVKAAAAEYLDTYTDGKKNGAATKKLVAALEACGCEEGKEILKMKDYLSKKSQWIFGGDGWAYDIGFGGVDHVLASGEDVNIFVFDTEVYSNTGGQASKSTPAGAIAQFAAAGKAVKKKDLAAIAMSYGYVYVAQISMGADYNQTVKAIAEAEAYPGPSLIIGYAPCINHGIKGGMSIAQSEEKKAVTSGYWNLFRFNPELAEEGKNPFSLDSKEPTTEYNDFIMGEVRYNALARQNPERAKELFEQAEKNAKEKYARLKELADK

SEQ ID NO: 27 - DNA, 1560 bp, Anaerostipes rhamnosivorans
atgggaaaagttaaaattatcacagcagatcaggctgctgccctggtggaggatcatacaacgatcacaaccagcggatttgttgccagcggaatgccggaagctctcacaaaagctttggaaaaaagattcaaagaaaccggatcccccaaaaacctgactttgttttatgctgccgcccagggaaaccgtgacggaagcggcgcggatcattttgcccatgaaggtatgacaaaacgtgtcataggcggacattggaacatggttcctgcactgggacagatggttctggacaataaaatcgaaggctataatctgcctcagggaacactggcacagttataccgtgccatcgcaggtcacaaacccggcgttatcagtcatgtaggattaaatacatttgctgacccacgcattgagggcggtaaattaaatgacattacaaccgaagatatcgtcgatgtagttgaaatcctgggagaagagaaattattgtacaaatcgttcccgatcaacatcggatttatccgcggtacatatgcagatgaacacggcaacgtaactctgtctcacgaatgtgccaccacagaggtcacaaccatggctcaggccgtaaagaacagcggcggaaaggtcgttgtacaggtagaaaaggtcgtttcagacggcaccttagatccaaaattagtcaagatccctggaatttatgtggatgccgttgtggaagtagaagacatgaaggatcacgaacaatgcgtgggctgtgattatgatggttccatgaccggagacttccgggttccgttaagcagtcttgagtacccgcctctttccgccaagaagatcattggacgcagagcagccatggaactgacagaaaacacggttgtaaatctaggcattggtattccagagtacatctccatggtagccaacgaagaaggcattggagattacatgactctgactgtagaggccggaccaatcggcggtgtacctcagggaggagctaaattcggaggcgctgtcaatgctgaatgtatcctagatcagccgtaccagtttgatttctatgacggcggcggagttgactatgcattcttaggactcgcacaggcagataaagacggtaatatcaacgtaagcaagttcggtccaaggattgcgggctgcggcggtttcgtcaatatcacccagaacgcaaaacgctgctacttctgcggaacttttactgcaggaggtctgaagacatccgtaaaagacgggaaacttatcattgaccaggaaggaaaatcaaacaaattcctggataccgtagaacagatcaccttcagcggtgaatacgcaaataaagtcggccagccggttctctatatcacagaacgtgctgtattcgaactcagaaaggacggcgtctatctgacagaggttgcccctggtatcgatatccagacacagatcatcgatcatatgggatttgctccaaagatggaaggaacgccaaagttcatggatgaaagaatcttcaaagatgagctgatgggcctgaagcacgattaa

SEQ ID NO: 28 - protein, 519 aa, Anaerostipes rhamnosivorans
MGKVKIITADQAAALVEDHTTITTSGFVASGMPEALTKALEKRFKETGSPKNLTLFYAAAQGNRDGSGADHFAHEGMTKRVIGGHWNMVPALGQMVLDNKIEGYNLPQGTLAQLYRAIAGHKPGVISHVGLNTFADPRIEGGKLNDITTEDIVDVVEILGEEKLLYKSFPINIGFIRGTYADEHGNVTLSHECATTEVTTMAQAVKNSGGKVVVQVEKVVSDGTLDPKLVKIPGIYVDAVVEVEDMKDHEQCVGCDYDGSMTGDFRVPLSSLEYPPLSAKKIIGRRAAMELTENTVVNLGIGIPEYISMVANEEGIGDYMTLTVEAGPIGGVPQGGAKFGGAVNAECILDQPYQFDFYDGGGVDYAFLGLAQADKDGNINVSKFGPRIAGCGGFVNITQNAKRCYFCGTFTAGGLKTSVKDGKLIIDQEGKSNKFLDTVEQITFSGEYANKVGQPVLYITERAVFELRKDGVYLTEVAPGIDIQTQIIDHMGFAPKMEGTPKFMDERIFKDELMGLKHD

SEQ ID NO: 29 - DNA, 1143 bp, Anaerostipes rhamnosivorans
atgaattttgaattaacagagcagcaggcagccattcaggaaacagcaaggaactttgctcagacagaactgcagccaggtgtgctggagagagatgcaaacagtgagtttccggtagatctttataagaagatgggggatatgggcctgatcggtcttccatatccgaaagaagtcggaggacagggagccgattatctttcttatgcactggcagtggaagagatctcaaaagtagatgcctctgtgggtatttcctattctgtttccacctctctgtatggcggaagcattatgaactctgatgcttccgttgagaaaaagaatgaatttctagcaccggtactgtccggacagcatttcggatcctttggactcacagagcccaatgccggatcagacgcaggcggatgtgtcactgtggctgaaaaagatggggacgaatacatattaaatggtatgaagtgttttaacaccaacggacctttggccgattatacagcggtatatgccctgacagaacctgagaagaaagcaaagggtctttcctgctttgtagtaaaaaaaggaactcctggattttctgtaggcaaggtggaggataagatgggaatccgctctgcgcaggtctctgagatgatcctggaaaatgtcagagtacctgcagagaatatggtcgttccatctggggacggatttaagctggccatgaagactttggatggaggacgcatcggcgttgccgcgcaggggcttggtatcgcggagggcgcatttgagatcgcaaaagaatacttaaagagcagagaacagttcggcaaaccattatataaaaaccagtatcttgcatttaagatggcggaacttgaaatggagatcgacgctgcaagatacatgctttacaaagcggctacagataaacacgagggaagatcttactcaatcccggcagcaaaggcaaaatacctttgtacagaggccgctatgcatgtgacaacagaagcagtacagatgttaggtggaaatgggtatatgaagggttaccatgtagaacgtatgatgagagatgcgaagatcactcagatctatgagggaaccaatgaaatccagaagctcgttgtaagcggagctatcttcagatag

SEQ ID NO: 30 - protein, 380 aa, Anaerostipes rhamnosivorans
MNFELTEQQAAIQETARNFAQTELQPGVLERDANSEFPVDLYKKMGDMGLIGLPYPKEVGGQGADYLSYALAVEEISKVDASVGISYSVSTSLYGGSIMNSDASVEKKNEFLAPVLSGQHFGSFGLTEPNAGSDAGGCVTVAEKDGDEYILNGMKCFNTNGPLADYTAVYALTEPEKKAKGLSCFVVKKGTPGFSVGKVEDKMGIRSAQVSEMILENVRVPAENMVVPSGDGFKLAMKTLDGGRIGVAAQGLGIAEGAFEIAKEYLKSREQFGKPLYKNQYLAFKMAELEMEIDAARYMLYKAATDKHEGRSYSIPAAKAKYLCTEAAMHVTTEAVQMLGGNGYMKGYHVERMMRDAKITQIYEGTNEIQKLVVSGAIFR

SEQ ID NO: 31 - DNA, 777 bp, Anaerostipes rhamnosivorans
atgaacaatttattaatggaagtagaaaacgaagtcgcagttgtaacaatcaacagaccaaaatcattaaacgctttaaacagtgaaactttagctgagttagaccagtgctttacagagatttcaggacgcaaggacatcagagtggtgatcctcacaggatcaggcgaaaaatcctttgtagctggtgctgatatctctgaaatggtcaatgcaactcctgcagaaggaagacagatgggtcttttggcaaaagaagcatttcttaagttagagacaatgcctcaggtaaccatcgctgccgtcaacggttacgctctgggaggcggatgcgagatctccatggcatgtgatatccgtgtagctgcggaaaacgcaaaattcgcacagccagaaacaggacttggaattcttccgggattcggcggaacacagcgtctctcccgtttagtaggaaaaggacgcgcaaaggaattgatctttacatgtgaccagatcgatgccgaagaagcttacagaattggtcttgcaaacaaagtagtgcctcaggcggagctgatcgattactgtaagaagatggctgcaaagatcatgtccaaaggaagctacgcaatttcccttgcaaaagaagcgatcaacacaggaatggacacagacttaagcagcggtcttacattagaagctgacttattcggacttgcattctccactgctgacaaaaaagagggtatgacagcattccttgaaaaacgtaaagctgatttaaaagatttctag

SEQ ID NO: 32 - protein, 258 aa, Anaerostipes rhamnosivorans
MNNLLMEVENEVAVVTINRPKSLNALNSETLAELDQCFTEISGRKDIRVVILTGSGEKSFVAGADISEMVNATPAEGRQMGLLAKEAFLKLETMPQVTIAAVNGYALGGGCEISMACDIRVAAENAKFAQPETGLGILPGFGGTQRLSRLVGKGRAKELIFTCDQIDAEEAYRIGLANKVVPQAELIDYCKKMAAKIMSKGSYAISLAKEAINTGMDTDLSSGLTLEADLFGLAFSTADKKEGMTAFLEKRKADLKDF

SEQ ID NO: 33 - DNA, 1143 bp, Anaerostipes rhamnosivorans
atggttgaatttaaattgacaaaagaacagctgcttgcacagcagatgttcaaagatttcgcagagacagaaatcaagccaattgctagggacatggatgaaacagaagaattcagcatggaactgcttgagaaaatgaaaaaattcggtcttatgggaattccatattccaaagaatacggcggagaaggggcagacgttttaacttatacactctgtatggaagagatgtctaagttagatgcttccacaggaatcacaatttccgtacatacatcattgtgctgttcctgcatcaatgattttgcttcagaagagcagaaacaaaaatttttacgtccgttggttgacggttctaaagtgggatgctttggtcttacagagccgggagcaggtaccgatgccagcggtgtgaagacaaatgccgtattggacggcgatgaatatatcttaaacggacagaaggtgttcactacaaactcaggctttgccgatacatttattatttttgcagttacagacaaaactgcaggtacaagaggtatgtctgcatttatcgtggacaggaatacaccgggattagaagtcagcaagaatatccctcgtatgggtatccgtgctgcgtcaaactgtgaagttgctttagttgatgtgagagtaccgaaggaaaacctgatcgcgggcgagggaaaaggatataagatcgctatgtcagcgctggcaggcggacggatcggaatcggcgctcagtccgtaggaatcgcacagggtgctttaaacgaggccattgcatatgtaaaggaaagaaaacagttcggaaaaccgatttctaaattccagaatacacagttcctgatcgcagaactccaagtgaagatcgacgctgcgcgccttatggtatggagagcggcaaaatcaaaagacgatcatgaaaactatgctcctctggcagcagcgtgcaaactgtttgcatctgacgtggcagtggaagtgacaagagcagccgtacagcttcttggaggatatggatactccagagaatatccggtagagagaatgtaccgtgatgctaagatcacagagatttacgaaggtacatctgaagctatgaagatgatcgtcggcggcggacttttaagataa

SEQ ID NO: 34 - protein, 380 aa, Anaerostipes rhamnosivorans
MVEFKLTKEQLLAQQMFKDFAETEIKPIARDMDETEEFSMELLEKMKKFGLMGIPYSKEYGGEGADVLTYTLCMEEMSKLDASTGITISVHTSLCCSCINDFASEEQKQKFLRPLVDGSKVGCFGLTEPGAGTDASGVKTNAVLDGDEYILNGQKVFTTNSGFADTFIIFAVTDKTAGTRGMSAFIVDRNTPGLEVSKNIPRMGIRAASNCEVALVDVRVPKENLIAGEGKGYKIAMSALAGGRIGIGAQSVGIAQGALNEAIAYVKERKQFGKPISKFQNTQFLIAELQVKIDAARLMVWRAAKSKDDHENYAPLAAACKLFASDVAVEVTRAAVQLLGGYGYSREYPVERMYRDAKITEIYEGTSEAMKMIVGGGLLR

SEQ ID NO: 35 - DNA, 798 bp, Anaerostipes rhamnosivorans
atgaaaatcgttgtttgtgcaaaacaagtaccagatacaactgaagtaaaattagatccaaagaccaacactctgatcagggatggtgttccgagtatcatcaacccagatgacaaagccggaatcgaagctgccctgcagttaaaggaaaaatgcccgggaagcactgtgactgttgtttccatgggaccgcctcaggcagatgtggcattgagagaagcacttgctatgggatgtgacgatgcagttctggtttctgacagagcattcggaggagcagatacatgggcaacatcttccactatcgcggcagcgatcaaaaaattggattacgatgtcatcatcacaggacgccaggcgattgacggagataccgctcaggtaggccctcagatcgcagaacatcttggaattcctcaggtgagctatgtcgaagccctgaatgtggaaggggaagacactttgatcgtccagaggcagtttgaagaccgccatcagatcattgaagtaaagactccttgcctgatcactgctttggctgaattggcaacacctagatatatgacagtccacggtgcatttgacgcatacagagaaaaagaagtcagcgtctggggacttgcagacttagaagacactgtagaccaggcaaatatcgggttaaaaggatctcctacaagagtaaagaaatcctttacaaaacaggccaaaggtgcaggaactattttaaaagatttatcagcagacgaagctgttgcatccattgttgataaacttcaggaaaagcacatcatttaa

SEQ ID NO: 36 - protein, 265 aa, Anaerostipes rhamnosivorans
MKIVVCAKQVPDTTEVKLDPKTNTLIRDGVPSIINPDDKAGIEAALQLKEKCPGSTVTVVSMGPPQADVALREALAMGCDDAVLVSDRAFGGADTWATSSTIAAAIKKLDYDVIITGRQAIDGDTAQVGPQIAEHLGIPQVSYVEALNVEGEDTLIVQRQFEDRHQIIEVKTPCLITALAELATPRYMTVHGAFDAYREKEVSVWGLADLEDTVDQANIGLKGSPTRVKKSFTKQAKGAGTILKDLSADEAVASIVDKLQEKHII

SEQ ID NO: 37 - DNA, 996 bp, Anaerostipes rhamnosivorans
atgagcagagacgtatatgtattcgctgagcagagagacggcgaacttcagaaagtcggaatcgaattaattggtaaagcaagagaattagcagatgatcttggacagcaggtcgttgccgtgctgcttggggcaggcgtaaaagaaaaagcccaggaattagttgcctgcggtgcagataaagttgttgtcgtagatgacattatgctggcagattatgtgacagagccatacacaaaggcactgacagctgtgattaacgcaaaagatcctgagatcgtgctttatggtgcatcttccatcggacgtgaccttgccccaagagtatctgcaaggatccatactggattgacagcggactgtactgcacttgccattgatgaagagacaaagctcctcatgatgacccgtccggcatttggcggaaatatcatggcgaccatcgtgtgtgaagacttccgtcctcagatggcaacagtccgtccaggagtaatgaaagccttggaaaacgatgcatccagaagcggtgaagtggaagagttcaaagtagaattctctgatgcagacatgaatgtaaaagtccgtgaaactgtaaagacagcacataaatctgtagacattacagaggctaagatccttgtttccggtgggcgcggaatcggaagcgctgacaagttcaaggtgttagaggaactggcggatgtcatggaaggtgaagtgtcttcttccagagcatgtgtagattccggatggatcagtgctgaccgccaggtaggacagacaggaaagactgtgcgtccggaattatatctggcctgcggcatctcaggagcgatccagcatgcagccggtatggagaactcagagtttatcgtagccatcaaccgtgatgaggatgcgccgatctttgatattgcagacctcggcatcgttggagacttaaataccatcgtaccgaaacttacagaagctgtgaaggcggtaaaaggaaaataa

SEQ ID NO: 38 - protein, 331 aa, Anaerostipes rhamnosivorans
MSRDVYVFAEQRDGELQKVGIELIGKARELADDLGQQVVAVLLGAGVKEKAQELVACGADKVVVVDDIMLADYVTEPYTKALTAVINAKDPEIVLYGASSIGRDLAPRVSARIHTGLTADCTALAIDEETKLLMMTRPAFGGNIMATIVCEDFRPQMATVRPGVMKALENDASRSGEVEEFKVEFSDADMNVKVRETVKTAHKSVDITEAKILVSGGRGIGSADKFKVLEELADVMEGEVSSSRACVDSGWISADRQVGQTGKTVRPELYLACGISGAIQHAAGMENSEFIVAINRDEDAPIFDIADLGIVGDLNTIVPKLTEAVKAVKGK

SEQ ID NO: 39 - DNA, 834 bp, Anaerostipes rhamnosivorans
atgaaaatcggatttaacgaagcaactgcactggaatgtaaaggacagtctctgatggctgacttagaagcgtgtgaaaaatacggatttgattacatcgagatcagatttgactgtgtaaaggattatttgaaagaacacacactggaagaactggcggactggtttaaaaaccatcatttaaaaccatgggcttataataccttgatcttttttaaccagagagacgaagccggagaaaaagagattgacgaagagactgattttatcattgaagtgtgcaaggccatcggcatgaagatgctgatcactgttccgtcctttgatgtaaaggataagagcatcacagagatcaaagaggaagcagtagaacgtctccgttatttatccgataaggtgggagaagatattaagatatcactggaattctgcggggcgccgaactgctccatcaaccagtttgggacagcttacgatgtagtgaaagcaacagaccgtgacaatgtaggggtaacagtggataccttccatttccatgagatgtgttctaagttagaagatttagaagcagcggacggaaagaagatctttgcttaccatctgaatgactgtgaggatctgcctctgggatcatgcggtgatgataaacgcctgtggccgggagaaggtgtggtagatcatgcaggaatcgcagctgccttaaagaagatcggattcgacggggtatgtaccattgaagaattccgtccagaatattatgaaatgtcccatgacgagaatgtgaaaaagtctgctgaagtgacaagggatttcgtagcaaaatactttgcataa

SEQ ID NO: 40 - protein, 277 aa, Anaerostipes rhamnosivorans
MKIGFNEATALECKGQSLMADLEACEKYGFDYIEIRFDCVKDYLKEHTLEELADWFKNHHLKPWAYNTLIFFNQRDEAGEKEIDEETDFIIEVCKAIGMKMLITVPSFDVKDKSITEIKEEAVERLRYLSDKVGEDIKISLEFCGAPNCSINQFGTAYDVVKATDRDNVGVTVDTFHFHEMCSKLEDLEAADGKKIFAYHLNDCEDLPLGSCGDDKRLWPGEGVVDHAGIAAALKKIGFDGVCTIEEFRPEYYEMSHDENVKKSAEVTRDFVAKYFA

SEQ ID NO: 41 - DNA, 945 bp, Anaerostipes rhamnosivorans
atgacatataaaatgagaaaaatagtgatcataggggccggacatgtgggaaccatgtgcggcctgagtcttatgtaccgcggagaagtggacgaattggtattcatagacatcgataaggaaaaggcatacagccaggcgctggaccttagtgatgccgtctccttcctgccccggcagatcacggtgcgcaccggggattatgaagatgtggaggacgctgacatcatcgttatggccgctggtgtttccaggctgcccggtcagacaaggctggatatgctggacgattccatccgcatcatgaaagaagtcaccagccatttaaaagataagaagattcagggcatcctcatcagtatctccaatcctgccgacattgtggcggattatctgagaaaacaactgggccttccgagaaaccgctgcttcagcaccggtacttccctggactcctgccgtctcagaaggattctttcccaaagaatgcacttagaccggaattccattcaggcctttgtcatgggagaacacggagattcccaaatgatcccgttttctgcggtacatatagccgggaaaccattgcttcagtggatgcaagatgaaccggaacggtacggaagtctcgatctttcacagattgaaacggagaccgccaacgccggccatgaagtgatcgaaggaaagggcagcacagagtttggtatcggcatcgccctgtccgagatcgtccagtccattttccacgattcaaaaaaggtccttcccgcttcgcctctgctggaaggagaatatggacagcatggcgttcacgccggtgtcccctgcgtgattggaaaagccggaattgaaaagatcattgagatccctctcacagaccgggagcaagagaaatttgaccattcctgcgatgtgatcagaacgtttattcaagatctctga

SEQ ID NO: 42 - protein, 314 aa, Anaerostipes rhamnosivorans
MTYKMRKIVIIGAGHVGTMCGLSLMYRGEVDELVFIDIDKEKAYSQALDLSDAVSFLPRQITVRTGDYEDVEDADIIVMAAGVSRLPGQTRLDMLDDSIRIMKEVTSHLKDKKIQGILISISNPADIVADYLRKQLGLPRNRCFSTGTSLDSCRLRRILSQRMHLDRNSIQAFVMGEHGDSQMIPFSAVHIAGKPLLQWMQDEPERYGSLDLSQIETETANAGHEVIEGKGSTEFGIGIALSEIVQSIFHDSKKVLPASPLLEGEYGQHGVHAGVPCVIGKAGIEKIIEIPLTDREQEKFDHSCDVIRTFIQDL

SEQ ID NO: 43 - DNA, 1176 bp, Anaerostipes rhamnosivorans
atgggaagatttacattacctagagatttgtactttggaaaaggatcacttgagacactgaagacgctggacggaaaaaaagctgttttagtcatcggcggcggatctatgaaacgttttggatttgtagaccaggttgttgattatttaaaagaagcaggcatagagacaaaattaattgagggagtagagccggacccttctgttgagaccgtatttaaaggtgccgagattatgagagagtttgaaccggactggatcgtggcaatgggcggaggatctccgatcgatgctgcaaaagctatgtggatcttctatgaacatccggaaaccacatttgacgatgtaaaagacccattcacagttccaaaactcagaaacaaagcaagattccttgcaatcccttcaacttcaggaacagctacggaagtgacggcatttgctgttatcacggactacagttcaggcattaaatatccgctggcagactttgagatcacaccggatgttgccattgtagaccctgcactggcagagacgatgcctccgaaactgacctcttatacaggaatggatgcactgacccatgcgattgaagcatatgttgccggactgcattcaccattttcagatcctttggcaatcgatgccatcgagatggtatttgacaacttaaaagcttcctatgagggagacatgaccgcacgtgaaaatatgcactatgcacaatgtcttgcaggaatgtcattctctaatgcactgttaggaattgtacattctttagcgcataagacaggagcagtttttgatacaggacatatcacacacggatgtgcgaacgctatctatcttccgtatgtgatccagtttaatgcaaaggtatgcgaagaccgctatgcagccattgcaagaaagatcggcctccctggaaattccgataaagcactgacactttctttgattgaagcaatccgggatatgaacaagaagctgaacattgtttccaacctgaaagatttcggaatcaatgaagaagaattcaaccagaagaagaggatgatcgcggagcgtgctgtggcagacgcatgtacaggatctaatcctagagaaacctctgtagatgacatggaaaaagtgttagagtgcatctactacggaaaagacgtaatattctaa

SEQ ID NO: 44 - protein, 391 aa, Anaerostipes rhamnosivorans
MGRFTLPRDLYFGKGSLETLKTLDGKKAVLVIGGGSMKRFGFVDQVVDYLKEAGIETKLIEGVEPDPSVETVFKGAEIMREFEPDWIVAMGGGSPIDAAKAMWIFYEHPETTFDDVKDPFTVPKLRNKARFLAIPSTSGTATEVTAFAVITDYSSGIKYPLADFEITPDVAIVDPALAETMPPKLTSYTGMDALTHAIEAYVAGLHSPFSDPLAIDAIEMVFDNLKASYEGDMTARENMHYAQCLAGMSFSNALLGIVHSLAHKTGAVFDTGHITHGCANAIYLPYVIQFNAKVCEDRYAAIARKIGLPGNSDKALTLSLIEAIRDMNKKLNIVSNLKDFGINEEEFNQKKRMIAERAVADACTGSNPRETSVDDMEKVLECIYYGKDVIF

SEQ ID NO: 45 - DNA, 1184 bp, Anaerostipes rhamnosivorans
cgaaagtccgtgaatttttgtgccttaccgcacatcctcagggctgcagaaaaaatgttgaagatcagattacatatgtaaaaaaacacagcggtgttgagaatgggccgaaaaaggtcctggtcatcggctcatccacgggatatggcctggcatcaaggatcaccgctgcattcgggtgcggagcggataccttgggtgtcatgtttgagagaccggcggccggtaaacgtaccgccactgccggatggtataatacagctgcatttgaacaggcggcggccagagaaggcttatatgcgaagacggtcaacgcagatgcattttccagggaagtaaaagaccaggtcatccaactgatcaagaaggatttcggtaaggttgacgctgtcatttacagtctggctgcaccgagaagaacgacagaggacggcacggtttatcagtctgtcttaaagacaacaggggaagattttacaaataagaacctgaaccttaaagataataccatcagtgagaaaaccatcgaggctgccacagaggaagaagtgaaagctaccgtgaaggtcatgggcggggaggactggaaggcatggatccaggcgcttaccgatgcggatgccattgaaaagggcgcagtcaccttcgcatattcttatatcggacctaagcttacatatcctatctattttgagggaaccatcggtgccgcgaagaagcatcttcataagacggcgggtgagatcaatgaggaattctcaaaggatggaatcaaagcatacatctccgtcaataaggggctggtgacacaggccagttctgccattccgatcgtaccgctttatatggctgtgctgtaccgcgttatgaaagaaaagggacttcatgaaggctgtatcgaacagatcgtaaggctgttttctgaaaaggctgtctttggacaggccaagacagatgaacagggtctgatccgtctggatgactgggaactgcgcccggaagtccaggaagaagtggaacgccgctgggagaaggtctgcaccgaaaatgtccaggagtattctgatattgacggatactgggatgatttctaccaaatgttcggcttccgtatggatggagtggactacgaggaggatcttgacccttctgtggatatcccaagcatttccggccaagaatag

SEQ ID NO: 46 - protein, 398 aa, Anaerostipes rhamnosivorans
MKISPKVREFLCLTAHPQGCRKNVEDQITYVKKHSGVENGPKKVLVIGSSTGYGLASRITAAFGCGADTLGVMFERPAAGKRTATAGWYNTAAFEQAAAREGLYAKTVNADAFSREVKDQVIQLIKKDFGKVDAVIYSLAAPRRTTEDGTVYQSVLKTTGEDFTNKNLNLKDNTISEKTIEAATEEEVKATVKVMGGEDWKAWIQALTDADAIEKGAVTFAYSYIGPKLTYPIYFEGTIGAAKKHLHKTAGEINEEFSKDGIKAYISVNKGLVTQASSAIPIVPLYMAVLYRVMKEKGLHEGCIEQIVRLFSEKAVFGQAKTDEQGLIRLDDWELRPEVQEEVERRWEKVCTENVQEYSDIDGYWDDFYQMFGFRMDGVDYEEDLDPSVDIPSISGQE

SEQ ID NO: 47 - DNA, 1434 bp, Anaerostipes rhamnosivorans
atgctgacaaaaaaaacaaagattgtttgcacgatggggcctgcgacagatgatgatgcggtgctcaaacagctgatgctggaaggaatggatatcgcgaggctgaatttctctcacggtgaccatgaggaacagcttggaaggatcaagagaattaaaagattccgtgaagaactgaatattccgatcgcgatcttactggatacaaaaggacctgagatccgtacaggacttctggagacagacggagacgtggaattaaaagaaggaaatgaatttgttctgaccacaagggacatcaaaggaaatgattctatcgtagcaattacatacgaagagcttccgcaggatgtcacagaaggaaatacaatcctgatcgatgacggtctgatcgaactgagagtcaaagagatcaaagacggaacagatattgtatgtgaaattgtaaacggcggacttttaggaagccgtaagggagtgaacgtaccgaacgtgcgcgtaaatcttccttccatcacagaaaaggataagagagacattgagttcggacttgaaaacggcattgattttatcgcggcttctttcatccgtaacgcagatgcagtcaatgagatcaaggacatcgtaaaagcccacaacatggaagtcggcgttatctccaaaattgaaaacgtggagggtgtggaaaatattgaatccatcatccaggcttctgacggaatcatggtagcccgtggagatttaggtgtggaaattcccgcagaggaagttccgtttatgcagaaagcaatcattaagaaatgcaacaatgcatttaagccagtcatcacagctacacagatgttagactctatgatcagaaacccacgtccgacaagagcggaagtaacagacgttgccaatgctatttatgacggaaccgatgcgattatgctttctggagaaaccgcgaaggggaaataccctgtggaggccgtaaggatgatgaaccagatcgccatctccacagaactgcatctggatacgaagatcaaagattttcgttccatctatgtaagccgtggaatctcagcagcggttgcttattctgctgtacagactgctcataatattaacgcaaaatgtatcctggcgtccagtatgtccggatttacggcaaggatcgtatctaagttcaaaccggatgcacagatcattggcttgtctccggatgaagcggttgtgcgtaagatgcagctttactggggagtgcgtccgtttaagtcacataaggcttcctctaccagagaacttttggatgaagctacggatatcgtactgaatgcagatcttgcacaggagaacgacatcctggtactgaccggaggaggtccggcagaccaccgtggaaaaggcgtgacaaacatgctgaaagtagtaaaaatcggcgaataa

SEQ ID NO: 48 - protein, 477 aa, Anaerostipes rhamnosivorans
MLTKKTKIVCTMGPATDDDAVLKQLMLEGMDIARLNFSHGDHEEQLGRIKRIKRFREELNIPIAILLDTKGPEIRTGLLETDGDVELKEGNEFVLTTRDIKGNDSIVAITYEELPQDVTEGNTILIDDGLIELRVKEIKDGTDIVCEIVNGGLLGSRKGVNVPNVRVNLPSITEKDKRDIEFGLENGIDFIAASFIRNADAVNEIKDIVKAHNMEVGVISKIENVEGVENIESIIQASDGIMVARGDLGVEIPAEEVPFMQKAIIKKCNNAFKPVITATQMLDSMIRNPRPTRAEVTDVANAIYDGTDAIMLSGETAKGKYPVEAVRMMNQIAISTELHLDTKIKDFRSIYVSRGISAAVAYSAVQTAHNINAKCILASSMSGFTARIVSKFKPDAQIIGLSPDEAVVRKMQLYWGVRPFKSHKASSTRELLDEATDIVLNADLAQENDILVLTGGGPADHRGKGVTNMLKVVKIGE